SEQUENCE LISTING (1090 sequences)

SEQ ID NO: 1 - protein, 9 aa, Human betaherpesvirus 5
NLVPMVATV

SEQ ID NO: 2 - protein, 9 aa, Human gammaherpesvirus 4
CLGGLLTMV

SEQ ID NO: 3 - protein, 9 aa, Influenza A
GILGFVFTL

SEQ ID NO: 4 - protein, 10 aa, Epstein-Barr virus
AVFDRKSDAK

SEQ ID NO: 5 - protein, 9 aa, Human betaherpesvirus 5
QYDPVAALF

SEQ ID NO: 6 - protein, 11 aa, Human gammaherpesvirus 4
TPSVSSSISSL

SEQ ID NO: 7 - protein, 9 aa, Severe acute respiratory syndrome coronavirus 2
LPFNDGVYF

SEQ ID NO: 8 - protein, 9 aa, Human gammaherpesvirus 4
FLGERVTLT

SEQ ID NO: 9 - protein, 9 aa, Severe acute respiratory syndrome coronavirus 2
LLALHRSYL

SEQ ID NO: 10 - protein, 9 aa, Human adenovirus 5
STDVASLNY

SEQ ID NO: 11 - protein, 8 aa, Human papillomavirus
TLGIVCPI

SEQ ID NO: 12 - protein, 8 aa, Human gammaherpesvirus 4
RYSIFFDY

SEQ ID NO: 13 - protein, 8 aa, Human gammaherpesvirus 4
CPLSKILL

SEQ ID NO: 14 - protein, 8 aa, Human herpesvirus 3
SLGYITTV

SEQ ID NO: 15 - protein, 8 aa, Human herpesvirus 4
RAKFKQLL

SEQ ID NO: 16 - protein, 8 aa, Human herpesvirus 6
TNKIKEQL

SEQ ID NO: 17 - protein, 8 aa, Human herpesvirus 6
QTRQKFHL

SEQ ID NO: 18 - protein, 8 aa, Human herpesvirus 6
EMRLRMIL

SEQ ID NO: 19 - protein, 8 aa, Human herpesvirus 6
MAREKNDL

SEQ ID NO: 20 - protein, 8 aa, Human papillomavirus
CCKCDSTL

SEQ ID NO: 21 - protein, 8 aa, Human papillomavirus
HDIILECV

SEQ ID NO: 22 - protein, 8 aa, Severe acute respiratory syndrome coronavirus 2
LPQGFSAL

SEQ ID NO: 23 - protein, 8 aa, Severe acute respiratory syndrome coronavirus 2
NLTTRTQL

SEQ ID NO: 24 - protein, 8 aa, Severe acute respiratory syndrome coronavirus 2
AEHVNNSY

SEQ ID NO: 25 - protein, 8 aa, Severe acute respiratory syndrome coronavirus 2
AEVQIDRL

SEQ ID NO: 26 - protein, 8 aa, Severe acute respiratory syndrome coronavirus 2
EPLVDLPI

SEQ ID NO: 27 - protein, 8 aa, Severe acute respiratory syndrome coronavirus 2
FPREGVFV

SEQ ID NO: 28 - protein, 8 aa, Severe acute respiratory syndrome coronavirus 2
NITRFQTL

SEQ ID NO: 29 - protein, 8 aa, Severe acute respiratory syndrome coronavirus 2
QPYRVVVL

SEQ ID NO: 30 - protein, 8 aa, Severe acute respiratory syndrome coronavirus 2
RLQSLQTY

SEQ ID NO: 31 - protein, 8 aa, Severe acute respiratory syndrome coronavirus 2
SPRRARSV

SEQ ID NO: 32 - protein, 8 aa, Severe acute respiratory syndrome coronavirus 2
VYYPDKVF

SEQ ID NO: 33 - protein, 8 aa, Severe acute respiratory syndrome coronavirus 2
YAWNRKRI

SEQ ID NO: 34 - protein, 8 aa, Severe acute respiratory syndrome coronavirus 2
FLPFFSNV

SEQ ID NO: 35 - protein, 8 aa, Severe acute respiratory syndrome coronavirus 2
APHGVVFL

SEQ ID NO: 36 - protein, 8 aa, Severe acute respiratory syndrome coronavirus 2
GVYHKNNK

SEQ ID NO: 37 - protein, 9 aa, Human adenovirus 11
TYFNLGNKF

SEQ ID NO: 38 - protein, 9 aa, Human adenovirus 5
TDLGQNLLY

SEQ ID NO: 39 - protein, 9 aa, Human adenovirus 5
TLLYVLFEV

SEQ ID NO: 40 - protein, 9 aa, Human adenovirus 5
VLAWTRAFV

SEQ ID NO: 41 - protein, 9 aa, Human alphaherpesvirus 1
YVAGFLALY

SEQ ID NO: 42 - protein, 9 aa, Human alphaherpesvirus 1
ALMGAVTSL

SEQ ID NO: 43 - protein, 9 aa, Human alphaherpesvirus 1
MMLRDRWSL

SEQ ID NO: 44 - protein, 9 aa, Human alphaherpesvirus 1
AVLCLYLLY

SEQ ID NO: 45 - protein, 9 aa, Human alphaherpesvirus 1
FLWEDQTLL

SEQ ID NO: 46 - protein, 9 aa, Human alphaherpesvirus 1
FIPQYLSAV

SEQ ID NO: 47 - protein, 9 aa, Human alphaherpesvirus 1
FLIAYQPLL

SEQ ID NO: 48 - protein, 9 aa, Human alphaherpesvirus 1
SVYPYDEFV

SEQ ID NO: 49 - protein, 9 aa, Human alphaherpesvirus 1
VYMSPFYGY

SEQ ID NO: 50 - protein, 9 aa, Human alphaherpesvirus 1
FLGDDPSPA

SEQ ID NO: 51 - protein, 9 aa, Human alphaherpesvirus 1
RLTGYPAGI

SEQ ID NO: 52 - protein, 9 aa, Human alphaherpesvirus 1
APASVYQPA

SEQ ID NO: 53 - protein, 9 aa, Human alphaherpesvirus 1
CPRRPAVAF

SEQ ID NO: 54 - protein, 9 aa, Human alphaherpesvirus 1
VVRGPTVSL

SEQ ID NO: 55 - protein, 9 aa, Human alphaherpesvirus 1
LIDGIFLRY

SEQ ID NO: 56 - protein, 9 aa, Human alphaherpesvirus 1
SQLAHLVYV

SEQ ID NO: 57 - protein, 9 aa, Human alphaherpesvirus 1
FLGAGALAV

SEQ ID NO: 58 - protein, 9 aa, Human alphaherpesvirus 1
FLGGHVAVA

SEQ ID NO: 59 - protein, 9 aa, Human alphaherpesvirus 1
TLRGLFFSV

SEQ ID NO: 60 - protein, 9 aa, Human alphaherpesvirus 1
KYFYCNSLF

SEQ ID NO: 61 - protein, 9 aa, Human alphaherpesvirus 1
EYQRLYATF

SEQ ID NO: 62 - protein, 9 aa, Human alphaherpesvirus 1
APRIGGRRA

SEQ ID NO: 63 - protein, 9 aa, Human alphaherpesvirus 1
APRTWCRLL

SEQ ID NO: 64 - protein, 9 aa, Human alphaherpesvirus 1
ALMLRLLRI

SEQ ID NO: 65 - protein, 9 aa, Human alphaherpesvirus 1
RILGVLVHL

SEQ ID NO: 66 - protein, 9 aa, Human alphaherpesvirus 1
YMESVFQMY

SEQ ID NO: 67 - protein, 9 aa, Human alphaherpesvirus 1
ILIEGIFFA

SEQ ID NO: 68 - protein, 9 aa, Human alphaherpesvirus 1
YMANQILRY

SEQ ID NO: 69 - protein, 9 aa, Human alphaherpesvirus 1
VPRPDDPVL

SEQ ID NO: 70 - protein, 9 aa, Human alphaherpesvirus 1
VYTPSPYVF

SEQ ID NO: 71 - protein, 9 aa, Human alphaherpesvirus 1
AYLPRPVEF

SEQ ID NO: 72 - protein, 9 aa, Human alphaherpesvirus 1
AILTQYWKY

SEQ ID NO: 73 - protein, 9 aa, Human alphaherpesvirus 1
ATDSLNNEY

SEQ ID NO: 74 - protein, 9 aa, Human alphaherpesvirus 1
AYVSVLYRW

SEQ ID NO: 75 - protein, 9 aa, Human alphaherpesvirus 1
LASDPHYEY

SEQ ID NO: 76 - protein, 9 aa, Human alphaherpesvirus 1
LLAYVSVLY

SEQ ID NO: 77 - protein, 9 aa, Human alphaherpesvirus 1
RLNELLAYV

SEQ ID NO: 78 - protein, 9 aa, Human alphaherpesvirus 1
SIVHHHAQY

SEQ ID NO: 79 - protein, 9 aa, Human alphaherpesvirus 1
ALATVTLKY

SEQ ID NO: 80 - protein, 9 aa, Human alphaherpesvirus 1
ALLDRDCRV

SEQ ID NO: 81 - protein, 9 aa, Human alphaherpesvirus 1
FLADAVVRL

SEQ ID NO: 82 - protein, 9 aa, Human alphaherpesvirus 1
FTAPEVGTY

SEQ ID NO: 83 - protein, 9 aa, Human alphaherpesvirus 1
RLLGFADTV

SEQ ID NO: 84 - protein, 9 aa, Human alphaherpesvirus 1
RSSLGSLLY

SEQ ID NO: 85 - protein, 9 aa, Human alphaherpesvirus 1
ALHTALATV

SEQ ID NO: 86 - protein, 9 aa, Human alphaherpesvirus 1
TLLELVVSV

SEQ ID NO: 87 - protein, 9 aa, Human alphaherpesvirus 1
VPGWSRRTL

SEQ ID NO: 88 - protein, 9 aa, Human alphaherpesvirus 1
ALLAKMLFY

SEQ ID NO: 89 - protein, 9 aa, Human alphaherpesvirus 1
RMLGDVMAV

SEQ ID NO: 90 - protein, 9 aa, Human alphaherpesvirus 1
TMLEDHEFV

SEQ ID NO: 91 - protein, 9 aa, Human alphaherpesvirus 1
ALLGLTLGV

SEQ ID NO: 92 - protein, 9 aa, Human alphaherpesvirus 1
FVLATGDFV

SEQ ID NO: 93 - protein, 9 aa, Human alphaherpesvirus 1
GIFEDRAPV

SEQ ID NO: 94 - protein, 9 aa, Human alphaherpesvirus 1
LLTTPKFTV

SEQ ID NO: 95 - protein, 9 aa, Human alphaherpesvirus 1
NLLTTPKFT

SEQ ID NO: 96 - protein, 9 aa, Human alphaherpesvirus 1
TMYYKDVTV

SEQ ID NO: 97 - protein, 9 aa, Human alphaherpesvirus 1
YLANGGFLI

SEQ ID NO: 98 - protein, 9 aa, Human alphaherpesvirus 1
ALSALLTKL

SEQ ID NO: 99 - protein, 9 aa, Human alphaherpesvirus 1
FLTCTDRSV

SEQ ID NO: 100 - protein, 9 aa, Human alphaherpesvirus 2
DRLDNRLQL

SEQ ID NO: 101 - protein, 9 aa, Human alphaherpesvirus 2
KSRRPLTTF

SEQ ID NO: 102 - protein, 9 aa, Human alphaherpesvirus 2
RRAQMAPKR

SEQ ID NO: 103 - protein, 9 aa, Human alphaherpesvirus 2
GPHETITAL

SEQ ID NO: 104 - protein, 9 aa, Human alphaherpesvirus 2
PAWSRRTLL

SEQ ID NO: 105 - protein, 9 aa, Human alphaherpesvirus 2
ASDSLNNEY

SEQ ID NO: 106 - protein, 9 aa, Human alphaherpesvirus 2
FLVDAIVRV

SEQ ID NO: 107 - protein, 9 aa, Human alphaherpesvirus 2
GLADTVVAC

SEQ ID NO: 108 - protein, 9 aa, Human alphaherpesvirus 2
RPRGEVRFL

SEQ ID NO: 109 - protein, 9 aa, Human alphaherpesvirus 3
SAPLPSNRV

SEQ ID NO: 110 - protein, 9 aa, Human alphaherpesvirus 3
SLPRSRTPI

SEQ ID NO: 111 - protein, 9 aa, Human alphaherpesvirus 3
ALWALPHAA

SEQ ID NO: 112 - protein, 9 aa, Human alphaherpesvirus 3
ILIEGIFFV

SEQ ID NO: 113 - protein, 9 aa, Human betaherpesvirus 5
VTEHDTLLY

SEQ ID NO: 114 - protein, 9 aa, Human betaherpesvirus 5
ELNRKMIYM

SEQ ID NO: 115 - protein, 9 aa, Human betaherpesvirus 5
IPSINVHHY

SEQ ID NO: 116 - protein, 9 aa, Human betaherpesvirus 5
TRATKMQVI

SEQ ID NO: 117 - protein, 9 aa, Human betaherpesvirus 5
QIKVRVKMV

SEQ ID NO: 118 - protein, 9 aa, Human betaherpesvirus 5
DELRRKMMY

SEQ ID NO: 119 - protein, 9 aa, Human betaherpesvirus 5
EEAIVAYTL

SEQ ID NO: 120 - protein, 9 aa, Human betaherpesvirus 5
ELKRKMMYM

SEQ ID NO: 121 - protein, 9 aa, Human betaherpesvirus 5
QIKVRVDMV

SEQ ID NO: 122 - protein, 9 aa, Human betaherpesvirus 5
VLEETSVML

SEQ ID NO: 123 - protein, 9 aa, Human betaherpesvirus 5
ARAKKDELK

SEQ ID NO: 124 - protein, 9 aa, Human betaherpesvirus 5
ARAKKDELR

SEQ ID NO: 125 - protein, 9 aa, Human betaherpesvirus 5
DELKRKMIY

SEQ ID NO: 126 - protein, 9 aa, Human betaherpesvirus 5
FMDILTTCV

SEQ ID NO: 127 - protein, 9 aa, Human coronavirus 229E
KEWAYCVEM

SEQ ID NO: 128 - protein, 9 aa, Human coronavirus 229E
LITGRLAAL

SEQ ID NO: 129 - protein, 9 aa, Human coronavirus 229E
LLLNCLWSV

SEQ ID NO: 130 - protein, 9 aa, Human coronavirus OC43
TMLDIQPED

SEQ ID NO: 131 - protein, 9 aa, Human coronavirus OC43
LPRWYFYYL

SEQ ID NO: 132 - protein, 9 aa, Human coronavirus OC43
KLWHYCSTL

SEQ ID NO: 133 - protein, 9 aa, Human gammaherpesvirus 4
LLIEGIFFI

SEQ ID NO: 134 - protein, 9 aa, Human gammaherpesvirus 4
AFLGERVTL

SEQ ID NO: 135 - protein, 9 aa, Human gammaherpesvirus 4
KLGPGEEQV

SEQ ID NO: 136 - protein, 9 aa, Human gammaherpesvirus 4
RFIAQLLLL

SEQ ID NO: 137 - protein, 9 aa, Human gammaherpesvirus 4
TLTSYWRRV

SEQ ID NO: 138 - protein, 9 aa, Human gammaherpesvirus 4
VEDLFGANL

SEQ ID NO: 139 - protein, 9 aa, Human gammaherpesvirus 4
WQWEHIPPA

SEQ ID NO: 140 - protein, 9 aa, Human gammaherpesvirus 4
LPCVLWPVL

SEQ ID NO: 141 - protein, 9 aa, Human gammaherpesvirus 4
RAKFKQLLQ

SEQ ID NO: 142 - protein, 9 aa, Human gammaherpesvirus 4
SENDRLRLL

SEQ ID NO: 143 - protein, 9 aa, Human gammaherpesvirus 4
TLDTKPLSV

SEQ ID NO: 144 - protein, 9 aa, Human gammaherpesvirus 4
FMVFLQTHI

SEQ ID NO: 145 - protein, 9 aa, Human gammaherpesvirus 4
HPVGEADYF

SEQ ID NO: 146 - protein, 9 aa, Human gammaherpesvirus 4
VLKDAIKDL

SEQ ID NO: 147 - protein, 9 aa, Human gammaherpesvirus 4
AYSSWMYSY

SEQ ID NO: 148 - protein, 9 aa, Human gammaherpesvirus 4
QAKWRLQTL

SEQ ID NO: 149 - protein, 9 aa, Human gammaherpesvirus 4
RLRAEAQVK

SEQ ID NO: 150 - protein, 9 aa, Human gammaherpesvirus 4
SVRDRLARL

SEQ ID NO: 151 - protein, 9 aa, Human gammaherpesvirus 4
IVTDFSVIK

SEQ ID NO: 152 - protein, 9 aa, Human gammaherpesvirus 4
VSFIEFVGW

SEQ ID NO: 153 - protein, 9 aa, Human gammaherpesvirus 4
EGGVGWRHW

SEQ ID NO: 154 - protein, 9 aa, Human gammaherpesvirus 4
QPRAPIRPI

SEQ ID NO: 155 - protein, 9 aa, Human gammaherpesvirus 4
RRIYDLIEL

SEQ ID NO: 156 - protein, 9 aa, Human gammaherpesvirus 4
RPPIFIRLL

SEQ ID NO: 157 - protein, 9 aa, Human gammaherpesvirus 4
YPLHEQHGM

SEQ ID NO: 158 - protein, 9 aa, Human gammaherpesvirus 4
LMIIPLINV

SEQ ID NO: 159 - protein, 9 aa, Human gammaherpesvirus 4
SLVIVTTFV

SEQ ID NO: 160 - protein, 9 aa, Human gammaherpesvirus 4
TLFIGSHVV

SEQ ID NO: 161 - protein, 9 aa, Human gammaherpesvirus 4
YLQQNWWTL

SEQ ID NO: 162 - protein, 9 aa, Human gammaherpesvirus 4
ALLVLYSFA

SEQ ID NO: 163 - protein, 9 aa, Human gammaherpesvirus 4
GLGTLGAAL

SEQ ID NO: 164 - protein, 9 aa, Human gammaherpesvirus 4
LLSAWILTA

SEQ ID NO: 165 - protein, 9 aa, Human gammaherpesvirus 4
LLWTLVVLL

SEQ ID NO: 166 - protein, 9 aa, Human gammaherpesvirus 4
MGSLEMVPM

SEQ ID NO: 167 - protein, 9 aa, Human gammaherpesvirus 4
YLLEMLWRL

SEQ ID NO: 168 - protein, 9 aa, Human gammaherpesvirus 4
VLQWASLAV

SEQ ID NO: 169 - protein, 9 aa, Human gammaherpesvirus 4
FLYALALLL

SEQ ID NO: 170 - protein, 9 aa, Human gammaherpesvirus 4
IEDPPFNSL

SEQ ID NO: 171 - protein, 9 aa, Human gammaherpesvirus 4
RRRWRRLTV

SEQ ID NO: 172 - protein, 9 aa, Human gammaherpesvirus 4
TYGPVFMCL

SEQ ID NO: 173 - protein, 9 aa, Human gammaherpesvirus 4
FLRGRAYGL

SEQ ID NO: 174 - protein, 9 aa, Human gammaherpesvirus 8
LVLILYLCV

SEQ ID NO: 175 - protein, 9 aa, Human gammaherpesvirus 8
LLNGWRWRL

SEQ ID NO: 176 - protein, 9 aa, Human gammaherpesvirus 8
IGLITVLFL

SEQ ID NO: 177 - protein, 9 aa, Human herpesvirus 3
DAAPAIQHI

SEQ ID NO: 178 - protein, 9 aa, Human herpesvirus 3
KQYLGVYIW

SEQ ID NO: 179 - protein, 9 aa, Human herpesvirus 3
TYATFLVTW

SEQ ID NO: 180 - protein, 9 aa, Human herpesvirus 3
KENIAAYKF

SEQ ID NO: 181 - protein, 9 aa, Human herpesvirus 3
KTNNWHAGW

SEQ ID NO: 182 - protein, 9 aa, Human herpesvirus 3
APKTATSSW

SEQ ID NO: 183 - protein, 9 aa, Human herpesvirus 3
QTTGRITNR

SEQ ID NO: 184 - protein, 9 aa, Human herpesvirus 3
VEDINRVFL

SEQ ID NO: 185 - protein, 9 aa, Human herpesvirus 4
CYDHAQTHL

SEQ ID NO: 186 - protein, 9 aa, Human herpesvirus 4
TLDYKPLSV

SEQ ID NO: 187 - protein, 9 aa, Human herpesvirus 4
YRSGIIAVV

SEQ ID NO: 188 - protein, 9 aa, Human herpesvirus 4
RPPIFIRRL

SEQ ID NO: 189 - protein, 9 aa, Human herpesvirus 4
FLDKGTYTL

SEQ ID NO: 190 - protein, 9 aa, Human herpesvirus 4
ARYAYYLQF

SEQ ID NO: 191 - protein, 9 aa, Human herpesvirus 4
RRRKGWIPL

SEQ ID NO: 192 - protein, 9 aa, Human herpesvirus 4
LQHYREVAA

SEQ ID NO: 193 - protein, 9 aa, Human herpesvirus 4
AENAGNDAC

SEQ ID NO: 194 - protein, 9 aa, Human herpesvirus 4
RVRAYTYSK

SEQ ID NO: 195 - protein, 9 aa, Human herpesvirus 4
YVLDHLIVV

SEQ ID NO: 196 - protein, 9 aa, Human herpesvirus 4
DEVEFLGHY

SEQ ID NO: 197 - protein, 9 aa, Human herpesvirus 4
GLCTLVAML

SEQ ID NO: 198 - protein, 9 aa, Human herpesvirus 4
KDTWLDARM

SEQ ID NO: 199 - protein, 9 aa, Human herpesvirus 4
VLFGLLCLL

SEQ ID NO: 200 - protein, 9 aa, Human herpesvirus 5
ELRRKMMYM

SEQ ID NO: 201 - protein, 9 aa, Human herpesvirus 5
ELKRKMIYM

SEQ ID NO: 202 - protein, 9 aa, Human herpesvirus 6
EFKSKFSTL

SEQ ID NO: 203 - protein, 9 aa, Human herpesvirus 6
EGRDRILTV

SEQ ID NO: 204 - protein, 9 aa, Human herpesvirus 6
FEKERFLFL

SEQ ID NO: 205 - protein, 9 aa, Human herpesvirus 6
EGRERILTV

SEQ ID NO: 206 - protein, 9 aa, Human herpesvirus 6
NVKHKKNPL

SEQ ID NO: 207 - protein, 9 aa, Human herpesvirus 6
VVKGKVLSI

SEQ ID NO: 208 - protein, 9 aa, Human herpesvirus 6
EARRRLAEM

SEQ ID NO: 209 - protein, 9 aa, Human herpesvirus 6
RSKPRHMCV

SEQ ID NO: 210 - protein, 9 aa, Human herpesvirus 6
DFKSKYLTL

SEQ ID NO: 211 - protein, 9 aa, Human herpesvirus 6
SPRSRLQQL

SEQ ID NO: 212 - protein, 9 aa, Human metapneumovirus
VLATAVREL

SEQ ID NO: 213 - protein, 9 aa, Human metapneumovirus
FQANTPPAV

SEQ ID NO: 214 - protein, 9 aa, Human metapneumovirus
IPYTAAVQV

SEQ ID NO: 215 - protein, 9 aa, Human metapneumovirus
KLAKLIIDL

SEQ ID NO: 216 - protein, 9 aa, Human metapneumovirus
RLPREKLKK

SEQ ID NO: 217 - protein, 9 aa, Human metapneumovirus
SPKAGLLSL

SEQ ID NO: 218 - protein, 9 aa, Human metapneumovirus
SLQQEITLL

SEQ ID NO: 219 - protein, 9 aa, Human orthopneumovirus
ISDYFHNTY

SEQ ID NO: 220 - protein, 9 aa, human papillomavirus 11
KVLIRCYLC

SEQ ID NO: 221 - protein, 9 aa, human papillomavirus 11
SIDQLCKTF

SEQ ID NO: 222 - protein, 9 aa, human papillomavirus 44
HNNGICWGN

SEQ ID NO: 223 - protein, 9 aa, Human papillomavirus 52
CYEQLGDSS

SEQ ID NO: 224 - protein, 9 aa, Human papillomavirus 52
ITIRCIICQ

SEQ ID NO: 225 - protein, 9 aa, Human papillomavirus 52
KTLEERVKK

SEQ ID NO: 226 - protein, 9 aa, Human papillomavirus 52
MRGDKATIK

SEQ ID NO: 227 - protein, 9 aa, Human papillomavirus 52
PYGVCIMCL

SEQ ID NO: 228 - protein, 9 aa, Human papillomavirus 52
QLGDSSDEE

SEQ ID NO: 229 - protein, 9 aa, Human papillomavirus 52
RLQCVQCKK

SEQ ID NO: 230 - protein, 9 aa, Human papillomavirus 52
VYKFLFTDL

SEQ ID NO: 231 - protein, 9 aa, Human papillomavirus 52
YYYAGSSRL

SEQ ID NO: 232 - protein, 9 aa, Human papillomavirus 52
LQFIFQLCK

SEQ ID NO: 233 - protein, 9 aa, Human papillomavirus 52
MTLCAEVKK

SEQ ID NO: 234 - protein, 9 aa, Human papillomavirus 52
QYRVFRIKL

SEQ ID NO: 235 - protein, 9 aa, Human papillomavirus 52
RIKLPDPNK

SEQ ID NO: 236 - protein, 9 aa, Human papillomavirus 58
ILIRCIICQ

SEQ ID NO: 237 - protein, 9 aa, Human papillomavirus 58
KCLNEILIR

SEQ ID NO: 238 - protein, 9 aa, Human papillomavirus 58
KVCLRLLSK

SEQ ID NO: 239 - protein, 9 aa, Human papillomavirus 58
ATEVRTLQQ

SEQ ID NO: 240 - protein, 9 aa, Human papillomavirus 58
CTIVCPSCA

SEQ ID NO: 241 - protein, 9 aa, Human papillomavirus 58
LCINSTATE

SEQ ID NO: 242 - protein, 9 aa, Human papillomavirus 58
AVPDDLYIK

SEQ ID NO: 243 - protein, 9 aa, Human papillomavirus 58
KYTFWEVNL

SEQ ID NO: 244 - protein, 9 aa, Human papillomavirus 58
RVRLPDPNK

SEQ ID NO: 245 - protein, 9 aa, Human papillomavirus 58
TSESQLFNK

SEQ ID NO: 246 - protein, 9 aa, Human papillomavirus 58
YTFWEVNLK

SEQ ID NO: 247 - protein, 9 aa, Human papillomavirus type 16
YLTAPTGCI

SEQ ID NO: 248 - protein, 9 aa, Human papillomavirus type 16
DSAPILTAF

SEQ ID NO: 249 - protein, 9 aa, Human papillomavirus type 16
KSAIVTLTY

SEQ ID NO: 250 - protein, 9 aa, Human papillomavirus type 16
LAVSKNKAL

SEQ ID NO: 251 - protein, 9 aa, Human papillomavirus type 16
LQDVSLEVY

SEQ ID NO: 252 - protein, 9 aa, Human papillomavirus type 16
NPCHTTKLL

SEQ ID NO: 253 - protein, 9 aa, Human papillomavirus type 16
NTTPIVHLK

SEQ ID NO: 254 - protein, 9 aa, Human papillomavirus type 16
QVILCPTSV

SEQ ID NO: 255 - protein, 9 aa, Human papillomavirus type 16
RLECAIYYK

SEQ ID NO: 256 - protein, 9 aa, Human papillomavirus type 16
SPEIIRQHL

SEQ ID NO: 257 - protein, 9 aa, Human papillomavirus type 16
TLYTAVSST

SEQ ID NO: 258 - protein, 9 aa, Human papillomavirus type 16
VVEGQVDYY

SEQ ID NO: 259 - protein, 9 aa, Human papillomavirus type 16
YRFKKHCTL

SEQ ID NO: 260 - protein, 9 aa, Human papillomavirus type 16
ALQAIELQL

SEQ ID NO: 261 - protein, 9 aa, Human papillomavirus type 16
TLQDVSLEV

SEQ ID NO: 262 - protein, 9 aa, Human papillomavirus type 16
YIIFVYIPL

SEQ ID NO: 263 - protein, 9 aa, Human papillomavirus type 16
FAFRDLCIV

SEQ ID NO: 264 - protein, 9 aa, Human papillomavirus type 16
IILECVYCK

SEQ ID NO: 265 - protein, 9 aa, Human papillomavirus type 16
TTLEQQYNK

SEQ ID NO: 266 - protein, 9 aa, Human papillomavirus type 16
VCDKCLKFY

SEQ ID NO: 267 - protein, 9 aa, Human papillomavirus type 16
CPEEKQRHL

SEQ ID NO: 268 - protein, 9 aa, Human papillomavirus type 16
YGTTLEQQY

SEQ ID NO: 269 - protein, 9 aa, Human papillomavirus type 16
EYRHYCYSL

SEQ ID NO: 270 - protein, 9 aa, Human papillomavirus type 16
VYDFAFQDL

SEQ ID NO: 271 - protein, 9 aa, Human papillomavirus type 16
GTLGIVCPI

SEQ ID NO: 272 - protein, 9 aa, Human papillomavirus type 16
IVCPICSQK

SEQ ID NO: 273 - protein, 9 aa, Human papillomavirus type 16
QAEPDRAHY

SEQ ID NO: 274 - protein, 9 aa, Human papillomavirus type 16
RAHYNIVTF

SEQ ID NO: 275 - protein, 9 aa, Human papillomavirus type 16
TLHEYMLDL

SEQ ID NO: 276 - protein, 9 aa, Human papillomavirus type 16
LQPETTDLY

SEQ ID NO: 277 - protein, 9 aa, Human papillomavirus type 16
TPTLHEYML

SEQ ID NO: 278 - protein, 9 aa, Human papillomavirus type 16
LLMGTLGIV

SEQ ID NO: 279 - protein, 9 aa, Human papillomavirus type 16
TLGIVCPIC

SEQ ID NO: 280 - protein, 9 aa, Human papillomavirus type 16
IHSMNSTIL

SEQ ID NO: 281 - protein, 9 aa, Human papillomavirus type 16
ISEYRHYCY

SEQ ID NO: 282 - protein, 9 aa, Human papillomavirus type 16
KFYSKISEY

SEQ ID NO: 283 - protein, 9 aa, Human papillomavirus type 16
KLPQLCTEL

SEQ ID NO: 284 - protein, 9 aa, Human papillomavirus type 16
VYDFAFRDL

SEQ ID NO: 285 - protein, 9 aa, Human papillomavirus type 16
TIHDIILEC

SEQ ID NO: 286 - protein, 9 aa, Human papillomavirus type 16
YMLDLQPET

SEQ ID NO: 287 - protein, 9 aa, Human papillomavirus type 18
IHSMNSSIL

SEQ ID NO: 288 - protein, 9 aa, Human papillomavirus type 18
NVFPIFLQM

SEQ ID NO: 289 - protein, 9 aa, Human papillomavirus type 18
KLPDLCTEL

SEQ ID NO: 290 - protein, 9 aa, Human papillomavirus type 18
NLLIRCLRC

SEQ ID NO: 291 - protein, 9 aa, Human papillomavirus type 18
FQQLFLNTL

SEQ ID NO: 292 - protein, 9 aa, Human papillomavirus type 18
LFLNTLSFV

SEQ ID NO: 293 - protein, 9 aa, Human papillomavirus type 6
LLLGTLNIV

SEQ ID NO: 294 - protein, 9 aa, Human papillomavirus type 6b
TIDQLCKTF

SEQ ID NO: 295 - protein, 9 aa, Human parvovirus B19
EADVQQWLT

SEQ ID NO: 296 - protein, 9 aa, Human parvovirus B19
FYTPLADQF

SEQ ID NO: 297 - protein, 9 aa, Human parvovirus B19
GLCPHCINV

SEQ ID NO: 298 - protein, 9 aa, Human parvovirus B19
GLFNNVLYH

SEQ ID NO: 299 - protein, 9 aa, Human parvovirus B19
LHTDFEQVM

SEQ ID NO: 300 - protein, 9 aa, Human parvovirus B19
LLHTDFEQV

SEQ ID NO: 301 - protein, 9 aa, Human parvovirus B19
SESSFFNLI

SEQ ID NO: 302 - protein, 9 aa, Human parvovirus B19
SSHSGSFQI

SEQ ID NO: 303 - protein, 9 aa, Human parvovirus B19
TEADVQQWL

SEQ ID NO: 304 - protein, 9 aa, Human parvovirus B19
VQQWLTWCN

SEQ ID NO: 305 - protein, 9 aa, Human parvovirus B19
TAKSRVHPL

SEQ ID NO: 306 - protein, 9 aa, Human polyomavirus 1
LLLIWFRPV

SEQ ID NO: 307 - protein, 9 aa, Human polyomavirus 1
AVDTVLAKK

SEQ ID NO: 308 - protein, 9 aa, Human polyomavirus 1
EPLVWIDCY

SEQ ID NO: 309 - protein, 9 aa, Human polyomavirus 1
AITEVECFL

SEQ ID NO: 310 - protein, 9 aa, Human polyomavirus 1
LLMWEAVTV

SEQ ID NO: 311 - protein, 9 aa, Human respiratory syncytial virus A2
RARRELPRF

SEQ ID NO: 312 - protein, 9 aa, Human respiratory syncytial virus A2
YLEKESIYY

SEQ ID NO: 313 - protein, 9 aa, Human respiratory syncytial virus A2
NPKASLLSL

SEQ ID NO: 314 - protein, 9 aa, Human respiratory syncytial virus A2
QVMLRWGVL

SEQ ID NO: 315 - protein, 9 aa, Influenza A virus
GILGFVFTL

SEQ ID NO: 316 - protein, 9 aa, Influenza A virus
SRYWAIRTR

SEQ ID NO: 317 - protein, 9 aa, Influenza A virus
NMLSTVLGV

SEQ ID NO: 318 - protein, 9 aa, Influenza A virus
LPFERATVM

SEQ ID NO: 319 - protein, 9 aa, Influenza A virus
LPFERATIM

SEQ ID NO: 320 - protein, 9 aa, Influenza A virus
LPFDKSTVM

SEQ ID NO: 321 - protein, 9 aa, Influenza A virus
LPFEKSTIM

SEQ ID NO: 322 - protein, 9 aa, Influenza A virus
LPFDKPTIM

SEQ ID NO: 323 - protein, 9 aa, Influenza A virus
LPFEKSTVM

SEQ ID NO: 324 - protein, 9 aa, Influenza A virus
CVNGSCFTV

SEQ ID NO: 325 - protein, 9 aa, Influenza A virus
LPFDRTTIM

SEQ ID NO: 326 - protein, 9 aa, Influenza A virus
KTGGPIYRR

SEQ ID NO: 327 - protein, 9 aa, Influenza A virus
LPFDRPTIM

SEQ ID NO: 328 - protein, 9 aa, Influenza B virus
GLDNHTILL

SEQ ID NO: 329 - protein, 9 aa, JC polyomavirus
ILMWEAVTL

SEQ ID NO: 330 - protein, 9 aa, JC polyomavirus
SITEVECFL

SEQ ID NO: 331 - protein, 9 aa, Severe acute respiratory syndrome coronavirus 2
NLNESLIDL

SEQ ID NO: 332 - protein, 9 aa, Severe acute respiratory syndrome coronavirus 2
RLDKVEAEV

SEQ ID NO: 333 - protein, 9 aa, Severe acute respiratory syndrome coronavirus 2
VYDPLQPEL

SEQ ID NO: 334 - protein, 9 aa, Severe acute respiratory syndrome coronavirus 2
TLKSFTVEK

SEQ ID NO: 335 - protein, 9 aa, Severe acute respiratory syndrome coronavirus 2
HADQLTPTW

SEQ ID NO: 336 - protein, 9 aa, Severe acute respiratory syndrome coronavirus 2
NATNVVIKV

SEQ ID NO: 337 - protein, 9 aa, Severe acute respiratory syndrome coronavirus 2
VGYLQPRTF

SEQ ID NO: 338 - protein, 9 aa, Severe acute respiratory syndrome coronavirus 2
YFQPRTFLL

SEQ ID NO: 339 - protein, 9 aa, Severe acute respiratory syndrome coronavirus 2
FQFCNDPFL

SEQ ID NO: 340 - protein, 9 aa, Severe acute respiratory syndrome coronavirus 2
ASVYAWNRK

SEQ ID NO: 341 - protein, 9 aa, Severe acute respiratory syndrome coronavirus 2
GYLQPRTFL

SEQ ID NO: 342 - protein, 9 aa, Severe acute respiratory syndrome coronavirus 2
YLQLRTFLL

SEQ ID NO: 343 - protein, 9 aa, Severe acute respiratory syndrome coronavirus 2
YLQPRIFLL

SEQ ID NO: 344 - protein, 9 aa, Severe acute respiratory syndrome coronavirus 2
AEVQIDRLI

SEQ ID NO: 345 - protein, 9 aa, Severe acute respiratory syndrome coronavirus 2
ALNTLVKQL

SEQ ID NO: 346 - protein, 9 aa, Severe acute respiratory syndrome coronavirus 2
CVADYSVLY

SEQ ID NO: 347 - protein, 9 aa, Severe acute respiratory syndrome coronavirus 2
GSFCTQLNR

SEQ ID NO: 348 - protein, 9 aa, Severe acute respiratory syndrome coronavirus 2
GVVFLHVTY

SEQ ID NO: 349 - protein, 9 aa, Severe acute respiratory syndrome coronavirus 2
LLFNKVTLA

SEQ ID NO: 350 - protein, 9 aa, Severe acute respiratory syndrome coronavirus 2
LLQYGSFCT

SEQ ID NO: 351 - protein, 9 aa, Severe acute respiratory syndrome coronavirus 2
RLQSLQTYV

SEQ ID NO: 352 - protein, 9 aa, Severe acute respiratory syndrome coronavirus 2
RVDFCGKGY

SEQ ID NO: 353 - protein, 9 aa, Severe acute respiratory syndrome coronavirus 2
SVLNDILSR

SEQ ID NO: 354 - protein, 9 aa, Severe acute respiratory syndrome coronavirus 2
VLNDILSRL

SEQ ID NO: 355 - protein, 9 aa, Severe acute respiratory syndrome coronavirus 2
FVFKNIDGY

SEQ ID NO: 356 - protein, 9 aa, Severe acute respiratory syndrome coronavirus 2
GTHWFVTQR

SEQ ID NO: 357 - protein, 9 aa, Severe acute respiratory syndrome coronavirus 2
GTITSGWTF

SEQ ID NO: 358 - protein, 9 aa, Severe acute respiratory syndrome coronavirus 2
KEIDRLNEV

SEQ ID NO: 359 - protein, 9 aa, Severe acute respiratory syndrome coronavirus 2
LEPLVDLPI

SEQ ID NO: 360 - protein, 9 aa, Severe acute respiratory syndrome coronavirus 2
LPPAYTNSF

SEQ ID NO: 361 - protein, 9 aa, Severe acute respiratory syndrome coronavirus 2
NGVEGFNCY

SEQ ID NO: 362 - protein, 9 aa, Severe acute respiratory syndrome coronavirus 2
NQKLIANQF

SEQ ID NO: 363 - protein, 9 aa, Severe acute respiratory syndrome coronavirus 2
RISNCVADY

SEQ ID NO: 364 - protein, 9 aa, Severe acute respiratory syndrome coronavirus 2
RLFRKSNLK

SEQ ID NO: 365 - protein, 9 aa, Severe acute respiratory syndrome coronavirus 2
TLDSKTQSL

SEQ ID NO: 366 - protein, 9 aa, Severe acute respiratory syndrome coronavirus 2
TPINLVRDL

SEQ ID NO: 367 - protein, 9 aa, Severe acute respiratory syndrome coronavirus 2
YFPLQSYGF

SEQ ID NO: 368 - protein, 9 aa, Severe acute respiratory syndrome coronavirus 2
FQPTNGVGY

SEQ ID NO: 369 - protein, 9 aa, Severe acute respiratory syndrome coronavirus 2
RFDNPVLPF

SEQ ID NO: 370 - protein, 9 aa, Severe acute respiratory syndrome coronavirus 2
TSNQVAVLY

SEQ ID NO: 371 - protein, 9 aa, Severe acute respiratory syndrome coronavirus 2
GLTVLPPLL

SEQ ID NO: 372 - protein, 9 aa, Severe acute respiratory syndrome coronavirus 2
SIIAYTMSL

SEQ ID NO: 373 - protein, 9 aa, Severe acute respiratory syndrome coronavirus 2
YLQPRTFLL

SEQ ID NO: 374 - protein, 9 aa, Severe acute respiratory syndrome coronavirus 2
EPVLKGVKL

SEQ ID NO: 375 - protein, 9 aa, Severe acute respiratory syndrome coronavirus 2
LTDEMIAQY

SEQ ID NO: 376 - protein, 9 aa, Severe acute respiratory syndrome coronavirus 2
QYIKWPWYI

SEQ ID NO: 377 - protein, 9 aa, Severe acute respiratory syndrome coronavirus 2
VLKGVKLHY

SEQ ID NO: 378 - protein, 9 aa, Severe acute respiratory syndrome coronavirus 2
VTYVPAQEK

SEQ ID NO: 379 - protein, 9 aa, Severe acute respiratory syndrome coronavirus 2
KCYGVSPTK

SEQ ID NO: 380 - protein, 9 aa, Severe acute respiratory syndrome coronavirus 2
SPRRARSVA

SEQ ID NO: 381 - protein, 9 aa, Severe acute respiratory syndrome coronavirus 2
YEQYIKWPW

SEQ ID NO: 382 - protein, 9 aa, Severe acute respiratory syndrome coronavirus 2
AEIRASANL

SEQ ID NO: 383 - protein, 9 aa, Severe acute respiratory syndrome coronavirus 2
GVYFASTEK

SEQ ID NO: 384 - protein, 9 aa, Severe acute respiratory syndrome coronavirus 2
GVYYHKNNK

SEQ ID NO: 385 - protein, 9 aa, Severe acute respiratory syndrome coronavirus 2
MIAQYTSAL

SEQ ID NO: 386 - protein, 9 aa, Severe acute respiratory syndrome coronavirus 2
NSASFSTFK

SEQ ID NO: 387 - protein, 9 aa, Severe acute respiratory syndrome coronavirus 2
NYNYLYRLF

SEQ ID NO: 388 - protein, 9 aa, Severe acute respiratory syndrome coronavirus 2
ADAGFIKQY

SEQ ID NO: 389 - protein, 9 aa, Severe acute respiratory syndrome coronavirus 2
ALDPLSETK

SEQ ID NO: 390 - protein, 9 aa, Severe acute respiratory syndrome coronavirus 2
AYSNNSIAI

SEQ ID NO: 391 - protein, 9 aa, Severe acute respiratory syndrome coronavirus 2
DAVRDPQTL

SEQ ID NO: 392 - protein, 9 aa, Severe acute respiratory syndrome coronavirus 2
EILPVSMTK

SEQ ID NO: 393 - protein, 9 aa, Severe acute respiratory syndrome coronavirus 2
ETKCTLKSF

SEQ ID NO: 394 - protein, 9 aa, Severe acute respiratory syndrome coronavirus 2
EVFAQVKQI

SEQ ID NO: 395 - protein, 9 aa, Severe acute respiratory syndrome coronavirus 2
EYVSQPFLM

SEQ ID NO: 396 - protein, 9 aa, Severe acute respiratory syndrome coronavirus 2
FAMQMAYRF

SEQ ID NO: 397 - protein, 9 aa, Severe acute respiratory syndrome coronavirus 2
FASVYAWNR

SEQ ID NO: 398 - protein, 9 aa, Severe acute respiratory syndrome coronavirus 2
FERDISTEI

SEQ ID NO: 399 - protein, 9 aa, Severe acute respiratory syndrome coronavirus 2
FPQSAPHGV

SEQ ID NO: 400 - protein, 9 aa, Severe acute respiratory syndrome coronavirus 2
FTISVTTEI

SEQ ID NO: 401 - protein, 9 aa, Severe acute respiratory syndrome coronavirus 2
FVIRGDEVR

SEQ ID NO: 402 - protein, 9 aa, Severe acute respiratory syndrome coronavirus 2
FVSNGTHWF

SEQ ID NO: 403 - protein, 9 aa, Severe acute respiratory syndrome coronavirus 2
GAAAYYVGY

SEQ ID NO: 404 - protein, 9 aa, Severe acute respiratory syndrome coronavirus 2
GEVFNATRF

SEQ ID NO: 405 - protein, 9 aa, Severe acute respiratory syndrome coronavirus 2
HLMSFPQSA

SEQ ID NO: 406 - protein, 9 aa, Severe acute respiratory syndrome coronavirus 2
HVSGTNGTK

SEQ ID NO: 407 - protein, 9 aa, Severe acute respiratory syndrome coronavirus 2
HWFVTQRNF

SEQ ID NO: 408 - protein, 9 aa, Severe acute respiratory syndrome coronavirus 2
IAIPTNFTI

SEQ ID NO: 409 - protein, 9 aa, Severe acute respiratory syndrome coronavirus 2
IANQFNSAI

SEQ ID NO: 410 - protein, 9 aa, Severe acute respiratory syndrome coronavirus 2
INITRFQTL

SEQ ID NO: 411 - protein, 9 aa, Severe acute respiratory syndrome coronavirus 2
IPFAMQMAY

SEQ ID NO: 412 - protein, 9 aa, Severe acute respiratory syndrome coronavirus 2
IPTNFTISV

SEQ ID NO: 413 - protein, 9 aa, Severe acute respiratory syndrome coronavirus 2
IYQTSNFRV

SEQ ID NO: 414 - protein, 9 aa, Severe acute respiratory syndrome coronavirus 2
KIADYNYKL

SEQ ID NO: 415 - protein, 9 aa, Severe acute respiratory syndrome coronavirus 2
KIYSKHTPI

SEQ ID NO: 416 - protein, 9 aa, Severe acute respiratory syndrome coronavirus 2
KSNLKPFER

SEQ ID NO: 417 - protein, 9 aa, Severe acute respiratory syndrome coronavirus 2
KVFRSSVLH

SEQ ID NO: 418 - protein, 9 aa, Severe acute respiratory syndrome coronavirus 2
LGAENSVAY

SEQ ID NO: 419 - protein, 9 aa, Severe acute respiratory syndrome coronavirus 2
LPFNDGVYF

SEQ ID NO: 420 - protein, 9 aa, Severe acute respiratory syndrome coronavirus 2
LPLVSSQCV

SEQ ID NO: 421 - protein, 9 aa, Severe acute respiratory syndrome coronavirus 2
LPPLLTDEM

SEQ ID NO: 422 - protein, 9 aa, Severe acute respiratory syndrome coronavirus 2
NASVVNIQK

SEQ ID NO: 423 - protein, 9 aa, Severe acute respiratory syndrome coronavirus 2
NATRFASVY

SEQ ID NO: 424 - protein, 9 aa, Severe acute respiratory syndrome coronavirus 2
NSFTRGVYY

SEQ ID NO: 425 - protein, 9 aa, Severe acute respiratory syndrome coronavirus 2
NSIAIPTNF

SEQ ID NO: 426 - protein, 9 aa, Severe acute respiratory syndrome coronavirus 2
QELGKYEQY

SEQ ID NO: 427 - protein, 9 aa, Severe acute respiratory syndrome coronavirus 2
QIAPGQTGK

SEQ ID NO: 428 - protein, 9 aa, Severe acute respiratory syndrome coronavirus 2
QLTPTWRVY

SEQ ID NO: 429 - protein, 9 aa, Severe acute respiratory syndrome coronavirus 2
QPTESIVRF

SEQ ID NO: 430 - protein, 9 aa, Severe acute respiratory syndrome coronavirus 2
QTNSPRRAR

SEQ ID NO: 431 - protein, 9 aa, Severe acute respiratory syndrome coronavirus 2
RSVASQSII

SEQ ID NO: 432 - protein, 9 aa, Severe acute respiratory syndrome coronavirus 2
RVYSTGSNV

SEQ ID NO: 433 - protein, 9 aa, Severe acute respiratory syndrome coronavirus 2
SANNCTFEY

SEQ ID NO: 434 - protein, 9 aa, Severe acute respiratory syndrome coronavirus 2
SFKEELDKY

SEQ ID NO: 435 - protein, 9 aa, Severe acute respiratory syndrome coronavirus 2
SVYAWNRKR

SEQ ID NO: 436 - protein, 9 aa, Severe acute respiratory syndrome coronavirus 2
TLADAGFIK

SEQ ID NO: 437 - protein, 9 aa, Severe acute respiratory syndrome coronavirus 2
VASQSIIAY

SEQ ID NO: 438 - protein, 9 aa, Severe acute respiratory syndrome coronavirus 2
VFAQVKQIY

SEQ ID NO: 439 - protein, 9 aa, Severe acute respiratory syndrome coronavirus 2
VFKNIDGYF

SEQ ID NO: 440 - protein, 9 aa, Severe acute respiratory syndrome coronavirus 2
VGGNYNYLY

SEQ ID NO: 441 - protein, 9 aa, Severe acute respiratory syndrome coronavirus 2
WFVTQRNFY

SEQ ID NO: 442 - protein, 9 aa, Severe acute respiratory syndrome coronavirus 2
WTAGAAAYY

SEQ ID NO: 443 - protein, 9 aa, Severe acute respiratory syndrome coronavirus 2
WTFGAGAAL

SEQ ID NO: 444 - protein, 9 aa, Severe acute respiratory syndrome coronavirus 2
YGFQPTNGV

SEQ ID NO: 445 - protein, 9 aa, Severe acute respiratory syndrome coronavirus 2
YNYLYRLFR

SEQ ID NO: 446 - protein, 9 aa, Severe acute respiratory syndrome coronavirus 2
YQDVNCTEV

SEQ ID NO: 447 - protein, 9 aa, Severe acute respiratory syndrome coronavirus 2
YYHKNNKSW

SEQ ID NO: 448 - protein, 9 aa, Severe acute respiratory syndrome coronavirus 2
TQDLFLPFF

SEQ ID NO: 449 - protein, 9 aa, Severe acute respiratory syndrome coronavirus 2
FVFLVLLPL

SEQ ID NO: 450 - protein, 9 aa, Severe acute respiratory syndrome coronavirus 2
LVKNKCVNF

SEQ ID NO: 451 - protein, 9 aa, Severe acute respiratory syndrome coronavirus 2
STQDLFLPF

SEQ ID NO: 452 - protein, 9 aa, Severe acute respiratory syndrome coronavirus 2
NIADYNYKL

SEQ ID NO: 453 - protein, 9 aa, Severe acute respiratory syndrome coronavirus 2
NYNYRYRLF

SEQ ID NO: 454 - protein, 9 aa, Severe acute respiratory syndrome coronavirus 2
TIADYNYKL

SEQ ID NO: 455 - protein, 10 aa, Human adenovirus 5
LLYANSAHAL

SEQ ID NO: 456 - protein, 10 aa, Human alphaherpesvirus 1
SSGVVFGTWY

SEQ ID NO: 457 - protein, 10 aa, Human alphaherpesvirus 1
EYVHARWAAF

SEQ ID NO: 458 - protein, 10 aa, Human alphaherpesvirus 1
HTDLHPNNTY

SEQ ID NO: 459 - protein, 10 aa, Human alphaherpesvirus 1
AYLGAFLSVL

SEQ ID NO: 460 - protein, 10 aa, Human alphaherpesvirus 1
FVYTPSPYVF

SEQ ID NO: 461 - protein, 10 aa, Human alphaherpesvirus 1
AYSLLFPAPF

SEQ ID NO: 462 - protein, 10 aa, Human alphaherpesvirus 1
RPTERPRAPA

SEQ ID NO: 463 - protein, 10 aa, Human alphaherpesvirus 1
FTDALGIDEY

SEQ ID NO: 464 - protein, 10 aa, Human alphaherpesvirus 1
SALPTNADLY

SEQ ID NO: 465 - protein, 10 aa, Human alphaherpesvirus 1
LYPDAPPLRL

SEQ ID NO: 466 - protein, 10 aa, Human alphaherpesvirus 2
GFLIAYQPLL

SEQ ID NO: 467 - protein, 10 aa, Human alphaherpesvirus 2
FLVDAIVRVA

SEQ ID NO: 468 - protein, 10 aa, Human alphaherpesvirus 3
PHSVVNPFVK

SEQ ID NO: 469 - protein, 10 aa, Human alphaherpesvirus 3
MILIEGIFFV

SEQ ID NO: 470 - protein, 10 aa, Human betaherpesvirus 5
TPRVTGGGAM

SEQ ID NO: 471 - protein, 10 aa, Human betaherpesvirus 5
AYAQKIFKIL

SEQ ID NO: 472 - protein, 10 aa, Human betaherpesvirus 5
YILEETSVML

SEQ ID NO: 473 - protein, 10 aa, Human betaherpesvirus 5
RRKMMYMCYR

SEQ ID NO: 474 - protein, 10 aa, Human betaherpesvirus 5
TYSQKIFKIL

SEQ ID NO: 475 - protein, 10 aa, Human gammaherpesvirus 4
RPPIFIRRLH

SEQ ID NO: 476 - protein, 10 aa, Human gammaherpesvirus 4
VEITPYKPTW

SEQ ID NO: 477 - protein, 10 aa, Human gammaherpesvirus 4
EENLLDFVRF

SEQ ID NO: 478 - protein, 10 aa, Human gammaherpesvirus 4
LLDFVRFMGV

SEQ ID NO: 479 - protein, 10 aa, Human gammaherpesvirus 4
PYLFWLAAIA

SEQ ID NO: 480 - protein, 10 aa, Human gammaherpesvirus 4
VMSNTLLSAW

SEQ ID NO: 481 - protein, 10 aa, Human gammaherpesvirus 4
QNGALAINTF

SEQ ID NO: 482 - protein, 10 aa, Human gammaherpesvirus 4
DTPLIPLTIF

SEQ ID NO: 483 - protein, 10 aa, Human gammaherpesvirus 8
ATVKTGNIKL

SEQ ID NO: 484 - protein, 10 aa, Human herpesvirus 2
AERQGSPTPA

SEQ ID NO: 485 - protein, 10 aa, Human herpesvirus 3
KPQGQRLIEV

SEQ ID NO: 486 - protein, 10 aa, Human herpesvirus 3
QTDTYTLLGY

SEQ ID NO: 487 - protein, 10 aa, Human herpesvirus 3
ILIEGIFFVS

SEQ ID NO: 488 - protein, 10 aa, Human herpesvirus 3
HVFPHRFISF

SEQ ID NO: 489 - protein, 10 aa, Human herpesvirus 3
RIRLVVPSAL

SEQ ID NO: 490 - protein, 10 aa, Human metapneumovirus
SLILIGITTL

SEQ ID NO: 491 - protein, 10 aa, Human metapneumovirus
KVEGEQHVIK

SEQ ID NO: 492 - protein, 10 aa, Human metapneumovirus
IAPYAGLIMI

SEQ ID NO: 493 - protein, 10 aa, Human metapneumovirus
KPAVGVYHIV

SEQ ID NO: 494 - protein, 10 aa, Human orthopneumovirus
FSECNALGSY

SEQ ID NO: 495 - protein, 10 aa, Human orthopneumovirus
STELNYNHLY

SEQ ID NO: 496 - protein, 10 aa, Human orthopneumovirus
FLTEAIVHSV

SEQ ID NO: 497 - protein, 10 aa, human papillomavirus 31
YVLDLQPEAT

SEQ ID NO: 498 - protein, 10 aa, human papillomavirus 35
FACYDLCIVY

SEQ ID NO: 499 - protein, 10 aa, human papillomavirus 39
FAFSDLYVVY

SEQ ID NO: 500 - protein, 10 aa, human papillomavirus 45
FAFKDLCIVY

SEQ ID NO: 501 - protein, 10 aa, human papillomavirus 51
VAFTEIKIVY

SEQ ID NO: 502 - protein, 10 aa, human papillomavirus 52
YILDLQPETT

SEQ ID NO: 503 - protein, 10 aa, human papillomavirus 58
FVFADLRIVY

SEQ ID NO: 504 - protein, 10 aa, human papillomavirus 73
FAFSDLCIVY

SEQ ID NO: 505 - protein, 10 aa, Human papillomavirus type 16
ILTAFNSSHK

SEQ ID NO: 506 - protein, 10 aa, Human papillomavirus type 16
LTAPTGCIKK

SEQ ID NO: 507 - protein, 10 aa, Human papillomavirus type 16
TLKCLRYRFK

SEQ ID NO: 508 - protein, 10 aa, Human papillomavirus type 16
YYVHEGIRTY

SEQ ID NO: 509 - protein, 10 aa, Human papillomavirus type 16
YICEEASVTV

SEQ ID NO: 510 - protein, 10 aa, Human papillomavirus type 16
LLIRCINCQK

SEQ ID NO: 511 - protein, 10 aa, Human papillomavirus type 16
AVCDKCLKFY

SEQ ID NO: 512 - protein, 10 aa, Human papillomavirus type 16
CVYCKQQLLR

SEQ ID NO: 513 - protein, 10 aa, Human papillomavirus type 16
NPYAVCDKCL

SEQ ID NO: 514 - protein, 10 aa, Human papillomavirus type 16
RPRKLPQLCT

SEQ ID NO: 515 - protein, 10 aa, Human papillomavirus type 16
YAVCDKCLKF

SEQ ID NO: 516 - protein, 10 aa, Human papillomavirus type 16
QYNKPLCDLL

SEQ ID NO: 517 - protein, 10 aa, Human papillomavirus type 16
GIVCPICSQK

SEQ ID NO: 518 - protein, 10 aa, Human papillomavirus type 16
HGDTPTLHEY

SEQ ID NO: 519 - protein, 10 aa, Human papillomavirus type 16
HYNIVTFCCK

SEQ ID NO: 520 - protein, 10 aa, Human papillomavirus type 16
YMLDLQPETT

SEQ ID NO: 521 - protein, 10 aa, Human papillomavirus type 16
TIHDIILECV

SEQ ID NO: 522 - protein, 10 aa, Human papillomavirus type 16
HNIRGRWTGR

SEQ ID NO: 523 - protein, 10 aa, Human papillomavirus type 16
FAFRDLCIVY

SEQ ID NO: 524 - protein, 10 aa, Human papillomavirus type 16
QERPRKLPQL

SEQ ID NO: 525 - protein, 10 aa, Human papillomavirus type 16
RWTGRCMSCC

SEQ ID NO: 526 - protein, 10 aa, Human papillomavirus type 16
SSRTRRETQL

SEQ ID NO: 527 - protein, 10 aa, Human papillomavirus type 16
HPAATHTKAV

SEQ ID NO: 528 - protein, 10 aa, Human papillomavirus type 16
TLLQQYCLYL

SEQ ID NO: 529 - protein, 10 aa, Human parvovirus B19
GLCPHCINVG

SEQ ID NO: 530 - protein, 10 aa, Human parvovirus B19
HAKALKERMV

SEQ ID NO: 531 - protein, 10 aa, Human parvovirus B19
IDTCISATFR

SEQ ID NO: 532 - protein, 10 aa, Human parvovirus B19
LLHTDFEQVM

SEQ ID NO: 533 - protein, 10 aa, Human parvovirus B19
QSALKLAIYK

SEQ ID NO: 534 - protein, 10 aa, Human parvovirus B19
SALKLAIYKA

SEQ ID NO: 535 - protein, 10 aa, Influenza A virus
KLYQNPTTYI

SEQ ID NO: 536 - protein, 10 aa, Influenza A virus
RLYQNPTTYI

SEQ ID NO: 537 - protein, 10 aa, Influenza A virus
KFLPDLYDYK

SEQ ID NO: 538 - protein, 10 aa, Influenza A virus
VLRGFLILGK

SEQ ID NO: 539 - protein, 10 aa, Influenza A virus
GILGFVFTLT

SEQ ID NO: 540 - protein, 10 aa, Influenza A virus
RMVLASTTAK

SEQ ID NO: 541 - protein, 10 aa, Influenza A virus
SFSFGGFTFK

SEQ ID NO: 542 - protein, 10 aa, Severe acute respiratory syndrome coronavirus 2
KNIDGYFKIY

SEQ ID NO: 543 - protein, 10 aa, Severe acute respiratory syndrome coronavirus 2
CMTSCCSCLK

SEQ ID NO: 544 - protein, 10 aa, Severe acute respiratory syndrome coronavirus 2
DSFKEELDKY

SEQ ID NO: 545 - protein, 10 aa, Severe acute respiratory syndrome coronavirus 2
QPYRVVVLSF

SEQ ID NO: 546 - protein, 10 aa, Severe acute respiratory syndrome coronavirus 2
SEPVLKGVKL

SEQ ID NO: 547 - protein, 10 aa, Severe acute respiratory syndrome coronavirus 2
CALDPLSETK

SEQ ID NO: 548 - protein, 10 aa, Severe acute respiratory syndrome coronavirus 2
EILDITPCSF

SEQ ID NO: 549 - protein, 10 aa, Severe acute respiratory syndrome coronavirus 2
FTISVTTEIL

SEQ ID NO: 550 - protein, 10 aa, Severe acute respiratory syndrome coronavirus 2
IGAEHVNNSY

SEQ ID NO: 551 - protein, 10 aa, Severe acute respiratory syndrome coronavirus 2
ILPDPSKPSK

SEQ ID NO: 552 - protein, 10 aa, Severe acute respiratory syndrome coronavirus 2
IYSKHTPINL

SEQ ID NO: 553 - protein, 10 aa, Severe acute respiratory syndrome coronavirus 2
KLPDDFTGCV

SEQ ID NO: 554 - protein, 10 aa, Severe acute respiratory syndrome coronavirus 2
KTSVDCTMYI

SEQ ID NO: 555 - protein, 10 aa, Severe acute respiratory syndrome coronavirus 2
LSSTASALGK

SEQ ID NO: 556 - protein, 10 aa, Severe acute respiratory syndrome coronavirus 2
STQDLFLPFF

SEQ ID NO: 557 - protein, 10 aa, Severe acute respiratory syndrome coronavirus 2
SVLNDILSRL

SEQ ID NO: 558 - protein, 10 aa, Severe acute respiratory syndrome coronavirus 2
TEKSNIIRGW

SEQ ID NO: 559 - protein, 10 aa, Severe acute respiratory syndrome coronavirus 2
WIFGTTLDSK

SEQ ID NO: 560 - protein, 10 aa, Severe acute respiratory syndrome coronavirus 2
YHLMSFPQSA

SEQ ID NO: 561 - protein, 10 aa, Severe acute respiratory syndrome coronavirus 2
TQLNRALTGI

SEQ ID NO: 562 - protein, 10 aa, Severe acute respiratory syndrome coronavirus 2
NESLIDLQEL

SEQ ID NO: 563 - protein, 10 aa, Severe acute respiratory syndrome coronavirus 2
DGVYFASTEK

SEQ ID NO: 564 - protein, 10 aa, Severe acute respiratory syndrome coronavirus 2
DSKVGGNYNY

SEQ ID NO: 565 - protein, 10 aa, Severe acute respiratory syndrome coronavirus 2
EVFAQVKQIY

SEQ ID NO: 566 - protein, 10 aa, Severe acute respiratory syndrome coronavirus 2
FDEDDSEPVL

SEQ ID NO: 567 - protein, 10 aa, Severe acute respiratory syndrome coronavirus 2
FERDISTEIY

SEQ ID NO: 568 - protein, 10 aa, Severe acute respiratory syndrome coronavirus 2
FEYVSQPFLM

SEQ ID NO: 569 - protein, 10 aa, Severe acute respiratory syndrome coronavirus 2
FLPFFSNVTW

SEQ ID NO: 570 - protein, 10 aa, Severe acute respiratory syndrome coronavirus 2
GVFVSNGTHW

SEQ ID NO: 571 - protein, 10 aa, Severe acute respiratory syndrome coronavirus 2
GVYYPDKVFR

SEQ ID NO: 572 - protein, 10 aa, Severe acute respiratory syndrome coronavirus 2
GYLQPRTFLL

SEQ ID NO: 573 - protein, 10 aa, Severe acute respiratory syndrome coronavirus 2
HVTYVPAQEK

SEQ ID NO: 574 - protein, 10 aa, Severe acute respiratory syndrome coronavirus 2
IHADQLTPTW

SEQ ID NO: 575 - protein, 10 aa, Severe acute respiratory syndrome coronavirus 2
KFLPFQQFGR

SEQ ID NO: 576 - protein, 10 aa, Severe acute respiratory syndrome coronavirus 2
KVGGNYNYLY

SEQ ID NO: 577 - protein, 10 aa, Severe acute respiratory syndrome coronavirus 2
LPIGINITRF

SEQ ID NO: 578 - protein, 10 aa, Severe acute respiratory syndrome coronavirus 2
NTSNQVAVLY

SEQ ID NO: 579 - protein, 10 aa, Severe acute respiratory syndrome coronavirus 2
NVYADSFVIR

SEQ ID NO: 580 - protein, 10 aa, Severe acute respiratory syndrome coronavirus 2
QIPFAMQMAY

SEQ ID NO: 581 - protein, 10 aa, Severe acute respiratory syndrome coronavirus 2
QYIKWPWYIW

SEQ ID NO: 582 - protein, 10 aa, Severe acute respiratory syndrome coronavirus 2
RASANLAATK

SEQ ID NO: 583 - protein, 10 aa, Severe acute respiratory syndrome coronavirus 2
RFASVYAWNR

SEQ ID NO: 584 - protein, 10 aa, Severe acute respiratory syndrome coronavirus 2
RVYSTGSNVF

SEQ ID NO: 585 - protein, 10 aa, Severe acute respiratory syndrome coronavirus 2
SETKCTLKSF

SEQ ID NO: 586 - protein, 10 aa, Severe acute respiratory syndrome coronavirus 2
STGSNVFQTR

SEQ ID NO: 587 - protein, 10 aa, Severe acute respiratory syndrome coronavirus 2
SVASQSIIAY

SEQ ID NO: 588 - protein, 10 aa, Severe acute respiratory syndrome coronavirus 2
SWMESEFRVY

SEQ ID NO: 589 - protein, 10 aa, Severe acute respiratory syndrome coronavirus 2
TECSNLLLQY

SEQ ID NO: 590 - protein, 10 aa, Severe acute respiratory syndrome coronavirus 2
TPCSFGGVSV

SEQ ID NO: 591 - protein, 10 aa, Severe acute respiratory syndrome coronavirus 2
VFVSNGTHWF

SEQ ID NO: 592 - protein, 10 aa, Severe acute respiratory syndrome coronavirus 2
VYSSANNCTF

SEQ ID NO: 593 - protein, 10 aa, Severe acute respiratory syndrome coronavirus 2
YTNSFTRGVY

SEQ ID NO: 594 - protein, 10 aa, Severe acute respiratory syndrome coronavirus 2
IYKTPPIKDF

SEQ ID NO: 595 - protein, 10 aa, Severe acute respiratory syndrome coronavirus 2
LLTDEMIAQY

SEQ ID NO: 596 - protein, 10 aa, Severe acute respiratory syndrome coronavirus 2
SYFIASFRLF

SEQ ID NO: 597 - protein, 11 aa, Human alphaherpesvirus 3
EITDTIDKFGK

SEQ ID NO: 598 - protein, 11 aa, Human alphaherpesvirus 3
LPEGMDPFAEK

SEQ ID NO: 599 - protein, 11 aa, Human alphaherpesvirus 3
ARLCDLPATPK

SEQ ID NO: 600 - protein, 11 aa, Human betaherpesvirus 5
LQRGPQYSEHP

SEQ ID NO: 601 - protein, 11 aa, Human betaherpesvirus 5
PSQEPMSIYVY

SEQ ID NO: 602 - protein, 11 aa, Human betaherpesvirus 5
QEFFWDANDIY

SEQ ID NO: 603 - protein, 11 aa, Human betaherpesvirus 5
QYDPVAALFFF

SEQ ID NO: 604 - protein, 11 aa, Human betaherpesvirus 5
RLTVSGLAWTR

SEQ ID NO: 605 - protein, 11 aa, Human betaherpesvirus 5
RNLVPMVATVQ

SEQ ID NO: 606 - protein, 11 aa, Human betaherpesvirus 5
TPRVTGGGAMA

SEQ ID NO: 607 - protein, 11 aa, Human betaherpesvirus 5
VFPTKDVALRH

SEQ ID NO: 608 - protein, 11 aa, Human betaherpesvirus 5
VLCPKNMIIKP

SEQ ID NO: 609 - protein, 11 aa, Human betaherpesvirus 5
YSEHPTFTSQY

SEQ ID NO: 610 - protein, 11 aa, Human betaherpesvirus 5
YYTSAFVFPTK

SEQ ID NO: 611 - protein, 11 aa, Human betaherpesvirus 5
QYTPDSTPCHR

SEQ ID NO: 612 - protein, 11 aa, Human betaherpesvirus 5
RPHERNGFTVL

SEQ ID NO: 613 - protein, 11 aa, Human betaherpesvirus 5
AKARAKKDELR

SEQ ID NO: 614 - protein, 11 aa, Human betaherpesvirus 5
CYVLEETSVML

SEQ ID NO: 615 - protein, 11 aa, Human betaherpesvirus 5
EFCRVLCCYVL

SEQ ID NO: 616 - protein, 11 aa, Human betaherpesvirus 5
EQVTEDCNENP

SEQ ID NO: 617 - protein, 11 aa, Human betaherpesvirus 5
ESLKTFEQVTE

SEQ ID NO: 618 - protein, 11 aa, Human betaherpesvirus 5
FPKTTNGCSQA

SEQ ID NO: 619 - protein, 11 aa, Human betaherpesvirus 5
GKSTHPMVTRS

SEQ ID NO: 620 - protein, 11 aa, Human betaherpesvirus 5
KDELRRKMMYM

SEQ ID NO: 621 - protein, 11 aa, Human betaherpesvirus 5
KNSAFPKTTNG

SEQ ID NO: 622 - protein, 11 aa, Human betaherpesvirus 5
KQIKVRVDMVR

SEQ ID NO: 623 - protein, 11 aa, Human betaherpesvirus 5
MAYAQKIFKIL

SEQ ID NO: 624 - protein, 11 aa, Human betaherpesvirus 5
NIEFFTKNSAF

SEQ ID NO: 625 - protein, 11 aa, Human betaherpesvirus 5
SVMKRRIEEIC

SEQ ID NO: 626 - protein, 11 aa, Human gammaherpesvirus 4
YPRNPTEQGNI

SEQ ID NO: 627 - protein, 11 aa, Human gammaherpesvirus 4
HPVGEADYFEY

SEQ ID NO: 628 - protein, 11 aa, Human gammaherpesvirus 4
QPRAPIRPIPT

SEQ ID NO: 629 - protein, 11 aa, Human gammaherpesvirus 4
SSCSSCPLSKI

SEQ ID NO: 630 - protein, 11 aa, Human gammaherpesvirus 4
EPLPQGQLTAY

SEQ ID NO: 631 - protein, 11 aa, Human herpesvirus 3
AVYENPLSVEK

SEQ ID NO: 632 - protein, 11 aa, Human parvovirus B19
TEADVQQWLTW

SEQ ID NO: 633 - protein, 11 aa, Human polyomavirus 1
FFAVGGDPLEM

SEQ ID NO: 634 - protein, 11 aa, Human respiratory syncytial virus A2
IAVGLLLYCKA

SEQ ID NO: 635 - protein, 11 aa, Influenza A virus
TIAMELIRMIK

SEQ ID NO: 636 - protein, 11 aa, Severe acute respiratory syndrome coronavirus 2
EILDITPCSFG

SEQ ID NO: 637 - protein, 11 aa, Severe acute respiratory syndrome coronavirus 2
AQALNTLVKQL

SEQ ID NO: 638 - protein, 11 aa, Severe acute respiratory syndrome coronavirus 2
FCNDPFLGVYY

SEQ ID NO: 639 - protein, 11 aa, Severe acute respiratory syndrome coronavirus 2
FPQSAPHGVVF

SEQ ID NO: 640 - protein, 11 aa, Severe acute respiratory syndrome coronavirus 2
KSWMESEFRVY

SEQ ID NO: 641 - protein, 11 aa, Severe acute respiratory syndrome coronavirus 2
LQIPFAMQMAY

SEQ ID NO: 642 - protein, 11 aa, Severe acute respiratory syndrome coronavirus 2
NYNYLYRLFRK

SEQ ID NO: 643 - protein, 11 aa, Severe acute respiratory syndrome coronavirus 2
YEQYIKWPWYI

SEQ ID NO: 644 - protein, 11 aa, Severe acute respiratory syndrome coronavirus 2
IPIGAGICASY

SEQ ID NO: 645 - protein, 11 aa, Severe acute respiratory syndrome coronavirus 2
KPFERDISTEI

SEQ ID NO: 646 - protein, 11 aa, Severe acute respiratory syndrome coronavirus 2
QEVFAQVKQIY

SEQ ID NO: 647 - protein, 11 aa, Severe acute respiratory syndrome coronavirus 2
RFPNITNLCPF

SEQ ID NO: 648 - protein, 11 aa, Severe acute respiratory syndrome coronavirus 2
YECDIPIGAGI

SEQ ID NO: 649 - protein, 11 aa, Severe acute respiratory syndrome coronavirus 2
YENQKLIANQF

SEQ ID NO: 650 - protein, 11 aa, Severe acute respiratory syndrome coronavirus 2
YYVGYLQPRTF

SEQ ID NO: 651 - protein, 11 aa, Severe acute respiratory syndrome coronavirus 2
SFELLHAPATV

SEQ ID NO: 652 - protein, 11 aa, Severe acute respiratory syndrome coronavirus 2
TYVPAQEKNFT

SEQ ID NO: 653 - protein, 11 aa, Epstein Barr Virus
HPVGDADYFEY

SEQ ID NO: 654 - protein, 11 aa, Epstein Barr Virus
HPVGQADYFEY

SEQ ID NO: 655 - protein, 11 aa, Severe acute respiratory syndrome coronavirus 2
RVAGDSGFAAY

SEQ ID NO: 656 - protein, 10 aa, Human betaherpesvirus 5
EFFWDANDIY

SEQ ID NO: 657 - protein, 10 aa, Human betaherpesvirus 5
RPHERNGFTV

SEQ ID NO: 658 - protein, 10 aa, Influenza A virus
LPRRSGAAGA

SEQ ID NO: 659 - protein, 10 aa, Severe acute respiratory syndrome coronavirus 2
ALQIPFAMQM

SEQ ID NO: 660 - protein, 10 aa, Severe acute respiratory syndrome coronavirus 2
QELIRQGTDY

SEQ ID NO: 661 - protein, 10 aa, Severe acute respiratory syndrome coronavirus 2
RARSVASQSI

SEQ ID NO: 662 - protein, 10 aa, Severe acute respiratory syndrome coronavirus 2
RLITGRLQSL

SEQ ID NO: 663 - protein, 10 aa, Severe acute respiratory syndrome coronavirus 2
TLATHGLAAV

SEQ ID NO: 664 - protein, 10 aa, Severe acute respiratory syndrome coronavirus 2
TTDPSFLGRY

SEQ ID NO: 665 - protein, 10 aa, Severe acute respiratory syndrome coronavirus 2
VENPHLMGWD

SEQ ID NO: 666 - protein, 10 aa, Severe acute respiratory syndrome coronavirus 2
YPDKVFRSSV

SEQ ID NO: 667 - protein, 9 aa, Human betaherpesvirus 5
AYAQKIFKI

SEQ ID NO: 668 - protein, 9 aa, Human betaherpesvirus 5
CRVLCCYVL

SEQ ID NO: 669 - protein, 9 aa, Human betaherpesvirus 5
CVETMCNEY

SEQ ID NO: 670 - protein, 9 aa, Human betaherpesvirus 5
DEEDAIAAY

SEQ ID NO: 671 - protein, 9 aa, Human betaherpesvirus 5
FRCPRRFCF

SEQ ID NO: 672 - protein, 9 aa, Human betaherpesvirus 5
KLGGALQAK

SEQ ID NO: 673 - protein, 9 aa, Human betaherpesvirus 5
MLNIPSINV

SEQ ID NO: 674 - protein, 9 aa, Human betaherpesvirus 5
VMAPRTLIL

SEQ ID NO: 675 - protein, 9 aa, Human betaherpesvirus 5
YILEETSVM

SEQ ID NO: 676 - protein, 9 aa, Human papillomavirus type 16
MLDLQPETT

SEQ ID NO: 677 - protein, 9 aa, Influenza A virus
CTELKLSDY

SEQ ID NO: 678 - protein, 9 aa, Influenza A virus
FEDLRVLSF

SEQ ID NO: 679 - protein, 9 aa, Severe acute respiratory syndrome coronavirus 2
ALSKGVHFV

SEQ ID NO: 680 - protein, 9 aa, Severe acute respiratory syndrome coronavirus 2
ALWEIQQVV

SEQ ID NO: 681 - protein, 9 aa, Severe acute respiratory syndrome coronavirus 2
DTDFVNEFY

SEQ ID NO: 682 - protein, 9 aa, Severe acute respiratory syndrome coronavirus 2
FIAGLIAIV

SEQ ID NO: 683 - protein, 9 aa, Severe acute respiratory syndrome coronavirus 2
FTSDYYQLY

SEQ ID NO: 684 - protein, 9 aa, Severe acute respiratory syndrome coronavirus 2
KQIYKTPPI

SEQ ID NO: 685 - protein, 9 aa, Severe acute respiratory syndrome coronavirus 2
KTFPPTEPK

SEQ ID NO: 686 - protein, 9 aa, Severe acute respiratory syndrome coronavirus 2
LLLDRLNQL

SEQ ID NO: 687 - protein, 9 aa, Severe acute respiratory syndrome coronavirus 2
LLYDANYFL

SEQ ID NO: 688 - protein, 9 aa, Severe acute respiratory syndrome coronavirus 2
MLAKALRKV

SEQ ID NO: 689 - protein, 9 aa, Severe acute respiratory syndrome coronavirus 2
MQLFFSYFA

SEQ ID NO: 690 - protein, 9 aa, Severe acute respiratory syndrome coronavirus 2
PTDNYITTY

SEQ ID NO: 691 - protein, 9 aa, Severe acute respiratory syndrome coronavirus 2
SPRWYFYYL

SEQ ID NO: 692 - protein, 9 aa, Severe acute respiratory syndrome coronavirus 2
VYFLQSINF

SEQ ID NO: 693 - protein, 9 aa, Severe acute respiratory syndrome coronavirus 2
VYIGDPAQL

SEQ ID NO: 694 - protein, 8 aa, Human betaherpesvirus 5
FPTKDVAL

SEQ ID NO: 695 - protein, 8 aa, Human betaherpesvirus 5
NEGVKAAW

SEQ ID NO: 696 - protein, 9 aa, Human betaherpesvirus 5
DYNFVKQLF

SEQ ID NO: 697 - protein, 9 aa, Human betaherpesvirus 5
TYPVLEEMF

SEQ ID NO: 698 - protein, 9 aa, Human betaherpesvirus 5
RYSIFFDYM

SEQ ID NO: 699 - protein, 9 aa, Human betaherpesvirus 5
TYSAGIVQI

SEQ ID NO: 700 - protein, 9 aa, Human betaherpesvirus 5
VYALPLKML

SEQ ID NO: 701 - protein, 9 aa, Human betaherpesvirus 5
GPISGHVLK

SEQ ID NO: 702 - protein, 9 aa, Homo sapiens
SQKTYQGSY

SEQ ID NO: 703 - protein, 12 aa, Homo sapiens
KEFTVSGNILTI

SEQ ID NO: 704 - protein, 9 aa, Homo sapiens
MVKISGGPR

SEQ ID NO: 705 - protein, 11 aa, Homo sapiens
AEEAAGIGILT

SEQ ID NO: 706 - protein, 9 aa, Homo sapiens
DPARYEFLW

SEQ ID NO: 707 - protein, 9 aa, Homo sapiens
DPARYEFLW

SEQ ID NO: 708 - protein, 10 aa, Homo sapiens
REPVTKAEML

SEQ ID NO: 709 - protein, 10 aa, Homo sapiens
REPVTKAEML

SEQ ID NO: 710 - protein, 10 aa, Homo sapiens
REPVTKAEML

SEQ ID NO: 711 - protein, 8 aa, Homo sapiens
SESLKMIF

SEQ ID NO: 712 - protein, 10 aa, Homo sapiens
REPVTKAEML

SEQ ID NO: 713 - protein, 12 aa, Homo sapiens
CTACRWKKACQR

SEQ ID NO: 714 - protein, 8 aa, Homo sapiens
CYMEAVAL

SEQ ID NO: 715 - protein, 10 aa, Homo sapiens
LAAQERRVPR

SEQ ID NO: 716 - protein, 10 aa, Homo sapiens
LAAQERRVPR

SEQ ID NO: 717 - protein, 10 aa, Homo sapiens
ASGPGGGAPR

SEQ ID NO: 718 - protein, 10 aa, Homo sapiens
LAAQERRVPR

SEQ ID NO: 719 - protein, 10 aa, Homo sapiens
LAAQERRVPR

SEQ ID NO: 720 - protein, 9 aa, Homo sapiens
MSLQRQFLR

SEQ ID NO: 721 - protein, 9 aa, Homo sapiens
LLGPGRPYR

SEQ ID NO: 722 - protein, 9 aa, Homo sapiens
LLGPGRPYR

SEQ ID NO: 723 - protein, 9 aa, Homo sapiens
LLGPGRPYR

SEQ ID NO: 724 - protein, 9 aa, Homo sapiens
SHETVIIEL

SEQ ID NO: 725 - protein, 10 aa, Homo sapiens
LHHAFVDSIF

SEQ ID NO: 726 - protein, 8 aa, Homo sapiens
QQITKTEV

SEQ ID NO: 727 - protein, 9 aa, Homo sapiens
WQYFFPVIF

SEQ ID NO: 728 - protein, 9 aa, Homo sapiens
FATPMEAEL

SEQ ID NO: 729 - protein, 9 aa, Homo sapiens
YYWPRPRRY

SEQ ID NO: 730 - protein, 9 aa, Homo sapiens
QGQHFLQKV

SEQ ID NO: 731 - protein, 10 aa, Homo sapiens
MEVDPIGHLY

SEQ ID NO: 732 - protein, 9 aa, Homo sapiens
AELVHFLLL

SEQ ID NO: 733 - protein, 9 aa, Homo sapiens
SRFGGAVVR

SEQ ID NO: 734 - protein, 9 aa, Homo sapiens
AARAVFLAL

SEQ ID NO: 735 - protein, 9 aa, Homo sapiens
SAYGEPRKL

SEQ ID NO: 736 - protein, 9 aa, Homo sapiens
ISGGPRISY

SEQ ID NO: 737 - protein, 9 aa, Homo sapiens
GADGVGKSA

SEQ ID NO: 738 - protein, 10 aa, Homo sapiens
GADGVGKSAL

SEQ ID NO: 739 - protein, 8 aa, Homo sapiens
SNDGPTLI

SEQ ID NO: 740 - protein, 9 aa, Homo sapiens
ANDPIFVVL

SEQ ID NO: 741 - protein, 9 aa, Homo sapiens
QCSGNFMGF

SEQ ID NO: 742 - protein, 9 aa, Homo sapiens
RTKQLYPEW

SEQ ID NO: 743 - protein, 9 aa, Homo sapiens
ETVSEQSNV

SEQ ID NO: 744 - protein, 9 aa, Homo sapiens
EAFIQPITR

SEQ ID NO: 745 - protein, 9 aa, Homo sapiens
ELVRRILSR

SEQ ID NO: 746 - protein, 10 aa, Homo sapiens
EVYDGREHSA

SEQ ID NO: 747 - protein, 10 aa, Homo sapiens
TVSGNILTIR

SEQ ID NO: 748 - protein, 10 aa, Homo sapiens
EVISCKLIKR

SEQ ID NO: 749 - protein, 10 aa, Homo sapiens
HTMEVTVYHR

SEQ ID NO: 750 - protein, 11 aa, Homo sapiens
ITKKVADLVGF

SEQ ID NO: 751 - protein, 9 aa, Homo sapiens
ASSTLYLVF

SEQ ID NO: 752 - protein, 9 aa, Homo sapiens
SAFPTTINF

SEQ ID NO: 753 - protein, 9 aa, Homo sapiens
MPFATPMEA

SEQ ID NO: 754 - protein, 11 aa, Homo sapiens
FATPMEAELAR

SEQ ID NO: 755 - protein, 9 aa, Homo sapiens
CILGKLFTK

SEQ ID NO: 756 - protein, 9 aa, Homo sapiens
AVCPWTWLR

SEQ ID NO: 757 - protein, 9 aa, Homo sapiens
KTLTSVFQK

SEQ ID NO: 758 - protein, 10 aa, Homo sapiens
ATFLGSLTWK

SEQ ID NO: 759 - protein, 9 aa, Homo sapiens
ALNFPGSQK

SEQ ID NO: 760 - protein, 9 aa, Homo sapiens
RQKRILVNL

SEQ ID NO: 761 - protein, 9 aa, Homo sapiens
GFKQSSKAL

SEQ ID NO: 762 - protein, 9 aa, Homo sapiens
LSRLSNRLL

SEQ ID NO: 763 - protein, 9 aa, Homo sapiens
LSRLSNRLL

SEQ ID NO: 764 - protein, 9 aa, Homo sapiens
RPHVPESAF

SEQ ID NO: 765 - protein, 10 aa, Homo sapiens
QRPYGYDQIM

SEQ ID NO: 766 - protein, 9 aa, Homo sapiens
APRGVRMAV

SEQ ID NO: 767 - protein, 9 aa, Homo sapiens
APRGVRMAV

SEQ ID NO: 768 - protein, 9 aa, Homo sapiens
RVRFFFPSL

SEQ ID NO: 769 - protein, 13 aa, Homo sapiens
APRGPHGGAASGL

SEQ ID NO: 770 - protein, 9 aa, Homo sapiens
SSPGCQPPA

SEQ ID NO: 771 - protein, 9 aa, Homo sapiens
SPRWWPTCL

SEQ ID NO: 772 - protein, 10 aa, Homo sapiens
SPSSNRIRNT

SEQ ID NO: 773 - protein, 9 aa, Homo sapiens
LPRWPPPQL

SEQ ID NO: 774 - protein, 9 aa, Homo sapiens
SAYGEPRKL

SEQ ID NO: 775 - protein, 9 aa, Homo sapiens
LAMPFATPM

SEQ ID NO: 776 - protein, 9 aa, Homo sapiens
FRSGLDSYV

SEQ ID NO: 777 - protein, 8 aa, Homo sapiens
YRPRPRRY

SEQ ID NO: 778 - protein, 9 aa, Homo sapiens
ARGPESRLL

SEQ ID NO: 779 - protein, 9 aa, Homo sapiens
SYLDSGIHF

SEQ ID NO: 780 - protein, 9 aa, Homo sapiens
NYNNFYRFL

SEQ ID NO: 781 - protein, 10 aa, Homo sapiens
EYSKECLKEF

SEQ ID NO: 782 - protein, 9 aa, Homo sapiens
EYLSLSDKI

SEQ ID NO: 783 - protein, 10 aa, Homo sapiens
RYCNLEGPPI

SEQ ID NO: 784 - protein, 9 aa, Homo sapiens
EYLQLVFGI

SEQ ID NO: 785 - protein, 9 aa, Homo sapiens
TFPDLESEF

SEQ ID NO: 786 - protein, 9 aa, Homo sapiens
VAELVHFLL

SEQ ID NO: 787 - protein, 9 aa, Homo sapiens
NYKRCFPVI

SEQ ID NO: 788 - protein, 9 aa, Homo sapiens
NYKRCFPVI

SEQ ID NO: 789 - protein, 11 aa, Homo sapiens
YLAMPFATPME

SEQ ID NO: 790 - protein, 9 aa, Homo sapiens
LYATVIHDI

SEQ ID NO: 791 - protein, 9 aa, Homo sapiens
TYACFVSNL

SEQ ID NO: 792 - protein, 10 aa, Homo sapiens
QYSWFVNGTF

SEQ ID NO: 793 - protein, 9 aa, Homo sapiens
VYFFLPDHL

SEQ ID NO: 794 - protein, 9 aa, Homo sapiens
LYSACFWWL

SEQ ID NO: 795 - protein, 11 aa, Homo sapiens
IYMDGTADFSF

SEQ ID NO: 796 - protein, 9 aa, Homo sapiens
AFLPWHRLF

SEQ ID NO: 797 - protein, 9 aa, Homo sapiens
KFLDALISL

SEQ ID NO: 798 - protein, 9 aa, Homo sapiens
RYQLDPKFI

SEQ ID NO: 799 - protein, 9 aa, Homo sapiens
KYDCFLHPF

SEQ ID NO: 800 - protein, 9 aa, Homo sapiens
KYVGIEREM

SEQ ID NO: 801 - protein, 9 aa, Homo sapiens
EYILSLEEL

SEQ ID NO: 802 - protein, 9 aa, Homo sapiens
TYLPTNASL

SEQ ID NO: 803 - protein, 9 aa, Homo sapiens
NYGIYKQDL

SEQ ID NO: 804 - protein, 9 aa, Homo sapiens
TCQPTCRSL

SEQ ID NO: 805 - protein, 10 aa, Homo sapiens
LYVDSLFFLC

SEQ ID NO: 806 - protein, 9 aa, Homo sapiens
NYARTEDFF

SEQ ID NO: 807 - protein, 9 aa, Homo sapiens
NSQPVWLCL

SEQ ID NO: 808 - protein, 9 aa, Homo sapiens
CMTWNQMNL

SEQ ID NO: 809 - protein, 9 aa, Homo sapiens
FPSDSWCYF

SEQ ID NO: 810 - protein, 9 aa, Homo sapiens
VVVGAVGVG

SEQ ID NO: 811 - protein, 9 aa, Homo sapiens
EADPTGHSY

SEQ ID NO: 812 - protein, 9 aa, Homo sapiens
EVDPIGHLY

SEQ ID NO: 813 - protein, 9 aa, Homo sapiens
EVDPIGHVY

SEQ ID NO: 814 - protein, 11 aa, Homo sapiens
MPFATPMEAEL

SEQ ID NO: 815 - protein, 10 aa, Homo sapiens
VPLDCVLYRY

SEQ ID NO: 816 - protein, 9 aa, Homo sapiens
LPHSSSHWL

SEQ ID NO: 817 - protein, 10 aa, Homo sapiens
EAAGIGILTV

SEQ ID NO: 818 - protein, 9 aa, Homo sapiens
LPSSADVEF

SEQ ID NO: 819 - protein, 12 aa, Homo sapiens
TPRLPSSADVEF

SEQ ID NO: 820 - protein, 14 aa, Homo sapiens
LPAVVGLSPGEQEY

SEQ ID NO: 821 - protein, 9 aa, Homo sapiens
AEPINIQTW

SEQ ID NO: 822 - protein, 9 aa, Homo sapiens
EEKLIVVLF

SEQ ID NO: 823 - protein, 11 aa, Homo sapiens
SELFRSGLDSY

SEQ ID NO: 824 - protein, 9 aa, Homo sapiens
KELEGILLL

SEQ ID NO: 825 - protein, 10 aa, Homo sapiens
KEADPTGHSY

SEQ ID NO: 826 - protein, 10 aa, Homo sapiens
MEVDPIGHLY

SEQ ID NO: 827 - protein, 9 aa, Homo sapiens
SESIKKKVL

SEQ ID NO: 828 - protein, 10 aa, Homo sapiens
SEIWRDIDFD

SEQ ID NO: 829 - protein, 10 aa, Homo sapiens
KILDAVVAQK

SEQ ID NO: 830 - protein, 10 aa, Homo sapiens
TLDWLLQTPK

SEQ ID NO: 831 - protein, 9 aa, Homo sapiens
KINKNPKYK

SEQ ID NO: 832 - protein, 9 aa, Homo sapiens
KIFSEVTLK

SEQ ID NO: 833 - protein, 9 aa, Homo sapiens
SLFRAVITK

SEQ ID NO: 834 - protein, 9 aa, Homo sapiens
HLFGYSWYK

SEQ ID NO: 835 - protein, 9 aa, Homo sapiens
LIYRRRLMK

SEQ ID NO: 836 - protein, 10 aa, Homo sapiens
IALNFPGSQK

SEQ ID NO: 837 - protein, 9 aa, Homo sapiens
ALLAVGATK

SEQ ID NO: 838 - protein, 9 aa, Homo sapiens
RSYVPLAHR

SEQ ID NO: 839 - protein, 9 aa, Homo sapiens
ALNFPGSQK

SEQ ID NO: 840 - protein, 9 aa, Homo sapiens
PLLENVISK

SEQ ID NO: 841 - protein, 10 aa, Homo sapiens
NTYASPRFKF

SEQ ID NO: 842 - protein, 9 aa, Homo sapiens
VLRENTSPK

SEQ ID NO: 843 - protein, 12 aa, Homo sapiens
SLFPNSPKWTSK

SEQ ID NO: 844 - protein, 10 aa, Homo sapiens
GLASFKSFLK

SEQ ID NO: 845 - protein, 10 aa, Homo sapiens
RAGLQVRKNK

SEQ ID NO: 846 - protein, 10 aa, Homo sapiens
YVDFREYEYY

SEQ ID NO: 847 - protein, 11 aa, Homo sapiens
FLEGNEVGKTY

SEQ ID NO: 848 - protein, 10 aa, Homo sapiens
ILDTAGREEY

SEQ ID NO: 849 - protein, 9 aa, Homo sapiens
YTDFHCQYV

SEQ ID NO: 850 - protein, 9 aa, Homo sapiens
EADPTGHSY

SEQ ID NO: 851 - protein, 9 aa, Homo sapiens
EVDPIGHLY

SEQ ID NO: 852 - protein, 9 aa, Homo sapiens
EVDPASNTY

SEQ ID NO: 853 - protein, 9 aa, Homo sapiens
ILDSSEEDK

SEQ ID NO: 854 - protein, 9 aa, Homo sapiens
KCDICTDEY

SEQ ID NO: 855 - protein, 11 aa, Homo sapiens
SSDYVIPIGTY

SEQ ID NO: 856 - protein, 10 aa, Homo sapiens
RSDSGQQARY

SEQ ID NO: 857 - protein, 11 aa, Homo sapiens
TSEKRPFMCAY

SEQ ID NO: 858 - protein, 9 aa, Homo sapiens
RVRFFFPSL

SEQ ID NO: 859 - protein, 9 aa, Homo sapiens
VRIGHLYIL

SEQ ID NO: 860 - protein, 9 aa, Homo sapiens
VRIGHLYIL

SEQ ID NO: 861 - protein, 9 aa, Homo sapiens
EGDCAPEEK

SEQ ID NO: 862 - protein, 9 aa, Homo sapiens
EGDCAPEEK

SEQ ID NO: 863 - protein, 9 aa, Homo sapiens
EGDCAPEEK

SEQ ID NO: 864 - protein, 9 aa, Homo sapiens
EGDCAPEEK

SEQ ID NO: 865 - protein, 11 aa, Homo sapiens
RNGYRALMDKS

SEQ ID NO: 866 - protein, 10 aa, Homo sapiens
FIASNGVKLV

SEQ ID NO: 867 - protein, 9 aa, Homo sapiens
SSKALQRPV

SEQ ID NO: 868 - protein, 9 aa, Homo sapiens
FLIIWQNTM

SEQ ID NO: 869 - protein, 10 aa, Homo sapiens
ACDPHSGHFV

SEQ ID NO: 870 - protein, 9 aa, Homo sapiens
ILDKVLVHL

SEQ ID NO: 871 - protein, 9 aa, Homo sapiens
GLFGDIYLA

SEQ ID NO: 872 - protein, 9 aa, Homo sapiens
RIAECILGM

SEQ ID NO: 873 - protein, 9 aa, Homo sapiens
VVMSWAPPV

SEQ ID NO: 874 - protein, 10 aa, Homo sapiens
SLADEAEVYL

SEQ ID NO: 875 - protein, 9 aa, Homo sapiens
ILNAMIAKI

SEQ ID NO: 876 - protein, 10 aa, Homo sapiens
SLFEGIDIYT

SEQ ID NO: 877 - protein, 9 aa, Homo sapiens
FLDEFMEGV

SEQ ID NO: 878 - protein, 10 aa, Homo sapiens
SLYKFSPFPL

SEQ ID NO: 879 - protein, 9 aa, Homo sapiens
VVPCEPPEV

SEQ ID NO: 880 - protein, 10 aa, Homo sapiens
LLLDDLLVSI

SEQ ID NO: 881 - protein, 9 aa, Homo sapiens
RLSSCVPVA

SEQ ID NO: 882 - protein, 9 aa, Homo sapiens
VVPCEPPEV

SEQ ID NO: 883 - protein, 10 aa, Homo sapiens
YLCSGSSYFV

SEQ ID NO: 884 - protein, 11 aa, Homo sapiens
SLIAAAAFCLA

SEQ ID NO: 885 - protein, 9 aa, Homo sapiens
FLDRFLSCM

SEQ ID NO: 886 - protein, 10 aa, Homo sapiens
VLPDVFIRCV

SEQ ID NO: 887 - protein, 9 aa, Homo sapiens
MLAVISCAV

SEQ ID NO: 888 - protein, 11 aa, Homo sapiens
MLMAQEALAFL

SEQ ID NO: 889 - protein, 11 aa, Homo sapiens
MLMAQEALAFL

SEQ ID NO: 890 - protein, 9 aa, Homo sapiens
SLLMWITQC

SEQ ID NO: 891 - protein, 8 aa, Homo sapiens
FLGPWAAS

SEQ ID NO: 892 - protein, 9 aa, Homo sapiens
KVLEYVIKV

SEQ ID NO: 893 - protein, 9 aa, Homo sapiens
KVLEYVIKV

SEQ ID NO: 894 - protein, 9 aa, Homo sapiens
GLYDGMEHL

SEQ ID NO: 895 - protein, 9 aa, Homo sapiens
FLWGPRALV

SEQ ID NO: 896 - protein, 10 aa, Homo sapiens
YLQLVFGIEV

SEQ ID NO: 897 - protein, 9 aa, Homo sapiens
KVAELVHFL

SEQ ID NO: 898 - protein, 9 aa, Homo sapiens
FLWGPRALV

SEQ ID NO: 899 - protein, 10 aa, Homo sapiens
GVYDGREHTV

SEQ ID NO: 900 - protein, 9 aa, Homo sapiens
ALSVMGVYV

SEQ ID NO: 901 - protein, 10 aa, Homo sapiens
ILFGISLREV

SEQ ID NO: 902 - protein, 9 aa, Homo sapiens
KVVEFLAML

SEQ ID NO: 903 - protein, 9 aa, Homo sapiens
ALKDVEERV

SEQ ID NO: 904 - protein, 10 aa, Homo sapiens
LLFGLALIEV

SEQ ID NO: 905 - protein, 9 aa, Homo sapiens
SLLMWITQC

SEQ ID NO: 906 - protein, 9 aa, Homo sapiens
SLLMWITQC

SEQ ID NO: 907 - protein, 9 aa, Homo sapiens
SLLMWITQC

SEQ ID NO: 908 - protein, 11 aa, Homo sapiens
MLMAQEALAFL

SEQ ID NO: 909 - protein, 11 aa, Homo sapiens
MLMAQEALAFL

SEQ ID NO: 910 - protein, 9 aa, Homo sapiens
KASEKIFYV

SEQ ID NO: 911 - protein, 9 aa, Homo sapiens
SLGWLFLLL

SEQ ID NO: 912 - protein, 9 aa, Homo sapiens
RQKKIRIQL

SEQ ID NO: 913 - protein, 9 aa, Homo sapiens
YLSGANLNL

SEQ ID NO: 914 - protein, 9 aa, Homo sapiens
GVLVGVALI

SEQ ID NO: 915 - protein, 9 aa, Homo sapiens
IMIGVLVGV

SEQ ID NO: 916 - protein, 9 aa, Homo sapiens
KTWGQYWQV

SEQ ID NO: 917 - protein, 10 aa, Homo sapiens
AMLGTHTMEV

SEQ ID NO: 918 - protein, 9 aa, Homo sapiens
KTWGQYWQV

SEQ ID NO: 919 - protein, 9 aa, Homo sapiens
ITDQVPFSV

SEQ ID NO: 920 - protein, 9 aa, Homo sapiens
YLEPGPVTA

SEQ ID NO: 921 - protein, 10 aa, Homo sapiens
VLYRYGSFSV

SEQ ID NO: 922 - protein, 10 aa, Homo sapiens
LLDGTATLRL

SEQ ID NO: 923 - protein, 9 aa, Homo sapiens
RLMKQDFSV

SEQ ID NO: 924 - protein, 10 aa, Homo sapiens
SLADTNSLAV

SEQ ID NO: 925 - protein, 9 aa, Homo sapiens
RLPRIFCSC

SEQ ID NO: 926 - protein, 9 aa, Homo sapiens
ILTVILGVL

SEQ ID NO: 927 - protein, 10 aa, Homo sapiens
EAAGIGILTV

SEQ ID NO: 928 - protein, 9 aa, Homo sapiens
SLSKILDTV

SEQ ID NO: 929 - protein, 9 aa, Homo sapiens
TLMSAMTNL

SEQ ID NO: 930 - protein, 9 aa, Homo sapiens
ALDVYNGLL

SEQ ID NO: 931 - protein, 9 aa, Homo sapiens
FLFLLFFWL

SEQ ID NO: 932 - protein, 10 aa, Homo sapiens
FLTPKKLQCV

SEQ ID NO: 933 - protein, 10 aa, Homo sapiens
VISNDVCAQV

SEQ ID NO: 934 - protein, 9 aa, Homo sapiens
VLHWDPETV

SEQ ID NO: 935 - protein, 9 aa, Homo sapiens
SVYDFFVWL

SEQ ID NO: 936 - protein, 9 aa, Homo sapiens
TLDSQVMSL

SEQ ID NO: 937 - protein, 9 aa, Homo sapiens
MLLAVLYCL

SEQ ID NO: 938 - protein, 10 aa, Homo sapiens
CLLWSFQTSA

SEQ ID NO: 939 - protein, 9 aa, Homo sapiens
YMDGTMSQV

SEQ ID NO: 940 - protein, 9 aa, Homo sapiens
YMDGTMSQV

SEQ ID NO: 941 - protein, 9 aa, Homo sapiens
SVASTITGV

SEQ ID NO: 942 - protein, 9 aa, Homo sapiens
LLYKLADLI

SEQ ID NO: 943 - protein, 9 aa, Homo sapiens
GVALQTMKQ

SEQ ID NO: 944 - protein, 9 aa, Homo sapiens
FMNKFIYEI

SEQ ID NO: 945 - protein, 10 aa, Homo sapiens
YLNDHLEPWI

SEQ ID NO: 946 - protein, 9 aa, Homo sapiens
CQWGRLWQL

SEQ ID NO: 947 - protein, 10 aa, Homo sapiens
VLLQAGSLHA

SEQ ID NO: 948 - protein, 9 aa, Homo sapiens
FLALSILVL

SEQ ID NO: 949 - protein, 10 aa, Homo sapiens
LLAALVQDYL

SEQ ID NO: 950 - protein, 10 aa, Homo sapiens
CMLGTYTQDF

SEQ ID NO: 951 - protein, 9 aa, Homo sapiens
LLNAFTVTV

SEQ ID NO: 952 - protein, 10 aa, Homo sapiens
LMLQNALTTM

SEQ ID NO: 953 - protein, 9 aa, Homo sapiens
KVHPVIWSL

SEQ ID NO: 954 - protein, 9 aa, Homo sapiens
LLGATCMFV

SEQ ID NO: 955 - protein, 10 aa, Homo sapiens
ALGGHPLLGV

SEQ ID NO: 956 - protein, 10 aa, Homo sapiens
ALGGHPLLGV

SEQ ID NO: 957 - protein, 9 aa, Homo sapiens
TMNGSKSPV

SEQ ID NO: 958 - protein, 9 aa, Homo sapiens
FMVEDETVL

SEQ ID NO: 959 - protein, 10 aa, Homo sapiens
FINDEIFVEL

SEQ ID NO: 960 - protein, 9 aa, Homo sapiens
HLSTAFARV

SEQ ID NO: 961 - protein, 9 aa, Homo sapiens
FVGEFFTDV

SEQ ID NO: 962 - protein, 9 aa, Homo sapiens
TIHDSIQYV

SEQ ID NO: 963 - protein, 9 aa, Homo sapiens
RLAPFVYLL

SEQ ID NO: 964 - protein, 9 aa, Homo sapiens
SLLSGDWVL

SEQ ID NO: 965 - protein, 9 aa, Homo sapiens
GLQLGVQAV

SEQ ID NO: 966 - protein, 9 aa, Homo sapiens
PLTEYIQPV

SEQ ID NO: 967 - protein, 9 aa, Homo sapiens
KIFGSLAFL

SEQ ID NO: 968 - protein, 9 aa, Homo sapiens
IISAVVGIL

SEQ ID NO: 969 - protein, 9 aa, Homo sapiens
ALCRWGLLL

SEQ ID NO: 970 - protein, 9 aa, Homo sapiens
ILHNGAYSL

SEQ ID NO: 971 - protein, 9 aa, Homo sapiens
VVLGVVFGI

SEQ ID NO: 972 - protein, 10 aa, Homo sapiens
YMIMVKCWMI

SEQ ID NO: 973 - protein, 10 aa, Homo sapiens
YLVPQQGFFC

SEQ ID NO: 974 - protein, 9 aa, Homo sapiens
RLLQETELV

SEQ ID NO: 975 - protein, 9 aa, Homo sapiens
HLYQGCQVV

SEQ ID NO: 976 - protein, 9 aa, Homo sapiens
PLQPEQLQV

SEQ ID NO: 977 - protein, 9 aa, Homo sapiens
ALIHHNTHL

SEQ ID NO: 978 - protein, 9 aa, Homo sapiens
TLEEITGYL

SEQ ID NO: 979 - protein, 9 aa, Homo sapiens
PLTSIISAV

SEQ ID NO: 980 - protein, 9 aa, Homo sapiens
FLLGLIFLL

SEQ ID NO: 981 - protein, 9 aa, Homo sapiens
RLMNDMTAV

SEQ ID NO: 982 - protein, 9 aa, Homo sapiens
ALLEIASCL

SEQ ID NO: 983 - protein, 9 aa, Homo sapiens
NLSSAEVVV

SEQ ID NO: 984 - protein, 9 aa, Homo sapiens
WLPFGFILI

SEQ ID NO: 985 - protein, 9 aa, Homo sapiens
FLGYLILGV

SEQ ID NO: 986 - protein, 10 aa, Homo sapiens
AQPDTAPLPV

SEQ ID NO: 987 - protein, 9 aa, Homo sapiens
LLSDDDVVV

SEQ ID NO: 988 - protein, 9 aa, Homo sapiens
CIAEQYHTV

SEQ ID NO: 989 - protein, 10 aa, Homo sapiens
FLPEFGISSA

SEQ ID NO: 990 - protein, 8 aa, Homo sapiens
VLFYLGQY

SEQ ID NO: 991 - protein, 9 aa, Homo sapiens
TLNDECWPA

SEQ ID NO: 992 - protein, 9 aa, Homo sapiens
ALLALTSAV

SEQ ID NO: 993 - protein, 9 aa, Homo sapiens
AQCQETIRV

SEQ ID NO: 994 - protein, 10 aa, Homo sapiens
GLPPDVQRVH

SEQ ID NO: 995 - protein, 9 aa, Homo sapiens
STAPPVHNV

SEQ ID NO: 996 - protein, 9 aa, Homo sapiens
LLLLTVLTV

SEQ ID NO: 997 - protein, 9 aa, Homo sapiens
VLVPPLPSL

SEQ ID NO: 998 - protein, 9 aa, Homo sapiens
RMPEAAPPV

SEQ ID NO: 999 - protein, 9 aa, Homo sapiens
LLGRNSFEV

SEQ ID NO: 1000 - protein, 9 aa, Homo sapiens
TLPGYPPHV

SEQ ID NO: 1001 - protein, 9 aa, Homo sapiens
VLCSIDWFM

SEQ ID NO: 1002 - protein, 9 aa, Homo sapiens
VLDGLDVLL

SEQ ID NO: 1003 - protein, 10 aa, Homo sapiens
ALYVDSLFFL

SEQ ID NO: 1004 - protein, 10 aa, Homo sapiens
SLYSFPEPEA

SEQ ID NO: 1005 - protein, 9 aa, Homo sapiens
SLLQHLIGL

SEQ ID NO: 1006 - protein, 9 aa, Homo sapiens
LKLSGVVRL

SEQ ID NO: 1007 - protein, 11 aa, Homo sapiens
PLPPARNGGLG

SEQ ID NO: 1008 - protein, 9 aa, Homo sapiens
LAALPHSCL

SEQ ID NO: 1009 - protein, 10 aa, Homo sapiens
ALWPWLLMAT

SEQ ID NO: 1010 - protein, 9 aa, Homo sapiens
KMDAEHPEL

SEQ ID NO: 1011 - protein, 9 aa, Homo sapiens
AWISKPPGV

SEQ ID NO: 1012 - protein, 10 aa, Homo sapiens
SAWISKPPGV

SEQ ID NO: 1013 - protein, 9 aa, Homo sapiens
MIAVFLPIV

SEQ ID NO: 1014 - protein, 15 aa, Homo sapiens
HQQYFYKIPILVINK

SEQ ID NO: 1015 - protein, 10 aa, Homo sapiens
ELTLGEFLKL

SEQ ID NO: 1016 - protein, 10 aa, Homo sapiens
ELTLGEFLKL

SEQ ID NO: 1017 - protein, 10 aa, Homo sapiens
ILAKFLHWLE

SEQ ID NO: 1018 - protein, 9 aa, Homo sapiens
RLVDDFLLV

SEQ ID NO: 1019 - protein, 9 aa, Homo sapiens
RLARLALVL

SEQ ID NO: 1020 - protein, 9 aa, Homo sapiens
RLLVPTQFV

SEQ ID NO: 1021 - protein, 120 aa, synthetic construct
  REGION 1..120: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1022 - protein, 113 aa, synthetic construct
  REGION 1..113: note = Synthetic
DLVMTQSPSSLSASVGDRVTITCKSSQSLFNSRTRKNYLAWYQQKPGKAPKLLIYWASTRESGVPSRFSGSGSGTDFTLTISSLQPEDFATYYCQQYYYHMYTFGQGTKVEIK

SEQ ID NO: 1023 - protein, 116 aa, synthetic construct
  REGION 1..116: note = Synthetic
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 1024 - protein, 111 aa, synthetic construct
  REGION 1..111: note = Synthetic
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 1025 - protein, 107 aa, synthetic construct
  REGION 1..107: note = Synthetic
SIVMTQTPKFLPVSAGDRVTMTCKASQSVGNNVAWYQQKPGQSPKLLIYYASNRYTGVPDRFTGSGSGTDFTFTISSVQVEDLAVYFCQQHYSSPWTFGGGTKLEIK

SEQ ID NO: 1026 - protein, 116 aa, synthetic construct
  REGION 1..116: note = Synthetic
QVQLQESGPGLVKSSETLSLTCTVSGGSISSYFWSWIRQPPGKGLEWIGYIYYSGSTNYNPSLKSRVTISLHTSKNQFSLKLSSVTAADTAVYYCARHRNWLFDYWGQGTLVTVSS

SEQ ID NO: 1027 - protein, 110 aa, synthetic construct
  REGION 1..110: note = Synthetic
QSALTQPRSVSGSPGQSVTISCTGTSSDVGGYNYVSWYQQHPGKAPKVMIYDVSKRPSGVPDRFSGSKSGNTASLTISGLQAEDEADYYCCSYAGSYTYVFGTGTKVTVL

SEQ ID NO: 1028 - protein, 120 aa, synthetic construct
  REGION 1..120: note = Synthetic
EVQLVESGGGLVQPGGSLRLSCAANLVPMVATVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1029 - protein, 121 aa, synthetic construct
  REGION 1..121: note = Synthetic
EVQLVESGGGLVQPGGSLRLSCAASNLVPMVATVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1030 - protein, 122 aa, synthetic construct
  REGION 1..122: note = Synthetic
EVQLVESGGGLVQPGGSLRLSCAASNLVPMVATVVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1031 - protein, 122 aa, synthetic construct
  REGION 1..122: note = Synthetic
EVQLVESGGGLVQPGGSLRLSCAASGNLVPMVATVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1032 - protein, 123 aa, synthetic construct
  REGION 1..123: note = Synthetic
EVQLVESGGGLVQPGGSLRLSCAASGYNLVPMVATVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1033 - protein, 120 aa, synthetic construct
  REGION 1..120: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGNLVPMVATVYWGQGTTVTVSS

SEQ ID NO: 1034 - protein, 121 aa, synthetic construct
  REGION 1..121: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARNLVPMVATVMDYWGQGTTVTVSS

SEQ ID NO: 1035 - protein, 122 aa, synthetic construct
  REGION 1..122: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARNLVPMVATVAMDYWGQGTTVTVSS

SEQ ID NO: 1036 - protein, 123 aa, synthetic construct
  REGION 1..123: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGNLVPMVATVAMDYWGQGTTVTVSS

SEQ ID NO: 1037 - protein, 122 aa, synthetic construct
  REGION 1..122: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGNLVPMVATVMDYWGQGTTVTVSS

SEQ ID NO: 1038 - protein, 124 aa, synthetic construct
  REGION 1..124: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGANLVPMVATVAMDYWGQGTTVTVSS

SEQ ID NO: 1039 - protein, 123 aa, synthetic construct
  REGION 1..123: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGANLVPMVATVMDYWGQGTTVTVSS

SEQ ID NO: 1040 - protein, 125 aa, synthetic construct
  REGION 1..125: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGANLVPMVATVYAMDYWGQGTTVTVSS

SEQ ID NO: 1041 - protein, 115 aa, synthetic construct
  REGION 1..115: note = Synthetic
DLVMTQSPSSLSASVGDRVTITCKSSQSLFNSRTRKNYLAWYQQKPGKAPKLLIYWASTRESGVPSRFSGSGSGTDFTLTISSLQPEDFATYYCQQNLVPMVATVFGQGTKVEIK

SEQ ID NO: 1042 - protein, 116 aa, synthetic construct
  REGION 1..116: note = Synthetic
DLVMTQSPSSLSASVGDRVTITCKSSQSLFNSRTRKNYLAWYQQKPGKAPKLLIYWASTRESGVPSRFSGSGSGTDFTLTISSLQPEDFATYYCQQYNLVPMVATVFGQGTKVEIK

SEQ ID NO: 1043 - protein, 121 aa, synthetic construct
  REGION 1..121: note = Synthetic
EVQLVESGGGLVQPGGSLRLSCAASGYTFTSYVMHWVRQAPGKGLEWVSAINLVPMVATVYADSVKGRFTISRDNSRKTLYLQMNSLRAEDTAVYYCARGAYKRGYAMDYWGQGTTVTVSS

SEQ ID NO: 1044 - protein, 122 aa, synthetic construct
  REGION 1..122: note = Synthetic
EVQLVESGGGLVQPGGSLRLSCAASGDSVSSSISSLSWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1045 - protein, 120 aa, synthetic construct
  REGION 1..120: note = Synthetic
EVQLVESGGGLVQPGGSLRLSCAASGYTFTSYVMHWVRQAPGKGLEWVSAILPFNDGVYFADSVKGRFTISRDNSRKTLYLQMNSLRAEDTAVYYCARGAYKRGYAMDYWGQGTTVTVSS

SEQ ID NO: 1046 - protein, 113 aa, synthetic construct
  REGION 1..113: note = Synthetic
DLVMTQSPSSLSAFLGERVTLTCKSSQSLFNSRTRKNYLAWYQQKPGKAPKLLIYWASTRESGVPSRFSGSGSGTDFTLTISSLQPEDFATYYCQQYYYHMYTFGQGTKVEIK

SEQ ID NO: 1047 - protein, 120 aa, synthetic construct
  REGION 1..120: note = Synthetic
EVQLVESGGGLVQPGGSLRLSCLGGLLTMVSYVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1048 - protein, 121 aa, synthetic construct
  REGION 1..121: note = Synthetic
EVQLVESGGGLVQPGGSLRLSCLGGLLTMVTSYVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1049 - protein, 121 aa, synthetic construct
  REGION 1..121: note = Synthetic
EVQLVESGGGLVQPGGSLRLSCLGGLLTMVSSYVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1050 - protein, 123 aa, synthetic construct
  REGION 1..123: note = Synthetic
EVQLVESGGGLVQPGGSLRLSCAASGILGFVFTLYVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1051 - protein, 120 aa, synthetic construct
  REGION 1..120: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGILGFVFTLDYWGQGTTVTVSS

SEQ ID NO: 1052 - protein, 121 aa, synthetic construct
  REGION 1..121: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGILGFVFTLMDYWGQGTTVTVSS

SEQ ID NO: 1053 - protein, 122 aa, synthetic construct
  REGION 1..122: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGILGFVFTLAMDYWGQGTTVTVSS

SEQ ID NO: 1054 - protein, 120 aa, synthetic construct
  REGION 1..120: note = Synthetic
EVQLVESGGGLVQPGGSLRLSCAVFDRKSDAKVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1055 - protein, 121 aa, synthetic construct
  REGION 1..121: note = Synthetic
EVQLVESGGGLVQPGGSLRLSCAVFDRKSDAKYVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1056 - protein, 113 aa, synthetic construct
  REGION 1..113: note = Synthetic
DLVMTQSPSSLSASVGDRVTITCKSSQSLFNSRTRKNYLAWYQQKPGKAPKLLIYWASTRESGVPSRFSGSGSGTDFTLTISSLQPEDFATYYCQQYDPVAALFGQGTKVEIK

SEQ ID NO: 1057 - protein, 115 aa, synthetic construct
  REGION 1..115: note = Synthetic
DLVMTQSPSSLSASVGDRVTITCKSSQSLFNSRTRKNYLAWYQQKPGKAPKLLIYWASTRESGVPSRFSGSGSGTDFTLTISSLQPEDFATYYCQQYQYDPVAALFGQGTKVEIK

SEQ ID NO: 1058 - protein, 122 aa, synthetic construct
  REGION 1..122: note = Synthetic
EVQLVESGGGLVQPGGSLRLSCAASQYDPVAALFVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 1059 - protein, 123 aa, synthetic construct
  REGION 1..123: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGQYDPVAALFAMDYWGQGTTVTVSS

SEQ ID NO: 1060 - protein, 111 aa, synthetic construct
  REGION 1..111: note = Synthetic
DIVLTQSPASLAVSLGQRATISCRASKSVSSSISSLMHWYQQKPGQPPKLLIKYASYLESGVPARFSGSGSGTDFTLNIHPVEEEDAATYYCQHSREFPWTFGGGTKLEIK

SEQ ID NO: 1061 - protein, 111 aa, synthetic construct
  REGION 1..111: note = Synthetic
DIVLTQSPASLAVSLGQRATISCRASKSVSTSSYSYMHWYQQKPGQPPKLLALHRSYLESGVPARFSGSGSGTDFTLNIHPVEEEDAATYYCQHSREFPWTFGGGTKLEIK

SEQ ID NO: 1062 - protein, 111 aa, synthetic construct
  REGION 1..111: note = Synthetic
DIVLTQSPASLAVSLGQRATISCRASNLVPMVATVYMHWYQQKPGQPPKLLIKYASYLESGVPARFSGSGSGTDFTLNIHPVEEEDAATYYCQHSREFPWTFGGGTKLEIK

SEQ ID NO: 1063 - protein, 111 aa, synthetic construct
  REGION 1..111: note = Synthetic
DIVLTQSPASLAVSLGQRATISCRASKNLVPMVATVMHWYQQKPGQPPKLLIKYASYLESGVPARFSGSGSGTDFTLNIHPVEEEDAATYYCQHSREFPWTFGGGTKLEIK

SEQ ID NO: 1064 - protein, 111 aa, synthetic construct
  REGION 1..111: note = Synthetic
DIVLTQSPASLAVSLGQRATISCRASKGILGFVFTLMHWYQQKPGQPPKLLIKYASYLESGVPARFSGSGSGTDFTLNIHPVEEEDAATYYCQHSREFPWTFGGGTKLEIK

SEQ ID NO: 1065 - protein, 111 aa, synthetic construct
  REGION 1..111: note = Synthetic
DIVLTQSPASLAVSLGQRATISCRASKSVSTSSYSYMHWYQQKPGQPPKNLVPMVATVESGVPARFSGSGSGTDFTLNIHPVEEEDAATYYCQHSREFPWTFGGGTKLEIK

SEQ ID NO: 1066 - protein, 110 aa, synthetic construct
  REGION 1..110: note = Synthetic
QSALTQPRSVSGSPGQSVTISCTGTSTDVASLNYVSWYQQHPGKAPKVMIYDVSKRPSGVPDRFSGSKSGNTASLTISGLQAEDEADYYCCSYAGSYTYVFGTGTKVTVL

SEQ ID NO: 1067 - protein, 116 aa, synthetic construct
  REGION 1..116: note = Synthetic
QVQLQESGPGLVKSSETLGIVCPISGGSISSYFWSWIRQPPGKGLEWIGYIYYSGSTNYNPSLKSRVTISLHTSKNQFSLKLSSVTAADTAVYYCARHRNWLFDYWGQGTLVTVSS

SEQ ID NO: 1068 - protein, 107 aa, synthetic construct
  REGION 1..107: note = Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 1069 - protein, 119 aa, synthetic construct
  REGION 1..119: note = Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 1070 - protein, 107 aa, synthetic construct [sequence omitted]

SEQ ID NO: 1071 - protein, 119 aa, synthetic construct [sequence omitted]

SEQ ID NO: 1072 - protein, 107 aa, synthetic construct [sequence omitted]

SEQ ID NO: 1073 - protein, 121 aa, synthetic construct [sequence omitted]

SEQ ID NO: 1074 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1075 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 1076 - protein, 107 aa, synthetic construct [sequence omitted]

SEQ ID NO: 1077 - protein, 218 aa, synthetic construct
  REGION 1..218: note = Synthetic
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1078 - protein, 446 aa, synthetic construct
  REGION 1..446: note = Synthetic
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 1079 - protein, 214 aa, synthetic construct
  REGION 1..214: note = Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1080 - protein, 449 aa, synthetic construct
  REGION 1..449: note = Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1081 - protein, 214 aa, synthetic construct
  REGION 1..214: note = Synthetic
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1082 - protein, 449 aa, synthetic construct
  REGION 1..449: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1083 - protein, 220 aa, synthetic construct
  REGION 1..220: note = Synthetic
DLVMTQSPSSLSASVGDRVTITCKSSQSLFNSRTRKNYLAWYQQKPGKAPKLLIYWASTRESGVPSRFSGSGSGTDFTLTISSLQPEDFATYYCQQYYYHMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1084 - protein, 450 aa, synthetic construct
  REGION 1..450: note = Synthetic
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGAYKRGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 1085 - protein, 214 aa, synthetic construct
  REGION 1..214: note = Synthetic
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1086 - protein, 451 aa, synthetic construct
  REGION 1..451: note = Synthetic
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1087 - protein, 218 aa, synthetic construct
  REGION 1..218: note = Synthetic
DIVLTQSPASLAVSLGQRATISCRASKNLVPMVATVMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1088 - protein, 446 aa, synthetic construct; identical to SEQ ID NO: 1078
  REGION 1..446: note = Synthetic

SEQ ID NO: 1089 - protein, 220 aa, synthetic construct; identical to SEQ ID NO: 1083
  REGION 1..220: note = Synthetic

SEQ ID NO: 1090 - protein, 451 aa, synthetic construct
  REGION 1..451: note = Synthetic
EVQLVESGGGLVQPGGSLRLSCAASGYTFTSYVMHWVRQAPGKGLEWVSAINLVPMVATVYADSVKGRFTISRDNSRKTLYLQMNSLRAEDTAVYYCARGAYKRGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

(19) United States
(12) Patent Application Publication
(10) Pub. No.: US 2025/0255955 A1
(43) Pub. Date: Aug. 14, 2025

(54) DUAL FUNCTION ANTIGEN BINDING MOLECULES

(71) Applicant: TROJAN BIO LTD., Ganei Tikva (IL)

(72) Inventors: Anat BURKOVITZ, Ganei Tikva (IL); Noam C. DIAMANT, Ein Vered (IL); Amir AHARONI, Beit Kama (IL)

(21) Appl. No.: 18/702,391

(22) PCT Filed: Oct. 18, 2022
(86) PCT No.: PCT/IL2022/051102

§ 371 Date: Apr. 18, 2024

(30) Foreign Application Priority Data
Oct. 18, 2021 (IL) 287372

(51) Int. Cl. A61K 39/385; A61K 39/00; A61K 39/12; A61P 35/00; C07K 14/005; C07K 16/28; C07K 16/46; C12N 7/00

(52) U.S. Cl. CPC A61K 39/385; A61K 39/12; A61P 35/00; C07K 14/005; C07K 16/2863; C07K 16/46; C12N 7/00; A61K 2039/6056; C07K 2317/565; C12N 2710/16122; C12N 2710/16134; C12N 2710/16222; C12N 2710/16234; C12N 2760/16121; C12N 2760/16134; C12N 2770/20011; C12N 2770/20034

(57) ABSTRACT

Antibodies or antigen binding fragments thereof comprising at least one immunogenic peptide inserted into a variable region of the antibody or antigen binding fragment thereof, wherein the insertion comprises removal of antibody or antigen binding fragment sequence are provided. Dual-function antigen binding molecules comprising an antibody or antigen binding fragment of the invention and a second antibody or antigen binding fragment thereof capable of binding an antigen overexpressed on a target cell are provided. Nucleic acid molecules encoding same, pharmaceutical compositions comprising same and methods of treating cancer by administrating same are also provided. Methods of producing antibodies or antigen binding fragments are also provided.

### CROSS REFERENCE TO RELATED APPLICATIONS

This application claims the benefit of priority of Israeli Patent Application No. 287372 filed on Oct. 18, 2021, the contents of which are incorporated herein by reference in its entirety.

### FIELD OF INVENTION

The present invention is in the field of anti-cancer immunotherapy.

### SUMMARY OF THE INVENTION

The present invention provides antibodies or antigen binding fragments thereof comprising at least one immunogenic peptide inserted into a variable region of the antibody or antigen binding fragment thereof. Dual-function antigen binding molecules comprising an antibody or antigen binding fragment of the invention and a second antibody or antigen binding fragment thereof capable of binding an antigen overexpressed on a target cell. Nucleic acid molecules encoding same, pharmaceutical compositions comprising same and methods of treating cancer by administrating same are also provided. Methods of producing antibodies or antigen binding fragments are also provided.

According to a first aspect, there is provided an antibody or antigen binding fragment thereof comprising at least one immunogenic peptide inserted into a variable region of the antibody or antigen binding fragment thereof, and where the insertion comprises removal of antibody or antigen binding fragment sequence.

According to another aspect, there is provided a dual-function antigen binding molecule comprising:
a. a first antibody or antigen binding fragment thereof comprising at least one immunogenic peptide inserted into a CDR of the antibody or antigen binding fragment thereof, and where the insertion comprises removal of CDR sequence; and
b. a second antibody capable of binding epidermal growth factor receptor (EGFR), wherein the antibody is selected from cetuximab, panitumumab and necitumumab or antibody comprising at least 85% sequence identity thereto.

According to some embodiments, the antibody or antigen binding fragment thereof binds to a target cell, and wherein the target cell is a cancer cell, a dendritic cell or both. According to some embodiments, the first antibody or antigen binding fragment thereof binds to a target cell, and wherein the target cell is a cancer cell, a dendritic cell or both.

According to some embodiments, the immunogenic peptide is a cancer specific peptide.

According to some embodiments, the cancer specific peptide is selected from a peptide sequence provided in Table 1.

According to some embodiments, the immunogenic peptide is a viral peptide.

According to some embodiments, the viral peptide is derived from Cytomegalovirus (CMV), Epstein-Barr virus (EBV), Severe acute respiratory syndrome coronavirus 2 (SARS-CoV2), Adenovirus, Human papilloma virus (HPV) or Influenza virus (FLU).

According to some embodiments, the viral peptide is selected from a peptide sequence provided in Table 2 or Table 3.

According to some embodiments, the antibody or antigen binding fragment thereof further comprises a cell penetration sequence that targets the antibody or antigen binding fragment thereof to a cytoplasm of a cell binding the antibody or antigen binding fragment thereof. According to some embodiments, the antibody or antigen binding fragment thereof further comprises a cell penetration sequence that targets the first antibody or antigen binding fragment thereof to a cytoplasm of a cell bound by the first antibody or antigen binding fragment thereof.

According to some embodiments, the cell penetration sequence is an endosomal escape domain (EED).

According to some embodiments, the antibody devoid of the immunogenic peptide is endocytosed into an endosomal pathway and is delivered to the cytoplasm. According to some embodiments, the first antibody or antigen binding fragment thereof is endocytosed into an endosomal pathway and is delivered to the cytoplasm.

According to some embodiments, at least one CDR or a portion thereof of the antibody or antigen binding fragment thereof is replaced with the immunogenic peptide. According to some embodiments, at least one CDR or a portion thereof of the first antibody or antigen binding fragment thereof is replaced with the immunogenic peptide.

According to some embodiments, the CDR is an inert CDR having little or no contribution to binding to a target antigen.

According to some embodiments, an inert CDR comprises two or fewer amino acids that contact the target antigen.

According to some embodiments, contact comprises a distance of not more than 5 angstroms between an amino acid of a CDR and an amino acid of the target antigen.

According to some embodiments, the insertion and removal produces no change or minimal change in the overall conformation of the antibody or antigen binding fragment thereof such that the antibody or antigen binding fragment thereof binds its target antigen at an equivalent affinity to the antibody or antigen binding fragment devoid of the immunogenic peptide. According to some embodiments, the insertion and removal produces no change or minimal change in the overall conformation of the first antibody or antigen binding fragment thereof such that the first antibody or antigen binding fragment thereof binds its target antigen at an equivalent affinity to the first antibody or antigen binding fragment devoid of the immunogenic peptide.

According to some embodiments, at least one inert CDR of an antigen binding region is replaced with a cell penetration sequence.

According to some embodiments, the target cell is a dendritic cell and a dendritic cell antigen is selected from CD40, CD205, CD206, CLEC9A, CLEC12A, CD209, and CD207.

According to some embodiments, the target cell is a malignant immune cell and an immune cell antigen is selected from CD20, CD19, CD21, and CD22.

According to some embodiments, the target cell is a cancer cell and a cancer cell antigen is selected from HER2, EGFR, EpCAM, PSMA, BCMA, CD123, CD33, CD38, CTLA, LAG-3, ICOS, 4-1BB and PD-L1.

According to some embodiments, the antibody or antigen binding fragment thereof is devoid of a chemical linker.

According to some embodiments, the antigen binding region, the immunogenic peptide and the cell penetrating sequence are each separated by a linker. According to some embodiments, within the first antibody or antigen binding fragment thereof the antigen binding region, the immunogenic peptide and the cell penetrating sequence are each separated by a linker.

According to some embodiments, the immunogenic peptide is recognized by CD4 T cells, CD8 T cells or both.

According to some embodiments, the antibody before the immunogenic peptide is inserted is selected from:
a. antibody TMab4 comprising a heavy chain variable region of SEQ ID NO: 1021 and a light chain variable region of SEQ ID NO: 1022;
b. antibody 3E10 comprising a heavy chain variable region of SEQ ID NO: 1023 and a light chain variable region of SEQ ID NO: 1024; and
c. antibody 71F12 comprising a heavy chain variable region of SEQ ID NO: 1026 and a light chain variable region of SEQ ID NO: 1027.

According to some embodiments, the first antibody before the immunogenic peptide is inserted is selected from:
a. antibody TMab4 comprising a heavy chain variable region of SEQ ID NO: 1021 and a light chain variable region of SEQ ID NO: 1022;
b. antibody 3E10 comprising a heavy chain variable region of SEQ ID NO: 1023 and a light chain variable region of SEQ ID NO: 1024; and
c. antibody 71F12 comprising a heavy chain variable region of SEQ ID NO: 1026 and a light chain variable region of SEQ ID NO: 1027.

According to some embodiments, at least one of:
a. the immunogenic peptide is inserted into CDRH1, CDRH2, CDRH3 or cDRL3 of the TMab4;
b. the immunogenic peptide is inserted into CDRL1 or CDRL2 of the 3E10; and
c. the immunogenic peptide is inserted into CDRL1 of the 71F12.

According to some embodiments, the antibody or antigen binding fragment thereof comprises at least one of:
a. a light chain variable region of SEQ ID NO: 1022 and a heavy chain variable region selected from SEQ ID NO: 1028-1040, 1043-1045, 1047-1055, and 1058-1059;
b. a heavy chain variable region of SEQ ID NO: 1021 and a light chain variable region selected from SEQ ID NO: 1041-1042, 1046, and 1056-1057;
c. a heavy chain variable region of SEQ ID NO: 1023 and a light chain variable region selected from SEQ ID NO: 1060-1065;
d. a heavy chain variable region of SEQ ID NO: 1026 and a light chain variable region of SEQ ID NO: 1066; and
e. a light chain variable region of SEQ ID NO: 1027 and a heavy chain variable region of SEQ ID NO: 1067.

According to some embodiments, the first antibody comprises at least one of:
a. a light chain variable region of SEQ ID NO: 1022 and a heavy chain variable region selected from SEQ ID NO: 1028-1040, 1043-1045, 1047-1055, and 1058-1059;
b. a heavy chain variable region of SEQ ID NO: 1021 and a light chain variable region selected from SEQ ID NO: 1041-1042, 1046, and 1056-1057;
c. a heavy chain variable region of SEQ ID NO: 1023 and a light chain variable region selected from SEQ ID NO: 1060-1065;
d. a heavy chain variable region of SEQ ID NO: 1026 and a light chain variable region of SEQ ID NO: 1066; and
e. a light chain variable region of SEQ ID NO: 1027 and a heavy chain variable region of SEQ ID NO: 1067.

According to another aspect, there is provided a dual-function antigen binding molecule comprising a first antibody or antigen binding fragment thereof comprising an antibody or antigen binding fragment of the invention and a second antibody or antigen binding fragment thereof capable of binding an antigen overexpressed on a target cancer cell.

According to some embodiments, the antigen overexpressed on a target cancer cell is EGFR and the second antibody is selected from cetuximab, panitumumab and necitumumab.

According to some embodiments, the first antibody and the second antibody comprise at least one modification that promotes heterodimerization and inhibit homodimerization.

According to some embodiments, one of the first and second antibody comprises a heavy chain constant region comprising SEQ ID NO: 1074 and the other antibody comprises a heavy chain constant region comprising SEQ ID NO: 1075.

According to some embodiments, the dual function antigen binding molecule comprises two heavy chains and two light chains, wherein:
a. the two heavy chains are SEQ ID NO: 1088 and 1080 and the two light chains are SEQ ID NO: 1087 and 1079;
b. the two heavy chains are SEQ ID NO: 1088 and 1082 and the two light chains are SEQ ID NO: 1087 and 1081;
c. the two heavy chains are SEQ ID NO: 1090 and 1080 and the two light chains are SEQ ID NO: 1089 and 1079;
d. the two heavy chains are SEQ ID NO: 1090 and 1082 and the two light chains are SEQ ID NO: 1089 and 1081; or
e. the two heavy chains are SEQ ID NO: 1088 and 1086 and the two light chains are SEQ ID NO: 1087 and 1085.

According to another aspect, there is provided a pharmaceutical composition comprising an antibody or antigen binding fragment or the invention or a dual-function antigen binding molecule of the invention and a pharmaceutically acceptable carrier excipient or adjuvant.

According to another aspect, there is provided a nucleic acid molecule comprising at least one open reading frame, wherein the open reading frame encodes an antibody or antigen binding fragment thereof of the invention or a dual-function antigen binding molecule the invention.

According to another aspect, there is provided an expression vector comprising at least one regulatory element operatively linked to a nucleic acid molecule of the invention.

According to another aspect, there is provided a method of treating cancer in a subject in need thereof, the method comprising administering to the subject a pharmaceutical composition of the invention, thereby treating cancer in a subject.

According to some embodiments, the cancer overexpresses the cancer specific antigen.

According to some embodiments, the cancer is an EGFR positive cancer.

According to some embodiments, the dual-function antigen binding molecule is a cancer vaccine and comprises an antigen binding region capable of binding a dendritic cell antigen.

According to another aspect, there is provided a method of engineering an antibody or antigen binding fragment thereof, the method comprising:
a. selecting an antibody or antigen binding fragment thereof of interest;
b. receiving structural analysis of the selected antibody or antigen binding domain bound to its target;
c. determining at least one CDR of the selected antibody or antigen binding domain that is not required for binding to the target based on the structural analysis;
d. replacing the determined at least one CDR or a portion thereof with an immunogenic peptide;
   thereby engineering an antibody or antigen binding fragment thereof.

According to another aspect, there is provided a method of engineering an antibody or antigen binding fragment thereof, the method comprising:
a. selecting an antibody or antigen binding fragment thereof of interest;
b. receiving a database of immunogenic peptides;
c. performing pairwise alignment of peptides of a variable region of the selected antibody or antigen binding fragment thereof of interest with immunogenic peptides of the database;
d. determining a peptide from the selected antibody or antigen binding fragment thereof and an immunogenic peptide with an alignment score above a predetermined threshold; and
e. replacing the determined peptide from the selected antibody or antigen binding fragment thereof with the determined immunogenic peptide;
   thereby engineering an antibody or antigen binding fragment thereof.

According to some embodiments, the method further comprises optimizing the replacing to produce as little perturbation in the structure of the selected antibody or antigen binding fragment thereof of interest as possible.

According to some embodiments, the engineered antibody or antigen binding fragment thereof is an immunogenic peptide delivery antibody.

According to some embodiments, step (a) comprises selecting an antibody or antigen binding fragment thereof that binds to a surface of a target cell.

According to some embodiments, step (a) comprises selecting an antibody or antigen binding fragment thereof that upon binding to a surface is internalized and delivered to a cytosol of the target cell.

According to some embodiments, the method further comprises confirming at least one of: delivery of the immunogenic peptide to a cytosol of the target cell, delivery of the immunogenic peptide in complex with an HLA molecule to a surface of the target cell and specific killing of the target cell by an effector cell specific to the immunogenic peptide.

According to some embodiments, the method further comprises selecting a targeting antibody that binds to a protein on a surface of a target cell and producing a dual-function antigen binding molecule by combining the engineered antibody and the targeting antibody.

According to some embodiments, the combining comprises engineering a heavy chain constant region of the targeting antibody and a heavy chain constant region of the engineered antibody to promote heterodimerization and inhibit homodimerization.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: A schematic showing an antibody engaging its antigen and the presence of inert CDRs not involved in binding.

FIG. 2: Images of an anti-PD-L1 antibody with an inert CDR and the Trojan antibody of the invention after the inert CDR is replace with an immunogenic peptide.

FIG. 3: A stepwise diagram of dendritic cell vaccination method of the invention.

FIG. 4: A stepwise diagram of a B cell targeting CD4+ T cell-mediated method of the invention.

FIG. 5: A stepwise diagram of a CD8+ T cell-mediated cancer killing method of the invention.

FIG. 6: An overview of embodiments of the Trojan antibody production method.

FIGS. 7A-7C: Bar charts of specific killing of cancer cells contacted with (7A) TAbs containing immunogenic peptides inserted into CDRH1 of TMab4 (P1) antibody, (7B) TAbs containing immunogenic peptides inserted into CDRH3 of TMab4 (P1) antibody, and (7C) TAbs containing immunogenic peptides inserted into CDRL3 pr CDRH2 of TMab4 (P1) antibody, and then cocultured with peptide specific effector cells.

FIG. 8: Bar charts of specific killing of cancer cells contacted with TAbs containing immunogenic peptides inserted into CDRL1 of 3E10 (P2) antibody and then cocultured with peptide specific effector cells.

FIGS. 9A-9B: Line graphs of apoptotic cancer cells cultured with effector cells alone (Cells only), (9A) parental antibody 3E10 (P2) and TAb T2_11, and (9B) parental antibody TMab4 (P1) and TAb T18.

FIG. 10: Schematics of bi-functional Trojan antibodies and corresponding controls. Bi-TAbs are labeled as FTAbs and control bi-TAbs are labeled as cFTAbs.

FIG. 11: Histograms showing binding of bi-TAbs to EGFR on the surface of cancer cells. Therapeutic antibodies are used as the positive control and fluorescently labeled secondary antibodies are used as a negative control. Upper panels show 0.3 nM concentration and lower panels show 3 nM concentration. Bi-TAbs are labeled as FTAbs.

FIG. 12: Bar graphs of the percentage of cancer cells displaying the HLA-peptide complex at different time points. Each bi-TAb (white bars) is compared to its negative control (grey bars) with the same killing module but without the immunogenic peptide. Bi-TAbs are labeled as FTAbs and control bi-TAbs are labeled as cFTAbs.

FIG. 13: Line graphs of percentage of cancer cells killed by effector cells at various effector to target cell ratios. Each bi-TAb (light grey) is compared to its negative control (black lines). An antibody only point without effector cells is included. Bi-TAbs are labeled as FTAbs and control bi-TAbs are labeled as cFTAbs.

### DETAILED DESCRIPTION OF THE INVENTION

The present invention, in some embodiments, provides antibodies or antigen binding fragments thereof comprising at least one immunogenic peptide inserted into a variable region of the antibody or antigen binding fragment thereof, wherein the insertion comprises removal of antibody or antigen binding fragment sequence. Dual-function antigen binding molecules comprising an antibody or antigen binding fragment of the invention and a second antibody or antigen binding fragment thereof capable of binding an antigen overexpressed on a target cell. Nucleic acid molecules encoding same, pharmaceutical compositions comprising same and methods of treating cancer by administrating same are also provided. Methods of producing antibodies or antigen binding fragments are also provided.

The invention is based on the surprising finding that antibodies/antigen binding molecules can be used as a delivery system for immunogenic peptides. That is, a highly immunogenic peptide can be delivered specifically to cancer cells and thereby increase immune surveillance against them. The antibody would in this case have an antigen binding domain to a cancer epitope which would cause the therapeutic molecule to bind the cancer cell. Upon endocytosis of the antibody/antigen binding molecule the immunogenic peptide would be delivered to the cytoplasm. This can be enhanced by the inclusion of a cell penetrating sequence, or specifically an endosomal escape sequence. Endosomal escape however is not essential as some mechanisms, such as receptor-mediated transcytosis of antibodies, deliver the antibody directly to the cytoplasm. The therapeutic molecule would be cleaved, releasing the immunogenic peptide which would then be displayed on the cell surface in complex with an HLA molecule, thus enhancing the immunogenicity of the cancer cell and increasing immune surveillance against the cancer and cancer killing.

Alternatively, the antigen binding region can bind a dendritic cell antigen, which would deliver the therapeutic molecule comprising a cancer cell antigen to a dendritic cell. The molecule would be endocytosed upon binding and the immunogenic cancer peptide will be cleaved from the rest of the molecule of the invention and displayed on the surface of the dendritic cell by HLA molecules. This will in turn train cytotoxic immune cells (T cell and NK cells) to target this immunogenic peptide and thereby the cancer.

By a first aspect, there is provided an antigen binding molecule.

By another aspect, there is provided a composition comprising the antigen binding molecule of the invention.

By another aspect, there is provided a nucleic acid molecule encoding the antigen binding molecule of the invention.

By another aspect, there is provided an expression vector comprising the nucleic acid molecule of the invention.

By another aspect, there is provided a method of expressing a peptide on a surface of a target cell, the method comprising contacting the target cell with an antigen binding molecule of the invention or a pharmaceutical composition of the invention, thereby expressing a peptide on a surface of a target cell.

By another aspect, there is provided a method of treating cancer in a subject in thereof, the method comprising administering to the subject an antigen binding molecule of the invention or a pharmaceutical composition of the invention, thereby treating cancer in a subject in need thereof.

By another aspect, there is provided a composition of the invention for use in expressing a peptide on a surface of a target cell. By another aspect, there is provided a composition of the invention for use in treating cancer. By another aspect, there is provided a composition of the invention for use in the production of a medicament for the treating of cancer.

In some embodiments, the antigen binding molecule is a dual-function molecule. In some embodiments, the first function is binding an antigen. In some embodiments, the second function is entering a cell. In some embodiments, the second function is delivering a peptide. In some embodiments, the peptide is an immunogenic peptide. In some embodiments, the second function is delivering an immunogenic peptide into a cytoplasm of a cell.

In some embodiments, the molecule comprises an antigen binding region. In some embodiments, the region is a domain. In some embodiments, the region binds the antigen. In some embodiments, the antigen is on a target cell. In some embodiments, the antigen binding region is capable of binding to a target cell. In some embodiments, the antigen is a cancer antigen. In some embodiments, a cancer antigen is a cancer specific antigen. In some embodiments, a cancer antigen is an antigen on a cancer cell. In some embodiments, the antigen is an immune cell antigen. In some embodiments, the immune cell is selected from a dendritic cell, a B cell, a T cell, a neutrophil, a macrophage and a natural killer (NK) cell. In some embodiments, the immune cell is a dendritic cell. In some embodiments, the antigen is a dendritic cell antigen. In some embodiments, the immune cell is a B cell. In some embodiments, the antigen is a B cell antigen. In some embodiments, an antigen is expressed on a cell. In some embodiments, an antigen is expressed on a cell surface. In some embodiments, an antigen is displayed on the cell surface as an MHC molecule. In some embodiments, an MHC molecule is an MHC class I or class II molecule. In some embodiments, an MHC molecule is a protein complex of the antigen and an HLA protein. In some embodiments, an antigen is a cell surface protein. In some embodiments, a cell surface protein is a cell surface receptor. In some embodiments, the antigen binding region is capable of binding the antigen.

In some embodiments, a dendritic cell antigen is selected from CD40, CD205, CD206, CLEC9A, CLEC12A, CD209, and CD207. Markers of dendritic cells are well known in the art and any such surface marker may be used as the antigen. In some embodiments, the dendritic cell antigen is CD40. Antigen binding domains that target dendritic cell antigens are well known in the art and any such antigen binding domain may be employed. For a non-limiting example Fab516 binds specifically to CD40.

In some embodiments, an immune cell antigen is selected from CD20, CD19, CD21, and CD22. In some embodiments, the immune cell antigen is CD20. In some embodiments, the immune cell antigen is a B cell antigen. Markers of immune cells in general, and B cells in particular, are well known in the art and any such surface marker may be used as the antigen. Antigen binding domains that target immune cell antigens are well known in the art and any such antigen binding domain may be employed. For a non-limiting example Arzerra binds specifically to CD20

In some embodiments, a cancer cell antigen is selected from HER2, EGFR, EpCAM, PSMA, BCMA, CD123, CD33, CD38, CTLA, LAG-3, ICOS, 4-1BB and PD-L1. Markers of cancer cells are well known in the art and any such surface marker may be used as the antigen. In some embodiments, the cancer cell antigen is PD-L1. Antigen binding domains that target cancer cell antigens are well known in the art and any such antigen binding domain may be employed. For a non-limiting example Durvalumab binds specifically to PD-L1.

In some embodiments, the antigen binding domain is an antigen binding domain of an antibody. In some embodiments, the antigen binding domain is an antibody. In some embodiments, the antigen binding domain is an antibody or antigen binding fragment thereof. In some embodiments, the antibody is a single-chain antibody. In some embodiments, the antibody is a single domain antibody. In some embodiments, the antibody is a full antibody.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜ Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

An “antigen” is a molecule or a portion of a molecule capable of eliciting antibody formation and being bound by an antibody. Antibody formation can occur in mice, rats, rabbits, pigs and other animals commonly used for generation of antibodies, but of course can also occur in humans as a response to a foreign antigen. An antigen may have one or more than one epitope. The specific reaction referred to above is meant to indicate that the antigen will react, in a highly selective manner, with its corresponding antibody and not with the multitude of other antibodies which may be evoked by other antigens.

The term “antigenic determinant” or “epitope” according to the invention refers to the region of an antigen molecule that specifically reacts with particular antibody. Peptide sequences derived from an epitope can be used, alone or in conjunction with a carrier moiety, applying methods known in the art, to immunize animals and to produce additional polyclonal or monoclonal antibodies. Immunoglobulin variable domains can also be analyzed using the IMGT information system (imgt. Cines.fr/) (IMGT®/V-Quest) to identify variable region segments, including CDRs. See, e.g., Brochet, X. et al, Nucl. Acids Res. J6:W503-508 (2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al, U.S. Dept. of Health and Human Services, “Sequence of Proteins of Immunological Interest” (1983).

The term “antibody” (also referred to as an “immunoglobulin”) is used in the broadest sense and specifically encompasses monoclonal antibodies and antibody fragments so long as they exhibit the desired biological activity. In certain embodiments, the use of a chimeric antibody or a humanized antibody is also encompassed by the invention.

The basic unit of the naturally occurring antibody structure is a heterotetrameric glycoprotein complex of about 150,000 Daltons, composed of two identical light (L) chains and two identical heavy (H) chains, linked together by both noncovalent associations and by disulfide bonds. Each heavy and light chain also has regularly spaced intra-chain disulfide bridges. Five human antibody classes (IgG, IgA, IgM, IgD and IgE) exist, and within these classes, various subclasses, are recognized based on structural differences, such as the number of immunoglobulin units in a single antibody molecule, the disulfide bridge structure of the individual units, and differences in chain length and sequence. The class and subclass of an antibody is its isotype.

The amino terminal regions of the heavy and light chains are more diverse in sequence than the carboxy terminal regions, and hence are termed the variable domains. This part of the antibody structure confers the antigen-binding specificity of the antibody. A heavy variable (VH) domain and a light variable (VL) domain together form a single antigen-binding site, thus, the basic immunoglobulin unit has two antigen-binding sites. Particular amino acid residues are believed to form an interface between the light and heavy chain variable domains (Chothia et al., J. Mol. Biol. 186, 651-63 (1985); Novotny and Haber, (1985) Proc. Natl. Acad. Sci. USA 82 4592-4596).

The carboxy terminal portion of the heavy and light chains form the constant domains i.e., CH1, CH2, CH3, CL. While there is much less diversity in these domains, there are differences from one animal species to another, and further, within the same individual there are several different isotypes of antibody, each having a different function.

The term “framework region” or “FR” refers to the amino acid residues in the variable domain of an antibody, which are other than the hypervariable region amino acid residues as herein defined. The term “hypervariable region” as used herein refers to the amino acid residues in the variable domain of an antibody, which are responsible for antigen binding. The hypervariable region comprises amino acid residues from a “complementarity determining region” or “CDR”. The CDRs are primarily responsible for binding to an epitope of an antigen. The extent of FRs and CDRs has been precisely defined (see, Kabat et al.). In some embodiments, CDRs are determined using the KABAT system. In some embodiments, CDRs are determined using the Clothia system. In some embodiments, the Clothia system is the enhanced Clothia system (Martin system).

In some embodiments, the antibody or antigen binding fragment thereof is the antibody or antigen binding fragment thereof devoid or without the immunogenic peptide. In some embodiments, devoid of without the immunogenic peptide is before insertion of the immunogenic peptide. In some embodiments, the antibody or antigen binding fragment thereof binds to a target cell. In some embodiments, the antibody or antigen binding fragment thereof adheres to a surface of a target cell. In some embodiments, the antibody or antigen binding fragment thereof is a cell penetrating antibody. In some embodiments, the antibody or antigen binding fragment thereof is phagocytosed into the cell. In some embodiments, the antibody or antigen binding fragment thereof is endocytosed into the cell. In some embodiments, into the cell is into the endosomal pathway of the cell. In some embodiments, the antibody or antigen binding fragment thereof is brought into the endosomal pathway. In some embodiments, the antibody or antigen binding fragment thereof escapes from the endosomal pathway. In some embodiments, the antibody or antigen binding fragment thereof is delivered into the cytoplasm of a cell that binds it. In some embodiments, the antibody or antigen binding fragment thereof is a DNA binding antibody. In some embodiments, the antibody or antigen binding fragment thereof is a lupus antibody.

In some embodiments, the antibody or antigen binding fragment thereof is the Tmab4 antibody. In some embodiments, the Tmab4 comprises a heavy chain variable region of SEQ ID NO: 1021 or an analog or homolog comprising at least 85% sequence identity and being capable of binding cells and reaching the cytosol. In some embodiments, the Tmab4 comprises a light chain variable region of SEQ ID NO: 1022 or an analog or homolog comprising at least 85% sequence identity and being capable of binding cells and reaching the cytosol. In some embodiments, the antibody or antigen binding fragment thereof is the 3E10 antibody. In some embodiments, the 3E10 comprises a heavy chain variable region of SEQ ID NO: 1023 or an analog or homolog comprising at least 85% sequence identity and being capable of binding cells and reaching the cytosol. In some embodiments, the 3E10 comprises a light chain variable region of SEQ ID NO: 1024 or an analog or homolog comprising at least 85% sequence identity and being capable of binding cells and reaching the cytosol. In some embodiments, the antibody or antigen binding fragment thereof is the 71F12 antibody. In some embodiments, the 71F12 comprises a heavy chain variable region of SEQ ID NO: 1026 or an analog or homolog comprising at least 85% sequence identity and being capable of binding cells and reaching the cytosol. In some embodiments, the 71F12 comprises a light chain variable region of SEQ ID NO: 1027 or an analog or homolog comprising at least 85% sequence identity and being capable of binding cells and reaching the cytosol.

In some embodiments, the antigen binding molecule comprises at least one immunogenic peptide. In some embodiments, the antigen binding molecule comprises at least 1, 2, 3, 4, 5, 6, 7, 8, 9 or 10 immunogenic peptides. Each possibility represents a separate embodiment of the invention. In some embodiments, the immunogenic peptide is an exogenous immunogenic peptide. In some embodiments, the immunogenic peptide is inserted into the antigen binding molecule. In some embodiments, the immunogenic peptide is inserted into a variable region of the antibody or antigen binding fragment thereof. In some embodiments, the immunogenic peptide is not a natural part of the antigen binding molecule. In some embodiments, the peptide is a sequence of amino acids. In some embodiments, the sequence of immunogenic amino acids is inserted into the sequence of the antigen binding molecule. In some embodiments, the immunogenic peptide replaces amino acids of the antigen binding molecule. In some embodiments, insertion of the immunogenic peptide comprises removal of amino acid sequence. In some embodiments, the amino acid sequence is sequence of the antibody or antigen binding fragment thereof. In some embodiments, the immunogenic peptide is not artificially linked to antigen binding molecule. In some embodiments, the antigen binding molecule is a recombinant molecule. In some embodiments, the recombinant molecule comprises an amino acid sequence of the immunogenic peptide. In some embodiments, the immunogenic peptide is not linked by a chemical linkage to the antigen binding molecule. In some embodiments, a chemical linkage is any linkage other than a peptide linkage. In some embodiments, a chemical linkage is any linkage other than an amino acid linkage. In some embodiments, the immunogenic peptide is linked to the antigen binding molecule by a peptide bond, an amino acid linkage or both.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the term “recombinant protein” refers to a protein which is coded for by a recombinant DNA and is thus not naturally occurring. The term “recombinant DNA” refers to DNA molecules formed by laboratory methods of genetic recombination. Generally, this recombinant DNA is in the form of a vector, plasmid or virus used to express the recombinant protein in a cell.

In some embodiments, the immunogenic peptide comprises at least 2, 3, 4, 5, 6, 7, 8, 9, or 10 amino acids. Each possibility represents a separate embodiment of the invention. In some embodiments, the immunogenic peptide comprises at most 5, 6, 7, 8, 9, 10, 11, 12, 13, 14, 15, 16, 17, 18, 19, or 20 amino acids. Each possibility represents a separate embodiment of the invention. In some embodiments, the immunogenic peptide is between 3 and 20, 3 and 15, 3 and 12, 3 and 11, 3 and 10, 3 and 7, 5 and 20, 5 and 15, 5 and 12, 5 and 11, 5 and 10, 7 and 20, 7 and 15, 7 and 12, 7 and 11, or 7 and 10 amino acids. Each possibility represents a separate embodiment of the invention. In some embodiments, the immunogenic peptide is between 8 and 11 amino acids. In some embodiments, the immunogenic peptide consists of 8 amino acids. In some embodiments, the immunogenic peptide consists of 9 amino acids. In some embodiments, the immunogenic peptide consists of 10 amino acids. In some embodiments, the immunogenic peptide consists of 11 amino acids.

As used herein, the term “immunogenic peptide” refers to an amino acids sequence that produces an immune response when exposed to the human immune system. In some embodiments, an immunogenic peptide is a non-human peptide. In some embodiments, the immunogenic peptide produces an immune response from an immune cell. In some embodiments, the immunogenic peptide is recognized an immune response from an immune cell. In some embodiments, recognized is bound by. In some embodiments, an immunogenic peptide produces an immune response from a dendritic cell. In some embodiments, an immunogenic peptide is displayed on the cell surface. In some embodiments, an immunogenic peptide is displayed as an MHC molecule. In some embodiments, an immunogenic peptide is displayed in complex with HLA. In some embodiments, the immunogenic peptide produces an immune response from a T cell. In some embodiments, the immune cell is a dendritic cell. In some embodiments, the immune cell is a T cell. In some embodiments, a T cell is selected from a CD4 T cell and a CD8 T cell. In some embodiments, the T cell is a CD4 T cell. In some embodiments, the T cell is a CD8 T cell. In some embodiments, the immunogenic peptide comprises a CD4 epitope, a CD8 epitope or both. In some embodiments, the immunogenic peptide produces an immune response from an NK cell. In some embodiments, an immune response is an elevated immune response. In some embodiments, immunogenic comprises increase immunogenicity. In some embodiments, increased is as compared to a control peptide. In some embodiments, the control peptide is a human peptide. In some embodiments, a control peptide is a non-cancerous peptide. In some embodiments, a control peptide is a non-immunogenic peptide.

In some embodiments, the immunogenic peptide is a cancer peptide. In some embodiments, the cancer peptide is a cancer specific peptide. In some embodiments, the cancer peptide is a cancer elevated peptide. In some embodiments, the cancer peptide is a peptide with increased surface expression in cancer cells. In some embodiments, a cancer peptide is a peptide provided in Table 1. In some embodiments, a cancer peptide is selected from a sequence provided in Table 1. In some embodiments, a cancer peptide is selected from the sequences provided in SEQ ID NO: 702-1020. Cancer peptides are well known in the art and can found, for example at the Cancer Antigenic Peptide Database: caped.icp.ucl.ac.be/Peptide/search.

TABLE 1
Cancer peptides

| | | SEQ ID | |
|---|---|---|---|
| Gene/Protein | Peptide Sequence | NO: | Position |
| p53 | SQKTYQGSY | 702 | 99-107 |
| NY-ESO-1/LAGE-2 | KEFTVSGNILTI | 703 | 124-135 |
| MAGE-A6 | MVKISGGPR | 704 | 290-298 |
| Melan-A/MART-1 | AEEAAGIGIL(T) | 705 | 24-33(34) |
| MAGE-A1 | DPARYEFLW | 706 | 258-266 |
| MAGE-A10 | DPARYEFLW | 707 | 290-298 |
| MAGE-A1 | REPVTKAEML | 708 | 120-129 |
| MAGE-A2 | REPVTKAEML | 709 | 127-136 |
| MAGE-A3 | REPVTKAEML | 710 | 127-136 |
| MAGE-A4 | SESLKMIF | 711 | 156-163 |
| MAGE-A6 | REPVTKAEML | 712 | 127-136 |
| PBF | CTACRWKKACQR | 713 | 499-510 |
| HSDL1 | CYMEAVAL | 714 | 20-27 |
| LAGE-1 | LAAQERRVPR | 715 | ORF2 |
| LAGE-1 | LAAQERRVPR | 716 | (18-27) |
| NY-ESO-1/LAGE-2 | ASGPGGGAPR | 717 | 53-62 |
| NY-ESO-1/LAGE-2 | LAAQERRVPR | 718 | ORF2 |
| NY-ESO-1/LAGE-2 | LAAQERRVPR | 719 | (18-27) |
| TRP-1/gp75 | MSLQRQFLR | 720 | alt. ORF |
| TRP-2 | LLGPGRPYR | 721 | 197-205 |
| TRP-2 | LLGPGRPYR | 722 | 197-205 |
| TRP-2 | LLGPGRPYR | 723 | 197-205 |
| SNRPD1 | SHETVIIEL | 724 | 43739 |
| tyrosinase | LHHAFVDSIF | 725 | 388-397 |
| NFYC | QQITKTEV | 726 | 275-282 |
| MAGE-A3 | WQYFFPVIF | 727 | 143-151 |
| NY-ESO-1/LAGE-2 | FATPMEAEL | 728 | 96-104 |
| GAGE-3,4,5,6,7 | YYWPRPRRY | 729 | 43374 |
| NA88-A | QGQHFLQKV | 730 | |
| MAGE-A3 | MEVDPIGHLY | 731 | 167-176 |
| MAGE-A3 | AELVHFLLL | 732 | 114-122 |
| VEGF | SRFGGAVVR | 733 | -i |
| BAGE-1 | AARAVFLAL | 734 | 44471 |
| MAGE-A1 | SAYGEPRKL | 735 | 230-238 |
| MAGE-A6 | ISGGPRISY | 736 | 293-301 |
| K-ras | GADGVGKSA | 737 | 43374 |
| K-ras | GADGVGKSAL | 738 | 43739 |
| gp100/Pmel17 | SNDGPTLI | 739 | 71-78 |
| TRP-2 | ANDPIFVVL | 740 | 387-395 |
| tyrosinase | QCSGNFMGF | 741 | 90-98 |
| gp100/Pmel17 | RTKQLYPEW | 742 | 40-42 and |
| | | | 47-52 |
| Elongation factor 2 | ETVSEQSNV | 743 | 581-589 |
| MUM-3 | EAFIQPITR | 744 | 322-330 |
| LAGE-1 | ELVRRILSR | 745 | 103-111 |
| MAGE-A1 | EVYDGREHSA | 746 | 222-231 |
| NY-ESO-1/LAGE-2 | TVSGNILTIR | 747 | 127-136 |
| TRP2-INT2 | EVISCKLIKR | 748 | intron 2 |
| gp100/Pmel17 | HTMEVTVYHR | 749 | 182-191 |
| MAGE-A1 | ITKKVADLVGF | 750 | 102-112 |
| MAGE-C2 | ASSTLYLVF | 751 | 42-50 |
| MAGE-A1 | SAFPTTINF | 752 | 62-70 |
| NY-ESO-1/LAGE-2 | MPFATPMEA | 753 | 94-102 |
| NY-ESO-1/LAGE-2 | FATPMEAELAR | 754 | 96-106 |
| CDK12 | CILGKLFTK | 755 | 924-932 |
| CDKN2A | AVCPWTWLR | 756 | 125-133 |
| | | | (p14ARF-ORF3) |
| MATN | KTLTSVFQK | 757 | 226-234 |
| HERV-E | ATFLGSLTWK | 758 | |
| gp100/Pmel17 | ALNFPGSQK | 759 | 87-95 |
| KK-LC-1 | RQKRILVNL | 760 | 76-84 |
| BCR-ABL fusion protein (b3a2) | GFKQSSKAL | 761 | 922-930 |
| TAG-1 | LSRLSNRLL | 762 | 42-50 |
| TAG-2 | LSRLSNRLL | 763 | 42-50 |
| RBAF600 | RPHVPESAF | 764 | 329-337 |
| SYT-SSX1 or -SSX2 fusion | QRPYGYDQIM | 765 | 402-410 |
| protein | | | (SYT) |
| LAGE-1 | APRGVRMAV | 766 | ORF2 |
| LAGE-1 | APRGVRMAV | 767 | (46-54) |
| MAGE-A1 | RVRFFFPSL | 768 | 289-298 |
| NY-ESO-1/LAGE-2 | APRGPHGGAASGL | 769 | 60-72 |
| gp100/Pmel17 | SSPGCQPPA | 770 | 529-537 |
| Intestinal carboxyl esterase | SPRWWPTCL | 771 | alt. ORF |
| RAGE-1 | SPSSNRIRNT | 772 | 44136 |
| RU2AS | LPRWPPPQL | 773 | antisense |
| MAGE-A1 | SAYGEPRKL | 774 | 230-238 |
| NY-ESO-1/LAGE-2 | LAMPFATPM | 775 | 92-100 |
| MUM-2 | FRSGLDSYV | 776 | 126-134 |
| GAGE-1,2,8 | YRPRPRRY | 777 | 42614 |
| NY-ESO-1/LAGE-2 | ARGPESRLL | 778 | 80-88 |
| beta-catenin | SYLDSGIHF | 779 | 29-37 |
| KM-HN-1 | NYNNFYRFL | 780 | 196-204 |
| KM-HN-1 | EYSKECLKEF | 781 | 499-508 |
| KM-HN-1 | EYLSLSDKI | 782 | 770-778 |
| LY6K | RYCNLEGPPI | 783 | 119-128 |
| MAGE-A2 | EYLQLVFGI | 784 | 156-164 |
| MAGE-A3 | TFPDLESEF | 785 | 97-105 |
| MAGE-A3 | VAELVHFLL | 786 | 113-121 |
| MAGE-A4 | NYKRCFPVI | 787 | 143-151 |
| MAGE-A4 | NYKRCFPVI | 788 | 143-151 |
| NY-ESO-1/LAGE-2 | YLAMPFATPME | 789 | 91-101 |
| SAGE | LYATVIHDI | 790 | 715-723 |
| CEA | TYACFVSNL | 791 | 652-660 |
| CEA | QYSWFVNGTF | 792 | 268-277 |
| gp100/Pmel17 | VYFFLPDHL | 793 | intron 4 |
| OA1 | LYSACFWWL | 794 | 126-134 |
| tyrosinase | IYMDGTADFSF | 795 | 368-373 and |
| | | | 336-340e |
| tyrosinase | AFLPWHRLF | 796 | 206-214 |
| CD45 | KFLDALISL | 797 | 556-564 |
| EpCAM | RYQLDPKFI | 798 | 173-181 |
| EZH2 | KYDCFLHPF | 799 | 291-299 |
| EZH2 | KYVGIEREM | 800 | 735-743 |
| glypican-3 | EYILSLEEL | 801 | 298-306 |
| HER-2/neu | TYLPTNASL | 802 | 63-71 |
| HSPH1 | NYGIYKQDL | 803 | 180-188 |
| MUC5AC | TCQPTCRSL | 804 | 716-724 |
| PRAME | LYVDSLFFLc | 805 | 301-309 |
| PSMA | NYARTEDFF | 806 | 178-186 |
| RNF43 | NSQPVWLCL | 807 | 721-729 |
| WT1 | CMTWNQMNL | 808 | 235-243 |
| CASP-8 | FPSDSWCYF | 809 | 476-484 |
| K-ras | VVVGAVGVG | 810 | 42186 |
| MAGE-A1 | EADPTGHSY | 811 | 161-169 |
| MAGE-A3 | EVDPIGHLY | 812 | 168-176 |
| MAGE-A6 | EVDPIGHVY | 813 | 168-176 |
| NY-ESO-1/LAGE-2 | MPFATPMEAEL | 814 | 94-104 |
| gp100/Pmel17 | VPLDCVLYRY | 815 | 471-480 |
| gp100/Pmel17 | LPHSSSHWL | 816 | 630-638 |
| Melan-A/MART-1 | EAAGIGILTV | 817 | 26-35 |
| tyrosinase | LPSSADVEF | 818 | 312-320 |
| tyrosinase | TPRLPSSADVEF | 819 | 309-320 |
| M-CSF | LPAVVGLSPGEQEY | 820 | alt. ORF |
| KIAAO205 | AEPINIQTW | 821 | 262-270 |
| MUM-1 | EEKLIVVLF | 822 | 30-38 |
| MUM-2 | SELFRSGLDSY | 823 | 123-133 |
| OS-9 | KELEGILLL | 824 | 438-446 |
| MAGE-A1 | KEADPTGHSY | 825 | 160-169 |
| MAGE-A3 | MEVDPIGHLY | 826 | 167-176 |
| MAGE-C2 | SESIKKKVL | 827 | 307-315 |
| tyrosinase | SEIWRDIDFD | 828 | 192-200 |
| EFTUD2 | KILDAVVAQK | 829 | 668-677 |
| GPNMB | TLDWLLQTPK | 830 | 179-188 |
| Myosin class I | KINKNPKYK | 831 | 911-919 |
| SIRT2 | KIFSEVTLK | 832 | 192-200 |
| MAGE-A1 | SLFRAVITK | 833 | 96-104 |
| CEA | HLFGYSWYK | 834 | 61-69 |
| gp100/Pmel17 | LIYRRRLMK | 835 | 614-622 |
| gp100/Pmel17 | IALNFPGSQK | 836 | 86-95 |
| gp100/Pmel17 | ALLAVGATK | 837 | 17-25 |
| gp100/Pmel17 | RSYVPLAHR | 838 | 195-202 and |
| | | | 191 or 192e |
| gp100/Pmel17 | ALNFPGSQK | 839 | 87-95 |
| mammaglobin-A | PLLENVISK | 840 | 23-31 |
| FGF5 | NTYASPRFKf | 841 | 172-176 and |
| | | | 217-220 |
| HER-2/neu | VLRENTSPK | 842 | 754-762 |
| MMP-7 | SLFPNSPKWTSK | 843 | 96-107 |
| RGS5 | GLASFKSFLK | 844 | 74-83 |
| RhoC | RAGLQVRKNK | 845 | 176-185 |
| FLT3-ITD | YVDFREYEYY | 846 | 591-600 |
| MART2 | FLEGNEVGKTY | 847 | 446-455 |
| N-ras | ILDTAGREEY | 848 | 55-64 |
| PPP1R3B | YTDFHCQYV | 849 | 172-180 |
| MAGE-A1 | EADPTGHSY | 850 | 161-169 |
| MAGE-A3 | EVDPIGHLY | 851 | 168-176 |
| MAGE-A4 | EVDPASNTY | 852 | 169-177 |
| Sp17 | ILDSSEEDK | 853 | 103-111 |
| tyrosinase | KCDICTDEY | 854 | 243-251 |
| tyrosinase | SSDYVIPIGTY | 855 | 146-156 |
| AIM-2 | RSDSGQQARY | 856 | intron |
| WT1 | TSEKRPFMCAY | 857 | 317-327 |
| MAGE-A1 | RVRFFFPSL | 858 | 289-298 |
| MAGE-A12 m | VRIGHLYIL | 859 | 170-178 |
| MAGE-A12 m | VRIGHLYIL | 860 | 170-178 |
| MAGE-A12 m | EGDCAPEEK | 861 | 212-220 |
| MAGE-A2 | EGDCAPEEK | 862 | 212-220 |
| MAGE-A3 | EGDCAPEEK | 863 | 212-220 |
| MAGE-A6 | EGDCAPEEK | 864 | 212-220 |
| Melan-A/MART-1 | RNGYRALMDKS | 865 | 51-61 |
| alpha-actinin-4 | FIASNGVKLV | 866 | 118-127 |
| BCR-ABL fusion protein (b3a2) | SSKALQRPV | 867 | 926-934 |
| CASP-5 | FLIIWQNTM | 868 | 67-75 |
| CDK4 | ACDPHSGHFV | 869 | 23-32 |
| CLPP | ILDKVLVHL | 870 | 240-248 |
| CSNK1A1 | GLFGDIYLA | 871 | 26-34 |
| ETV6-AML1 fusion protein | RIAECILGM | 872 | 334-342 |
| FNDC3B | VVMSWAPPV | 873 | 292-300 |
| GAS7 | SLADEAEVYL | 874 | 141-150 |
| HAUS3 | ILNAMIAKI | 875 | 154-162 |
| hsp70-2 | SLFEGIDIYT | 876 | 286-295 |
| ME1 | FLDEFMEGV | 877 | 224-232 |
| OGT | SLYKFSPFPL | 878 | 28-37 |
| p53 | VVPCEPPEV | 879 | 217-225 |
| PRDX5 | LLLDDLLVSI | 880 | 163-172 |
| TGF-betaRII | RLSSCVPVA | 881 | 131-139 |
| TP53 | VVPCEPPEV | 882 | 217-225 |
| CT37/FMRINB | YLCSGSSYFV | 883 | 89-98 |
| Cyclin-A1 | SLIAAAAFCLA | 884 | 341-351 |
| Cyclin-A1 | FLDRFLSCM | 885 | 227-235 |
| GnTV | VLPDVFIRC(V) | 886 | intron |
| HERV-K-MEL | MLAVISCAV | 887 | 44440 |
| LAGE-1 | MLMAQEALAFL | 888 | ORF2 |
| LAGE-1 | MLMAQEALAFL | 889 | (1-11) |
| LAGE-1 | SLLMWITQC | 890 | 157-165 |
| LRPAP1 | FLGPWAAS | 891 | 21-30 |
| MAGE-A1 | KVLEYVIKV | 892 | 278-286 |
| MAGE-A1 | KVLEYVIKV | 893 | 278-286 |
| MAGE-A10 | GLYDGMEHL | 894 | 254-262 |
| MAGE-A12m | FLWGPRALV | 895 | 271-279 |
| MAGE-A2 | YLQLVFGIEV | 896 | 157-166 |
| MAGE-A3 | KVAELVHFL | 897 | 112-120 |
| MAGE-A3 | FLWGPRALV | 898 | 271-279 |
| MAGE-A4 | GVYDGREHTV | 899 | 230-239 |
| MAGE-A9 | ALSVMGVYV | 900 | 223-231 |
| MAGE-C1 | ILFGISLREV | 901 | 959-968 |
| MAGE-C1 | KVVEFLAML | 902 | 1083-1091 |
| MAGE-C2 | ALKDVEERV | 903 | 336-344 |
| MAGE-C2 | LLFGLALIEV | 904 | 191-200 |
| NY-ESO-1/LAGE-2 | SLLMWITQC | 905 | 157-165 |
| NY-ESO-1/LAGE-2 | SLLMWITQC | 906 | 157-165 |
| NY-ESO-1/LAGE-2 | SLLMWITQC | 907 | 157-165 |
| NY-ESO-1/LAGE-2 | MLMAQEALAFL | 908 | ORF2 |
| NY-ESO-1/LAGE-2 | MLMAQEALAFL | 909 | (1-11) |
| SSX-2 | KASEKIFYV | 910 | 41-49 |
| TAG-1 | SLGWLFLLL | 911 | 78-86 |
| XAGE-1b/GAGED2a | ROKKIRIQL | 912 | 21-29 |
| CEA | YLSGANLNL | 913 | 605-613 |
| CEA | GVLVGVALI | 914 | 694-702 |
| CEA | IMIGVLVGV | 915 | 691-699 |
| gp100/Pmel17 | KTWGQYWQV | 916 | 154-162 |
| gp100/Pmel17 | (A)MLGTHTMEV | 917 | 177(8)-186 |
| gp100/Pmel17 | KTWGQYWQV | 918 | 154-163 |
| gp100/Pmel17 | ITDQVPFSV | 919 | 209-217 |
| gp100/Pmel17 | YLEPGPVTA | 920 | 280-288 |
| gp100/Pmel17 | VLYRYGSFSV | 921 | 476-485 |
| gp100/Pmel17 | LLDGTATLRL | 922 | 457-466 |
| gp100/Pmel17 | RLMKQDFSV | 923 | 619-627 |
| gp100/Pmel17 | SLADTNSLAV | 924 | 570-579 |
| gp100/Pmel17 | RLPRIFCSC | 925 | 639-647 |
| Melan-A/MART-1 | ILTVILGVL | 926 | 32-40 |
| Melan-A/MART-1 | EAAGIGILTV | 927 | 26(27)-35 |
| NY-BR-1 | SLSKILDTV | 928 | 904-912 |
| PAP | TLMSAMTNL | 929 | 112-120 |
| PAP | ALDVYNGLL | 930 | 299-307 |
| PAP | FLFLLFFWL | 931 | 18-26 |
| PSA | FLTPKKLQCV | 932 | 165-174 |
| PSA | VISNDVCAQV | 933 | 178-187 |
| RAB38/NY-MEL-1 | VLHWDPETV | 934 | 50-58 |
| TRP-2 | SVYDFFVWL | 935 | 180-188 |
| TRP-2 | TLDSQVMSL | 936 | 360-368 |
| tyrosinase | MLLAVLYCL | 937 | 44440 |
| tyrosinase | CLLWSFQTSA | 938 | 42948 |
| tyrosinase | YMDGTMSQV | 939 | 369-377 |
| tyrosinase | YMDGTMSQV | 940 | 369-377 |
| adipophilin | SVASTITGV | 94 | 129-137 |
| ALDH1A1 | LLYKLADLI | 942 | 88-96 |
| alpha-foetoprotein (AFP) | GVALQTMKQ | 943 | 542-550 |
| alpha-foetoprotein (AFP) | FMNKFIYEI | 944 | 158-166 |
| BCLX (L) | YLNDHLEPWI | 945 | 173-182 |
| BING-4 | CQWGRLWQL | 946 | ORF2 |
| CALCA | VLLQAGSLHA | 947 | 16-25 |
| CALCA | FLALSILVL | 948 | 42979 |
| CALCA | LLAALVQDYL | 949 | 50-59 |
| CALCA | CMLGTYTQDF | 950 | 91-100 |
| CD274 | LLNAFTVTV | 95 | 15-23 |
| CPSF | LMLQNALTTM | 952 | 1360-1369 |
| CPSF | KVHPVIWSL | 953 | 250-258 |
| cyclin D1 | LLGATCMFV | 954 | 101-109 |
| DKK1 | ALGGHPLLGV | 955 | 20-29 |
| DKK1 | ALGGHPLLGV | 956 | 20-30 |
| ENAH (hMena) | TMNGSKSPV | 957 | 502-510 |
| EZH2 | FMVEDETVL | 958 | 120-128 |
| EZH2 | FINDEIFVEL | 959 | 165-174 |
| G250/MN/CAIX | HLSTAFARV | 960 | 254-262 |
| glypican-3 | FVGEFFTDV | 961 | 144-152 |
| glypican-3 | TIHDSIQYV | 962 | 325-334 |
| HEPACAM | RLAPFVYLL | 963 | 16-24 |
| Hepsin | SLLSGDWVL | 964 | 191-199 |
| Hepsin | GLQLGVQAV | 965 | 229-237 |
| Hepsin | PLTEYIQPV | 966 | 268-276 |
| HER-2/neu | KIFGSLAFL | 967 | 369-377 |
| HER-2/neu | IISAVVGIL | 968 | 654-662 |
| HER-2/neu | ALCRWGLLL | 969 | 44329 |
| HER-2/neu | ILHNGAYSL | 970 | 435-443 |
| HER-2/neu | VVLGVVFGI | 971 | 665-673 |
| HER-2/neu | YMIMVKCWMI | 972 | 952-961 |
| HER-2/neu | YLVPQQGFFC | 973 | 1023-1032 |
| HER-2/neu | RLLQETELV | 974 | 689-697 |
| HER-2/neu | HLYQGCQVV | 975 | 48-56 |
| HER-2/neu | PLQPEQLQV | 976 | 391-399 |
| HER-2/neu | ALIHHNTHL | 977 | 466-474 |
| HER-2/neu | TLEEITGYL | 978 | 402-410 |
| HER-2/neu | PLTSIISAV | 979 | 650-658 |
| HLA-DOB | FLLGLIFLL | 980 | 232-240 |
| HSPH1 | RLMNDMTAV | 981 | 169-177 |
| IDO1 | ALLEIASCL | 982 | 199-207 |
| IGF2B3 | NLSSAEVVV | 983 | 515-523 |
| IL13Ralpha2 | WLPFGFILI | 984 | 345-353 |
| Kallikrein 4 | FLGYLILGV | 985 | 43770 |
| KIF20A | AQPDTAPLPV | 986 | 284-293 |
| KIF20A | LLSDDDVVV | 987 | 44166 |
| KIF20A | CIAEQYHTV | 988 | 809-817 |
| Lengsin | FLPEFGISSA | 989 | 270-279 |
| mdm-2 | VLFYLGQY | 990 | 53-60 |
| Meloe | TLNDECWPA | 991 | 36-44 |
| Midkine | ALLALTSAV | 992 | 13-21 |
| Midkine | AQCQETIRV | 993 | 114-122 |
| MMP-2 | GLPPDVQRVh | 994 | 560-568 |
| MUC1 | STAPPVHNV | 995 | 950-958 |
| MUC1 | LLLLTVLTV | 996 | 44166 |
| nectin-4 | VLVPPLPSL | 997 | 145-153 |
| p53 | RMPEAAPPV | 998 | 65-73 |
| p53 | LLGRNSFEV | 999 | 264-272 |
| PAX5 | TLPGYPPHV | 1000 | 311-319 |
| PLAC1 | VLCSIDWFM | 1001 | 28-36 |
| PRAME | VLDGLDVLL | 1002 | 100-108 |
| PRAME | ALYVDSLFFL | 1003 | 300-309 |
| PRAME | SLYSFPEPEA | 1004 | 142-151 |
| PRAME | SLLQHLIGL | 1005 | 425-433 |
| RAGE-1 | LKLSGVVRL | 1006 | 352-360 |
| RAGE-1 | PLPPARNGGLg | 1007 | 32-40 |
| RGS5 | LAALPHSCL | 1008 | 41395 |
| RNF43 | ALWPWLLMA(T) | 1009 | 11-19(20) |
| Secernin 1 | KMDAEHPEL | 1010 | 196-204 |
| SOX10 | AWISKPPGV | 1011 | 332-340 |
| SOX10 | SAWISKPPGV | 1012 | 331-340 |
| STEAP1 | MIAVFLPIV | 1013 | 292-300 |
| STEAP1 | HQQYFYKIPILVINK | 1014 | 102-116 |
| survivin | ELTLGEFLKL | 1015 | 95-104 |
| survivin | ELTLGEFLKL | 1016 | 95-104 |
| Telomerase | ILAKFLHWLE | 1017 | 540-548 |
| Telomerase | RLVDDFLLV | 1018 | 865-873 |
| TPBG | RLARLALVL | 1019 | 17-25 |
| IGF2B3 | RLLVPTQFV | 1020 | 199-207 |

In some embodiments, the immunogenic peptide is a non-human peptide. In some embodiments, the immunogenic peptide is a viral peptide. In some embodiments, the immunogenic peptide is a bacterial peptide. Viral peptides are well known in the art and any such peptide may be employed. Such peptides can found, for example, at the Immune Epitope Database (IEDB) and VDJDB database: iedb.org and vdjdb.cdr3.net. In some embodiments, the viral peptide is derived from a virus selected from Cytomegalovirus (CMV), Epstein-Barr virus (EBV) or Influenza virus (FLU). In some embodiments, the viral peptide is derived from a virus selected from CMV, EBV, FLU, Severe acute respiratory syndrome coronavirus 2 (SARS-CoV2), Adenovirus and Human Papilloma virus (HPV). In some embodiments, the virus is CMV. In some embodiments, the virus is EBV. In some embodiments, the virus is FLU. In some embodiments, the virus is SARS-CoV2. In some embodiments, the virus is Adenovirus. In some embodiments, the virus is HPV. In some embodiments, the viral peptide is a peptide provided in Table 2. In some embodiments, the viral peptide is selected from a sequence provided in Table 2. In some embodiments, the viral peptide is a peptide provided in Table 3. In some embodiments, the viral peptide is selected from a sequence provided in Table 3. In some embodiments, the viral peptide is selected from SEQ ID NO: 1-701. In some embodiments, the viral peptide is selected from SEQ ID NO: 1-695. In some embodiments, the viral peptide is selected from SEQ ID NO: 1-11. In some embodiments, the viral peptide is selected from SEQ ID NO: 1-9. In some embodiments, the viral peptide is selected from SEQ ID NO: 1-5. In some embodiments, the viral peptide is SEQ ID NO: 1. In some embodiments, the viral peptide is SEQ ID NO: 2. In some embodiments, the viral peptide is SEQ ID NO: 3. In some embodiments, the viral peptide is SEQ ID NO: 4. In some embodiments, the viral peptide is SEQ ID NO: 5. In some embodiments, the viral peptide is SEQ ID NO: 6. In some embodiments, the viral peptide is SEQ ID NO: 7. In some embodiments, the viral peptide is SEQ ID NO: 8. In some embodiments, the viral peptide is SEQ ID NO: 9. In some embodiments, the viral peptide is SEQ ID NO: 10. In some embodiments, the viral peptide is SEQ ID NO: 11. In some embodiments, the viral peptide is selected from SEQ ID NO: 1, 2, 3, 4, 5, 151, 197, 471, 677, 696, 697, 698, 699, 700, and 701.

TABLE 2
Viral peptides

| Peptide | SEQ ID NO: | source |
|---|---|---|
| NLVPMVATV | 1 | CMV |
| GLCTLVAML | 197 | EBV |
| CLGGLLTMV | 2 | EBV |
| CTELKLSDY | 677 | FLU |
| CLGGLLTMV, GLCTLVAML, NLVPMVATV, | 2, 197, 1, 3 | EBV, CMV, |
| GILGFVFTL | | FLU |
| DYNFVKQLF, GLCTLVAML, TYPVLEEMF, | 696, 197, 697, | EBV, CMV, |
| RYSIFFDYM, AVFDRKSDAK, TYSAGIVQI, | 698, 4, 699, | FLU |
| IVTDFSVIK, SSCSSCPLSK, AYAQKIFKIL, | 151, 629, 471, | |
| VYALPLKML, QYDPVAALF, NLVPMVATV, | 700, 5, 1, 701 | |
| GPISGHVLK, | | |

TABLE 3
Full list of viral peptides

| | SEQ ID |
|---|---|
| Peptide | NO: |
| NLVPMVATV | 1 |
| CLGGLLTMV | 2 |
| GILGFVFTL | 3 |
| AVFDRKSDAK | 4 |
| QYDPVAALF | 5 |
| TPSVSSSISSL | 6 |
| LPFNDGVYF | 7 |
| FLGERVTLT | 8 |
| LLALHRSYL | 9 |
| STDVASLNY | 10 |
| TLGIVCPI | 11 |
| RYSIFFDY | 12 |
| CPLSKILL | 13 |
| SLGYITTV | 14 |
| RAKFKQLL | 15 |
| TNKIKEQL | 16 |
| QTRQKFHL | 17 |
| EMRLRMIL | 18 |
| MAREKNDL | 19 |
| CCKCDSTL | 20 |
| HDIILECV | 21 |
| LPQGFSAL | 22 |
| NLTTRTQL | 23 |
| AEHVNNSY | 24 |
| AEVQIDRL | 25 |
| EPLVDLPI | 26 |
| FPREGVFV | 27 |
| NITRFQTL | 28 |
| QPYRVVVL | 29 |
| RLQSLQTY | 30 |
| SPRRARSV | 31 |
| VYYPDKVF | 32 |
| YAWNRKRI | 33 |
| FLPFFSNV | 34 |
| APHGVVFL | 35 |
| GVYHKNNK | 36 |
| TYFNLGNKF | 37 |
| TDLGQNLLY | 38 |
| TLLYVLFEV | 39 |
| VLAWTRAFV | 40 |
| YVAGFLALY | 41 |
| ALMGAVTSL | 42 |
| MMLRDRWSL | 43 |
| AVLCLYLLY | 44 |
| FLWEDQTLL | 45 |
| FIPQYLSAV | 46 |
| FLIAYQPLL | 47 |
| SVYPYDEFV | 48 |
| VYMSPFYGY | 49 |
| FLGDDPSPA | 50 |
| RLTGYPAGI | 51 |
| APASVYQPA | 52 |
| CPRRPAVAF | 53 |
| VVRGPTVSL | 54 |
| LIDGIFLRY | 55 |
| SQLAHLVYV | 56 |
| FLGAGALAV | 57 |
| FLGGHVAVA | 58 |
| TLRGLFFSV | 59 |
| KYFYCNSLF | 60 |
| EYQRLYATF | 61 |
| APRIGGRRA | 62 |
| APRTWCRLL | 63 |
| ALMLRLLRI | 64 |
| RILGVLVHL | 65 |
| YMESVFQMY | 66 |
| ILIEGIFFA | 67 |
| YMANQILRY | 68 |
| VPRPDDPVL | 69 |
| VYTPSPYVF | 70 |
| AYLPRPVEF | 71 |
| AILTQYWKY | 72 |
| ATDSLNNEY | 73 |
| AYVSVLYRW | 74 |
| LASDPHYEY | 75 |
| LLAYVSVLY | 76 |
| RLNELLAYV | 77 |
| SIVHHHAQY | 78 |
| ALATVTLKY | 79 |
| ALLDRDCRV | 80 |
| FLADAVVRL | 81 |
| FTAPEVGTY | 82 |
| RLLGFADTV | 83 |
| RSSLGSLLY | 84 |
| ALHTALATV | 85 |
| TLLELVVSV | 86 |
| VPGWSRRTL | 87 |
| ALLAKMLFY | 88 |
| RMLGDVMAV | 89 |
| TMLEDHEFV | 90 |
| ALLGLTLGV | 91 |
| FVLATGDFV | 92 |
| GIFEDRAPV | 93 |
| LLTTPKFTV | 94 |
| NLLTTPKFT | 95 |
| TMYYKDVTV | 96 |
| YLANGGFLI | 97 |
| ALSALLTKL | 98 |
| FLTCTDRSV | 99 |
| DRLDNRLQL | 100 |
| KSRRPLTTF | 101 |
| RRAQMAPKR | 102 |
| GPHETITAL | 103 |
| PAWSRRTLL | 104 |
| ASDSLNNEY | 105 |
| FLVDAIVRV | 106 |
| GLADTVVAC | 107 |
| RPRGEVRFL | 108 |
| SAPLPSNRV | 109 |
| SLPRSRTPI | 110 |
| ALWALPHAA | 111 |
| ILIEGIFFV | 112 |
| VTEHDTLLY | 113 |
| ELNRKMIYM | 114 |
| IPSINVHHY | 115 |
| TRATKMQVI | 116 |
| QIKVRVKMV | 117 |
| DELRRKMMY | 118 |
| EEAIVAYTL | 119 |
| ELKRKMMYM | 120 |
| QIKVRVDMV | 121 |
| VLEETSVML | 122 |
| ARAKKDELK | 123 |
| ARAKKDELR | 124 |
| DELKRKMIY | 125 |
| FMDILTTCV | 126 |
| KEWAYCVEM | 127 |
| LITGRLAAL | 128 |
| LLLNCLWSV | 129 |
| TMLDIQPED | 130 |
| LPRWYFYYL | 131 |
| KLWHYCSTL | 132 |
| LLIEGIFFI | 133 |
| AFLGERVTL | 134 |
| KLGPGEEQV | 135 |
| RFIAQLLLL | 136 |
| TLTSYWRRV | 137 |
| VEDLFGANL | 138 |
| WQWEHIPPA | 139 |
| LPCVLWPVL | 140 |
| RAKFKQLLQ | 141 |
| SENDRLRLL | 142 |
| TLDTKPLSV | 143 |
| FMVFLQTHI | 144 |
| HPVGEADYF | 145 |
| VLKDAIKDL | 146 |
| AYSSWMYSY | 147 |
| QAKWRLQTL | 148 |
| RLRAEAQVK | 149 |
| SVRDRLARL | 150 |
| IVTDFSVIK | 151 |
| VSFIEFVGW | 152 |
| EGGVGWRHW | 153 |
| QPRAPIRPI | 154 |
| RRIYDLIEL | 155 |
| RPPIFIRLL | 156 |
| YPLHEQHGM | 157 |
| LMIIPLINV | 158 |
| SLVIVTTFV | 159 |
| TLFIGSHVV | 160 |
| YLQQNWWTL | 161 |
| ALLVLYSFA | 162 |
| GLGTLGAAL | 163 |
| LLSAWILTA | 164 |
| LLWTLVVLL | 165 |
| MGSLEMVPM | 166 |
| YLLEMLWRL | 167 |
| VLQWASLAV | 168 |
| FLYALALLL | 169 |
| IEDPPFNSL | 170 |
| RRRWRRLTV | 171 |
| TYGPVFMCL | 172 |
| FLRGRAYGL | 173 |
| LVLILYLCV | 174 |
| LLNGWRWRL | 175 |
| IGLITVLFL | 176 |
| DAAPAIQHI | 177 |
| KQYLGVYIW | 178 |
| TYATFLVTW | 179 |
| KENIAAYKF | 180 |
| KTNNWHAGW | 181 |
| APKTATSSW | 182 |
| QTTGRITNR | 183 |
| VEDINRVFL | 184 |
| CYDHAQTHL | 185 |
| TLDYKPLSV | 186 |
| YRSGIIAVV | 187 |
| RPPIFIRRL | 188 |
| FLDKGTYTL | 189 |
| ARYAYYLQF | 190 |
| RRRKGWIPL | 191 |
| LQHYREVAA | 192 |
| AENAGNDAC | 193 |
| RVRAYTYSK | 194 |
| YVLDHLIVV | 195 |
| DEVEFLGHY | 196 |
| GLCTLVAML | 197 |
| KDTWLDARM | 198 |
| VLFGLLCLL | 199 |
| ELRRKMMYM | 200 |
| ELKRKMIYM | 201 |
| EFKSKFSTL | 202 |
| EGRDRILTV | 203 |
| FEKERFLFL | 204 |
| EGRERILTV | 205 |
| NVKHKKNPL | 206 |
| VVKGKVLSI | 207 |
| EARRRLAEM | 208 |
| RSKPRHMCV | 209 |
| DFKSKYLTL | 210 |
| SPRSRLQQL | 211 |
| VLATAVREL | 212 |
| FQANTPPAV | 213 |
| IPYTAAVQV | 214 |
| KLAKLIIDL | 215 |
| RLPREKLKK | 216 |
| SPKAGLLSL | 217 |
| SLQQEITLL | 218 |
| ISDYFHNTY | 219 |
| KVLIRCYLC | 220 |
| SIDQLCKTF | 221 |
| HNNGICWGN | 222 |
| CYEQLGDSS | 223 |
| ITIRCIICQ | 224 |
| KTLEERVKK | 225 |
| MRGDKATIK | 226 |
| PYGVCIMCL | 227 |
| QLGDSSDEE | 228 |
| RLQCVQCKK | 229 |
| VYKFLFTDL | 230 |
| YYYAGSSRL | 231 |
| LQFIFQLCK | 232 |
| MTLCAEVKK | 233 |
| QYRVFRIKL | 234 |
| RIKLPDPNK | 235 |
| ILIRCIICQ | 236 |
| KCLNEILIR | 237 |
| KVCLRLLSK | 238 |
| ATEVRTLQQ | 239 |
| CTIVCPSCA | 240 |
| LCINSTATE | 241 |
| AVPDDLYIK | 242 |
| KYTFWEVNL | 243 |
| RVRLPDPNK | 244 |
| TSESQLFNK | 245 |
| YTFWEVNLK | 246 |
| YLTAPTGCI | 247 |
| DSAPILTAF | 248 |
| KSAIVTLTY | 249 |
| LAVSKNKAL | 250 |
| LQDVSLEVY | 251 |
| NPCHTTKLL | 252 |
| NTTPIVHLK | 253 |
| QVILCPTSV | 254 |
| RLECAIYYK | 255 |
| SPEIIRQHL | 256 |
| TLYTAVSST | 257 |
| VVEGQVDYY | 258 |
| YRFKKHCTL | 259 |
| ALQAIELQL | 260 |
| TLQDVSLEV | 261 |
| YIIFVYIPL | 262 |
| FAFRDLCIV | 263 |
| IILECVYCK | 264 |
| TTLEQQYNK | 265 |
| VCDKCLKFY | 266 |
| CPEEKQRHL | 267 |
| YGTTLEQQY | 268 |
| EYRHYCYSL | 269 |
| VYDFAFQDL | 270 |
| GTLGIVCPI | 271 |
| IVCPICSQK | 272 |
| QAEPDRAHY | 273 |
| RAHYNIVTF | 274 |
| TLHEYMLDL | 275 |
| LQPETTDLY | 276 |
| TPTLHEYML | 277 |
| LLMGTLGIV | 278 |
| TLGIVCPIC | 279 |
| IHSMNSTIL | 280 |
| ISEYRHYCY | 281 |
| KFYSKISEY | 282 |
| KLPQLCTEL | 283 |
| VYDFAFRDL | 284 |
| TIHDIILEC | 285 |
| YMLDLQPET | 286 |
| IHSMNSSIL | 287 |
| NVFPIFLQM | 288 |
| KLPDLCTEL | 289 |
| NLLIRCLRC | 290 |
| FQQLFLNTL | 291 |
| LFLNTLSFV | 292 |
| LLLGTLNIV | 293 |
| TIDQLCKTF | 294 |
| EADVQQWLT | 295 |
| FYTPLADQF | 296 |
| GLCPHCINV | 297 |
| GLENNVLYH | 298 |
| LHTDFEQVM | 299 |
| LLHTDFEQV | 300 |
| SESSFFNLI | 301 |
| SSHSGSFQI | 302 |
| TEADVQQWL | 303 |
| VQQWLTWCN | 304 |
| TAKSRVHPL | 305 |
| LLLIWFRPV | 306 |
| AVDTVLAKK | 307 |
| EPLVWIDCY | 308 |
| AITEVECFL | 309 |
| LLMWEAVTV | 310 |
| RARRELPRF | 311 |
| YLEKESIYY | 312 |
| NPKASLLSL | 313 |
| QVMLRWGVL | 314 |
| GILGFVFTL | 315 |
| SRYWAIRTR | 316 |
| NMLSTVLGV | 317 |
| LPFERATVM | 318 |
| LPFERATIM | 319 |
| LPFDKSTVM | 320 |
| LPFEKSTIM | 321 |
| LPFDKPTIM | 322 |
| LPFEKSTVM | 323 |
| CVNGSCFTV | 324 |
| LPFDRTTIM | 325 |
| KTGGPIYRR | 326 |
| LPFDRPTIM | 327 |
| GLDNHTILL | 328 |
| ILMWEAVTL | 329 |
| SITEVECFL | 330 |
| NLNESLIDL | 331 |
| RLDKVEAEV | 332 |
| VYDPLQPEL | 333 |
| TLKSFTVEK | 334 |
| HADQLTPTW | 335 |
| NATNVVIKV | 336 |
| VGYLQPRTF | 337 |
| YFQPRTFLL | 338 |
| FQFCNDPFL | 339 |
| ASVYAWNRK | 340 |
| GYLQPRTFL | 341 |
| YLQLRTFLL | 342 |
| YLQPRIFLL | 343 |
| AEVQIDRLI | 344 |
| ALNTLVKQL | 345 |
| CVADYSVLY | 346 |
| GSFCTQLNR | 347 |
| GVVFLHVTY | 348 |
| LLFNKVTLA | 349 |
| LLQYGSFCT | 350 |
| RLQSLQTYV | 351 |
| RVDFCGKGY | 352 |
| SVLNDILSR | 353 |
| VLNDILSRL | 354 |
| FVFKNIDGY | 355 |
| GTHWFVTQR | 356 |
| GTITSGWTF | 357 |
| KEIDRLNEV | 358 |
| LEPLVDLPI | 359 |
| LPPAYTNSF | 360 |
| NGVEGENCY | 361 |
| NQKLIANQF | 362 |
| RISNCVADY | 363 |
| RLFRKSNLK | 364 |
| TLDSKTQSL | 365 |
| TPINLVRDL | 366 |
| YFPLQSYGF | 367 |
| FQPTNGVGY | 368 |
| RFDNPVLPF | 369 |
| TSNQVAVLY | 370 |
| GLTVLPPLL | 371 |
| SIIAYTMSL | 372 |
| YLQPRTFLL | 373 |
| EPVLKGVKL | 374 |
| LTDEMIAQY | 375 |
| QYIKWPWYI | 376 |
| VLKGVKLHY | 377 |
| VTYVPAQEK | 378 |
| KCYGVSPTK | 379 |
| SPRRARSVA | 380 |
| YEQYIKWPW | 381 |
| AEIRASANL | 382 |
| GVYFASTEK | 383 |
| GVYYHKNNK | 384 |
| MIAQYTSAL | 385 |
| NSASFSTFK | 386 |
| NYNYLYRLF | 387 |
| ADAGFIKQY | 388 |
| ALDPLSETK | 389 |
| AYSNNSIAI | 390 |
| DAVRDPQTL | 391 |
| EILPVSMTK | 392 |
| ETKCTLKSF | 393 |
| EVFAQVKQI | 394 |
| EYVSQPFLM | 395 |
| FAMQMAYRF | 396 |
| FASVYAWNR | 397 |
| FERDISTEI | 398 |
| FPQSAPHGV | 399 |
| FTISVTTEI | 400 |
| FVIRGDEVR | 401 |
| FVSNGTHWF | 402 |
| GAAAYYVGY | 403 |
| GEVFNATRF | 404 |
| HLMSFPQSA | 405 |
| HVSGTNGTK | 406 |
| HWFVTQRNF | 407 |
| IAIPTNFTI | 408 |
| IANQFNSAI | 409 |
| INITRFQTL | 410 |
| IPFAMQMAY | 411 |
| IPTNFTISV | 412 |
| IYQTSNFRV | 413 |
| KIADYNYKL | 414 |
| KIYSKHTPI | 415 |
| KSNLKPFER | 416 |
| KVFRSSVLH | 417 |
| LGAENSVAY | 418 |
| LPFNDGVYF | 419 |
| LPLVSSQCV | 420 |
| LPPLLTDEM | 421 |
| NASVVNIQK | 422 |
| NATRFASVY | 423 |
| NSFTRGVYY | 424 |
| NSIAIPTNF | 425 |
| QELGKYEQY | 426 |
| QIAPGQTGK | 427 |
| QLTPTWRVY | 428 |
| QPTESIVRF | 429 |
| QTNSPRRAR | 430 |
| RSVASQSII | 431 |
| RVYSTGSNV | 432 |
| SANNCTFEY | 433 |
| SFKEELDKY | 434 |
| SVYAWNRKR | 435 |
| TLADAGFIK | 436 |
| VASQSIIAY | 437 |
| VFAQVKQIY | 438 |
| VFKNIDGYF | 439 |
| VGGNYNYLY | 440 |
| WFVTQRNFY | 441 |
| WTAGAAAYY | 442 |
| WTFGAGAAL | 443 |
| YGFQPTNGV | 444 |
| YNYLYRLFR | 445 |
| YQDVNCTEV | 446 |
| YYHKNNKSW | 447 |
| TQDLFLPFF | 448 |
| FVFLVLLPL | 449 |
| LVKNKCVNF | 450 |
| STQDLFLPF | 451 |
| NIADYNYKL | 452 |
| NYNYRYRLF | 453 |
| TIADYNYKL | 454 |
| LLYANSAHAL | 455 |
| SSGVVFGTWY | 456 |
| EYVHARWAAF | 457 |
| HTDLHPNNTY | 458 |
| AYLGAFLSVL | 459 |
| FVYTPSPYVF | 460 |
| AYSLLFPAPF | 461 |
| RPTERPRAPA | 462 |
| FTDALGIDEY | 463 |
| SALPTNADLY | 464 |
| LYPDAPPLRL | 465 |
| GFLIAYQPLL | 466 |
| FLVDAIVRVA | 467 |
| PHSVVNPFVK | 468 |
| MILIEGIFFV | 469 |
| TPRVTGGGAM | 470 |
| AYAQKIFKIL | 471 |
| YILEETSVML | 472 |
| RRKMMYMCYR | 473 |
| TYSQKIFKIL | 474 |
| RPPIFIRRLH | 475 |
| VEITPYKPTW | 476 |
| EENLLDFVRF | 477 |
| LLDFVRFMGV | 478 |
| PYLFWLAAIA | 479 |
| VMSNTLLSAW | 480 |
| QNGALAINTF | 481 |
| DTPLIPLTIF | 482 |
| ATVKTGNIKL | 483 |
| AERQGSPTPA | 484 |
| KPQGQRLIEV | 485 |
| QTDTYTLLGY | 486 |
| ILIEGIFFVS | 487 |
| HVFPHRFISF | 488 |
| RIRLVVPSAL | 489 |
| SLILIGITTL | 490 |
| KVEGEQHVIK | 491 |
| IAPYAGLIMI | 492 |
| KPAVGVYHIV | 493 |
| FSECNALGSY | 494 |
| STELNYNHLY | 495 |
| FLTEAIVHSV | 496 |
| YVLDLQPEAT | 497 |
| FACYDLCIVY | 498 |
| FAFSDLYVVY | 499 |
| FAFKDLCIVY | 500 |
| VAFTEIKIVY | 501 |
| YILDLQPETT | 502 |
| FVFADLRIVY | 503 |
| FAFSDLCIVY | 504 |
| ILTAFNSSHK | 505 |
| LTAPTGCIKK | 506 |
| TLKCLRYRFK | 507 |
| YYVHEGIRTY | 508 |
| YICEEASVTV | 509 |
| LLIRCINCOK | 510 |
| AVCDKCLKFY | 511 |
| CVYCKQQLLR | 512 |
| NPYAVCDKCL | 513 |
| RPRKLPQLCT | 514 |
| YAVCDKCLKF | 515 |
| QYNKPLCDLL | 516 |
| GIVCPICSQK | 517 |
| HGDTPTLHEY | 518 |
| HYNIVTFCCK | 519 |
| YMLDLQPETT | 520 |
| TIHDIILECV | 521 |
| HNIRGRWTGR | 522 |
| FAFRDLCIVY | 523 |
| QERPRKLPQL | 524 |
| RWTGRCMSCC | 525 |
| SSRTRRETQL | 526 |
| HPAATHTKAV | 527 |
| TLLQQYCLYL | 528 |
| GLCPHCINVG | 529 |
| HAKALKERMV | 530 |
| IDTCISATFR | 531 |
| LLHTDFEQVM | 532 |
| QSALKLAIYK | 533 |
| SALKLAIYKA | 534 |
| KLYQNPTTYI | 535 |
| RLYQNPTTYI | 536 |
| KFLPDLYDYK | 537 |
| VLRGFLILGK | 538 |
| GILGFVFTLT | 539 |
| RMVLASTTAK | 540 |
| SFSFGGFTFK | 541 |
| KNIDGYFKIY | 542 |
| CMTSCCSCLK | 543 |
| DSFKEELDKY | 544 |
| QPYRVVVLSF | 545 |
| SEPVLKGVKL | 546 |
| CALDPLSETK | 547 |
| EILDITPCSF | 548 |
| FTISVTTEIL | 549 |
| IGAEHVNNSY | 550 |
| ILPDPSKPSK | 551 |
| IYSKHTPINL | 552 |
| KLPDDFTGCV | 553 |
| KTSVDCTMYI | 554 |
| LSSTASALGK | 555 |
| STQDLFLPFF | 556 |
| SVLNDILSRL | 557 |
| TEKSNIIRGW | 558 |
| WIFGTTLDSK | 559 |
| YHLMSFPQSA | 560 |
| TQLNRALTGI | 561 |
| NESLIDLQEL | 562 |
| DGVYFASTEK | 563 |
| DSKVGGNYNY | 564 |
| EVFAQVKQIY | 565 |
| FDEDDSEPVL | 566 |
| FERDISTEIY | 567 |
| FEYVSQPFLM | 568 |
| FLPFFSNVTW | 569 |
| GVFVSNGTHW | 570 |
| GVYYPDKVFR | 571 |
| GYLQPRTFLL | 572 |
| HVTYVPAQEK | 573 |
| IHADQLTPTW | 574 |
| KFLPFQQFGR | 575 |
| KVGGNYNYLY | 576 |
| LPIGINITRF | 577 |
| NTSNQVAVLY | 578 |
| NVYADSFVIR | 579 |
| QIPFAMQMAY | 580 |
| QYIKWPWYIW | 581 |
| RASANLAATK | 582 |
| RFASVYAWNR | 583 |
| RVYSTGSNVF | 584 |
| SETKCTLKSF | 585 |
| STGSNVFQTR | 586 |
| SVASQSIIAY | 587 |
| SWMESEFRVY | 588 |
| TECSNLLLQY | 589 |
| TPCSFGGVSV | 590 |
| VFVSNGTHWF | 591 |
| VYSSANNCTF | 592 |
| YTNSFTRGVY | 593 |
| IYKTPPIKDF | 594 |
| LLTDEMIAQY | 595 |
| SYFIASFRLF | 596 |
| EITDTIDKFGK | 597 |
| LPEGMDPFAEK | 598 |
| ARLCDLPATPK | 599 |
| LQRGPQYSEHP | 600 |
| PSQEPMSIYVY | 601 |
| QEFFWDANDIY | 602 |
| QYDPVAALFFF | 603 |
| RLTVSGLAWTR | 604 |
| RNLVPMVATVQ | 605 |
| TPRVTGGGAMA | 606 |
| VFPTKDVALRH | 607 |
| VLCPKNMIIKP | 608 |
| YSEHPTFTSQY | 609 |
| YYTSAFVFPTK | 610 |
| QYTPDSTPCHR | 611 |
| RPHERNGFTVL | 612 |
| AKARAKKDELR | 613 |
| CYVLEETSVML | 614 |
| EFCRVLCCYVL | 615 |
| EQVTEDCNENP | 616 |
| ESLKTFEQVTE | 617 |
| FPKTTNGCSQA | 618 |
| GKSTHPMVTRS | 619 |
| KDELRRKMMYM | 620 |
| KNSAFPKTTNG | 621 |
| KQIKVRVDMVR | 622 |
| MAYAQKIFKIL | 623 |
| NIEFFTKNSAF | 624 |
| SVMKRRIEEIC | 625 |
| YPRNPTEQGNI | 626 |
| HPVGEADYFEY | 627 |
| QPRAPIRPIPT | 628 |
| SSCSSCPLSKI | 629 |
| EPLPQGOLTAY | 630 |
| AVYENPLSVEK | 631 |
| TEADVQQWLTW | 632 |
| FFAVGGDPLEM | 633 |
| IAVGLLLYCKA | 634 |
| TIAMELIRMIK | 635 |
| EILDITPCSFG | 636 |
| AQALNTLVKQL | 637 |
| FCNDPFLGVYY | 638 |
| FPQSAPHGVVF | 639 |
| KSWMESEFRVY | 640 |
| LQIPFAMQMAY | 641 |
| NYNYLYRLFRK | 642 |
| YEQYIKWPWYI | 643 |
| IPIGAGICASY | 644 |
| KPFERDISTEI | 645 |
| QEVFAQVKQIY | 646 |
| RFPNITNLCPF | 647 |
| YECDIPIGAGI | 648 |
| YENQKLIANQF | 649 |
| YYVGYLQPRTF | 650 |
| SFELLHAPATV | 651 |
| TYVPAQEKNFT | 652 |
| HPVGDADYFEY | 653 |
| HPVGQADYFEY | 654 |
| RVAGDSGFAAY | 655 |
| EFFWDANDIY | 656 |
| RPHERNGFTV | 657 |
| LPRRSGAAGA | 658 |
| ALQIPFAMQM | 659 |
| QELIRQGTDY | 660 |
| RARSVASQSI | 661 |
| RLITGRLQSL | 662 |
| TLATHGLAAV | 663 |
| TTDPSFLGRY | 664 |
| VENPHLMGWD | 665 |
| YPDKVFRSSV | 666 |
| AYAQKIFKI | 667 |
| CRVLCCYVL | 668 |
| CVETMCNEY | 669 |
| DEEDAIAAY | 670 |
| FRCPRRFCF | 671 |
| KLGGALQAK | 672 |
| MLNIPSINV | 673 |
| VMAPRTLIL | 674 |
| YILEETSVM | 675 |
| MLDLQPETT | 676 |
| CTELKLSDY | 677 |
| FEDLRVLSF | 678 |
| ALSKGVHFV | 679 |
| ALWEIQQVV | 680 |
| DTDFVNEFY | 681 |
| FIAGLIAIV | 682 |
| FTSDYYQLY | 683 |
| KQIYKTPPI | 684 |
| KTFPPTEPK | 685 |
| LLLDRLNQL | 686 |
| LLYDANYFL | 687 |
| MLAKALRKV | 688 |
| MQLFFSYFA | 689 |
| PTDNYITTY | 690 |
| SPRWYFYYL | 691 |
| VYFLQSINF | 692 |
| VYIGDPAQL | 693 |
| FPTKDVAL | 694 |
| NEGVKAAW | 695 |
| DYNFVKQLF | 696 |
| TYPVLEEMF | 697 |
| RYSIFFDYM | 698 |
| TYSAGIVQI | 699 |
| VYALPLKML | 700 |
| GPISGHVLK | 701 |

In some embodiments, the immunogenic peptide is inserted into a complementarity determining region (CDR) of the antibody or antigen binding fragment thereof. In some embodiments, insertion of the immunogenic peptide comprises removal of CDR sequence. In some embodiments, at least one CDR of the antibody or antigen binding fragment thereof is replaced with the immunogenic peptide. In some embodiments, the CDR is the whole CDR. In some embodiments, the CDR is at least a portion of the CDR. In some embodiments, CDR sequence comprises sequence of at least a portion of the CDR. In some embodiments, a portion is at least 4 amino acids. In some embodiments, a portion is at least 5 amino acids. In some embodiments, a portion is at least 6 amino acids. In some embodiments, a portion is at least 7 amino acids. In some embodiments, a portion is at least 8 amino acids. In some embodiments, a portion is at least 9 amino acids. In some embodiments, insertion of the immunogenic peptide comprises removal of the CDR. In some embodiments, insertion of the immunogenic peptide comprises removal of at least a portion of the CDR.

In some embodiments, replacement of the CDR or a portion of the CDR also comprises replacement of at least one amino acid flanking the CDR. In some embodiments, flanking is N-terminal to the CDR. In some embodiments, flanking is C-terminal to the CDR. In some embodiments, at least one amino acid is at least the 1, 2, 3, 4 or 5 amino acids directly flanking the CDR. Each possibility represents a separate embodiment of the invention. In some embodiments, at least one amino acid is 4-5 amino acids. In some embodiments, at least one amino acid is 4 amino acids. In some embodiments, at least one amino acid is 5 amino acids. In some embodiments, the flanking region is not more than 5, 6, 7, 8, 9 or 10 amino acids. Each possibility represents a separate embodiment of the invention. In some embodiments, the flanking region is not more than 5 amino acids. In some embodiments, the flanking region is not more than 10 amino acids. In some embodiments, a flanking region comprises a stem of the CDR loop. It will be understood by a skilled artisan that in order to preserve antibody structure and to produce as little perturbation as possible it may be necessary to also replace a portion of the CDR flanking regions along with the CDR itself (or a portion thereof).

In some embodiments, the CDRH1 of TMab4 comprises or consists of amino acid 26-33 of SEQ ID NO: 1021. In some embodiments, the CDRH2 of TMab4 comprises or consists of amino acid 51-58 of SEQ ID NO: 1021. In some embodiments, the CDRH3 of TMab4 comprises or consists of amino acid 97-109 of SEQ ID NO: 1021. In some embodiments, the CDRL1 of TMab4 comprises or consists of amino acid of 27-38SEQ ID NO: 1022. In some embodiments, the CDRL2 of TMab4 comprises or consists of amino acid 56-58 of SEQ ID NO: 1022. In some embodiments, the CDRL3 of TMab4 comprises or consists of amino acid 95-103 of SEQ ID NO: 1022. In some embodiments, the CDRH1 of 3E10 comprises or consists of amino acid 26-33 of SEQ ID NO: 1023. In some embodiments, the CDRH2 of 3E10 comprises or consists of amino acid 51-58 of SEQ ID NO: 1023. In some embodiments, the CDRH3 of 3E10 comprises or consists of amino acid 97-105 of SEQ ID NO: 1023. In some embodiments, the CDRL1 of 3E10 comprises or consists of amino acid 27-36 of SEQ ID NO: 1024. In some embodiments, the CDRL2 of 3E10 comprises or consists of amino acid 54-56 of SEQ ID NO: 1024. In some embodiments, the CDRL3 of 3E10 comprises or consists of amino acid 93-101 of SEQ ID NO: 1024. In some embodiments, the CDRH1 of 71F12 comprises or consists of amino acid 26-33 of SEQ ID NO: 1026. In some embodiments, the CDRH2 of 71F12 comprises or consists of amino acid 51-57 of SEQ ID NO: 1026. In some embodiments, the CDRH3 of 71F12 comprises or consists of amino acid 96-105 of SEQ ID NO: 1026. In some embodiments, the CDRL1 of 71F12 comprises or consists of amino acid 26-34 of SEQ ID NO: 1027. In some embodiments, the CDRL2 of 71F12 comprises or consists of amino acid 52-54 of SEQ ID NO: 1027. In some embodiments, the CDRL3 of 71F12 comprises or consists of amino acid 91-100 of SEQ ID NO: 1027.

In some embodiments, the antigen binding molecule comprises a cell penetration sequence. In some embodiments, the cell penetration sequence is a cell penetration domain. In some embodiments, the cell penetration sequence is a cell penetration peptide. In some embodiments, the cell penetration sequence targets the molecule to the inside of the cell. In some embodiments, the cell penetration sequence delivers the molecule to the inside of the cell. In some embodiments, the cell penetration sequence enables entrance of the molecule to the inside of the cell. In some embodiments, the inside of the cell is an endosome. In some embodiments, the inside of the cell is the cytoplasm. In some embodiments, delivery to the cytoplasm comprises exit from an endosome. In some embodiments, delivery to the cytoplasm comprises escape from an endosome. In some embodiments, an endosome is the endosomal pathway. In some embodiments, the cell penetration sequence is a peptide transduction domain (PTD). In some embodiments, the cell penetration sequence is a cell penetrating peptide (CPP). In some embodiments, the cell penetration sequence is an endosomal escape domain (EED). Peptide sequences that allow entrance into the cytoplasm and in particular escape from the endosomal pathway after endocytosis are well known in the art and any such peptide sequence may be employed.

It is known in the art that the CDRL1 and CDRL3 of Tmab4 are both play a role in antibody penetration (see Kim et al., “Endosomal acidic pH-induced conformational changes of a cytosol-penetrating antibody mediate endosomal escape”, J Control Release, 2016, 10; 235:165-175 and Choi et al., “A general strategy for generating intact, full-length IgG antibodies that penetrate into the cytosol of living cells”, Mabs, 2014; 6(6):1402-14, herein incorporated by reference in their entirety. In some embodiments, the cell penetration sequence comprises the CDRL1 of Tmab4. In some embodiments, the cell penetration sequence consists of the CDRL1 of Tmab4. In some embodiments, the cell penetration sequence comprises the CDRL3 of Tmab4. In some embodiments, the cell penetration sequence consists of the CDRL3 of Tmab4. Methods of grafting the cell penetration sequence into other antibodies are known in the art, such as for example, the method provided in Choi et al.

In some embodiments, the antigen binding molecule is a bi-specific antibody. In some embodiments, the antigen binding molecule is a bi-specific antibody fragment of an antibody. In some embodiments, the antigen binding molecule comprises a plurality of antigen binding regions. In some embodiments, the antigen binding molecule comprises at least two antigen binding regions. In some embodiments, the antigen binding molecule comprises two antigen binding regions. In some embodiments, at least one of the antigen binding regions is the antigen binding region that is capable of binding to a target cell. In some embodiments, at least one of the antigen binding regions is mutated to comprises the immunogenic peptide. In some embodiments, at least one of the antigen binding regions is mutated to comprises the cell penetration sequence. In some embodiments, at least one of the antigen binding regions is mutated to comprises the immunogenic peptide, the cell penetration sequence or both. In some embodiments, the mutation is mutation of a complementarity determining region (CDR). In some embodiments, a CDR of an antigen binding region is mutated. In some embodiments, mutated is replaced. In some embodiments, replaced is replaced with the immunogenic peptide. In some embodiments, replaced is replaced with the cell penetration sequence.

In some embodiments, the CDR is an inert CDR. In some embodiments, an inert CDR does not contribute to binding to a target antigen. In some embodiments, binding is binding of the antigen binding region comprising the CDR to the target antigen. In some embodiments, an inert CDR comprises little or no contribution to binding. In some embodiments, an inert CDR comprises two or fewer amino acids that contact the target antigen. In some embodiments, an inert CDR comprises fewer than 2 amino acids that contact the target antigen. In some embodiments, an inert CDR comprises 2, 1 or 0 amino acids that contact the target antigen. In some embodiments, an inert CDR comprises 2 amino acid that contacts the target antigen. In some embodiments, an inert CDR comprises 1 amino acid that contacts the target antigen. In some embodiments, an inert CDR does not comprise an amino acid that contacts the target antigen. In some embodiments, an inert CDR does not contact the target antigen. In some embodiments, contact comprises a distance of not more than 3, 5, 7, 9 or 10 angstroms. Each possibility represents a separate embodiment of the invention. In some embodiments, contact comprises a distance of not more than 5 angstroms.

In some embodiments, the distance is between the amino acid of the CDR and an amino acid of the target antigen. In some embodiments, the distant is the distance present when the antigen binding domain is bound to the antigen. In some embodiments, the distance is the distance during crystallography studies of the binding.

In some embodiments, mutation or replacement of an inert CDR does not diminish binding. In some embodiments, not diminishing binding is not significantly diminishing binding. In some embodiments, mutation or replacement of an inert CDR does not abrogate binding. In some embodiments, a significant diminishment is a reduction in binding of more than 5, 10, 15, 20, 25, 30, 35, 40, 45, 50, 60, 70, 75, 80, 90, 95 or 99%. Each possibility represents a separate embodiment of the invention. In some embodiments, a significant diminishment is a reduction in binding of more than 10%. In some embodiments, a significant diminishment is a reduction in binding of more than 20%. In some embodiments, mutation or replacement of an inert CDR does not reduce binding by 100%.

In some embodiments, mutation or replacement of an inert CDR does not diminish cell penetrance. In some embodiments, not diminishing cell penetrance is not significantly diminishing cell penetrance. In some embodiments, mutation or replacement of an inert CDR does not abrogate cell penetrance. In some embodiments, a significant diminishment is a reduction in penetrance of more than 5, 10, 15, 20, 25, 30, 35, 40, 45, 50, 60, 70, 75, 80, 90, 95 or 99%. Each possibility represents a separate embodiment of the invention. In some embodiments, a significant diminishment is a reduction in penetrance of more than 10%. In some embodiments, a significant diminishment is a reduction in penetrance of more than 20%. In some embodiments, mutation or replacement of an inert CDR does not reduce penetrance by 100%. In some embodiments, mutation or replacement of an inert CDR does not reduce penetrance to the level of a control antibody. In some embodiments, a control antibody is an antibody that does not enter a cell. Antibodies that do not enter cells are well known in the art, and include for example adalimumab and muromonab. In some embodiments, not diminishing cell penetrance comprises retaining penetrance that is substantially equal to the binding of the antibody or antigen binding fragment thereof devoid of or without the immunogenic peptide.

In some embodiments, insertion of the immunogenic peptide and removal of CDR sequence produces no change in the conformation of the antibody or antigen binding fragment thereof. In some embodiments, insertion of the immunogenic peptide and removal of CDR sequence produces minimal change in the conformation of the antibody or antigen binding fragment thereof. In some embodiments, conformation is overall conformation. In some embodiments, conformation is 3D structure. In some embodiments, conformation is tertiary structure. In some embodiments, change is perturbation. In some embodiments, minimal change is without a loss of a bond. In some embodiments, minimal change is a change of less than 5, 10, 15, 20, 25, 30, 35, 40, 45 or 50%. Each possibility represents a separate embodiment of the invention. In some embodiments, minimal change comprises binding of a target antigen at an equivalent affinity to the antibody or antigen binding fragment thereof devoid or without the immunogenic peptide. In some embodiments, equivalent is with no reduction in affinity. In some embodiments, equivalent is with a reduction in affinity of not more than 5, 10, 15, 20, 25, 30, 35, 40, 45 or 50%. Each possibility represents a separate embodiment of the invention. In some embodiments, equivalent is with a reduction in affinity of not more than 10%. In some embodiments, equivalent is with a reduction in affinity of not more than 20%

In some embodiments, the mutated CDR is in the antigen binding region capable of binding the target cell. In some embodiments, the mutated CDR is not in the antigen binding region capable of binding the target cell. In some embodiments, the mutated CDR is in an antigen binding region other than the antigen binding region capable of binding the target cell. In some embodiments, the immunogenic peptide replaces a CDR in the antigen binding region capable of binding the target cell. In some embodiments, the cell penetrating sequence replaces a CDR in an antigen binding region that is not the antigen binding region that binds the target cell. In some embodiments, the immunogenic peptide and the cell penetration sequence are in the same antigen binding region. In some embodiments, the immunogenic peptide and the cell penetration sequence are in different antigen binding regions.

In some embodiments, the target cell is a dendritic cell, the dendritic cell antigen is CD40, the antigen binding region capable of binding to CD40 is Fab516 and light chain CDR L2 is replaced with said cell penetrating sequence, said immunogenic peptide or both.

In some embodiments, the target cell is a B cell, the B cell antigen is CD20, the antigen binding region capable of binding to CD20 is Arzerra and at least one of heavy chain CDR H1, light chain CDR L1 and light chain CDR L2 is replaced with said cell penetrating sequence, said immunogenic peptide or both. In some embodiments, replaced is mutated to include the peptide or sequence. In some embodiments, heavy chain CDR H1 is replaced. In some embodiments, the light chain CDR L1 is replaced. In some embodiments, the light chain CDR L2 is replaced.

In some embodiments, the target cell is a cancer cell, the cancer cell antigen is PD-L1, the antigen binding region capable of binding to PD-L1 is Durvalumab and at least one of heavy chain CDR H1, and light chain CDR L2 is replaced with said cell penetrating sequence, said immunogenic peptide or both. In some embodiments, replaced is mutated to include the peptide or sequence. In some embodiments, heavy chain CDR H1 is replaced. In some embodiments, the light chain CDR L2 is replaced.

In some embodiments, the antibody is TMab4 and the immunogenic peptide is inserted into any one of CDRH1, CDRH2, CDRH3 and CDRL3. In some embodiments, the antibody is TMab4 and the immunogenic peptide is inserted into CDRH1. In some embodiments, the antibody is TMab4 and the immunogenic peptide is inserted into CDRH2. In some embodiments, the antibody is TMab4 and the immunogenic peptide is inserted into CDRH3. In some embodiments, the antibody is TMab4 and the immunogenic peptide is inserted into CDRL3. In some embodiments, SEQ ID NO: 1 is inserted into CDRH1 of TMab4. In some embodiments, SEQ ID NO: 2 is inserted into CDRH1 of TMab4. In some embodiments, SEQ ID NO: 3 is inserted into CDRH1 of TMab4. In some embodiments, SEQ ID NO: 4 is inserted into CDRH1 of TMab4. In some embodiments, SEQ ID NO: 5 is inserted into CDRH1 of TMab4. In some embodiments, SEQ ID NO: 1 is inserted into CDRH2 of TMab4. In some embodiments, SEQ ID NO: 2 is inserted into CDRH2 of TMab4. In some embodiments, SEQ ID NO: 3 is inserted into CDRH2 of TMab4. In some embodiments, SEQ ID NO: 4 is inserted into CDRH2 of TMab4. In some embodiments, SEQ ID NO: 5 is inserted into CDRH2 of TMab4. In some embodiments, SEQ ID NO: 1 is inserted into CDRH3 of TMab4. In some embodiments, SEQ ID NO: 2 is inserted into CDRH3 of TMab4. In some embodiments, SEQ ID NO: 3 is inserted into CDRH3 of TMab4. In some embodiments, SEQ ID NO: 4 is inserted into CDRH3 of TMab4. In some embodiments, SEQ ID NO: 5 is inserted into CDRH3 of TMab4. In some embodiments, SEQ ID NO: 1 is inserted into CDRL3 of TMab4. In some embodiments, SEQ ID NO: 2 is inserted into CDRL3 of TMab4. In some embodiments, SEQ ID NO: 3 is inserted into CDRL3 of TMab4. In some embodiments, SEQ ID NO: 4 is inserted into CDRL3 of TMab4. In some embodiments, SEQ ID NO: 5 is inserted into CDRL3 of TMab4. In some embodiments, SEQ ID NO: 6 is inserted into CDRH1 of TMab4. In some embodiments, SEQ ID NO: 7 is inserted into CDRH23 of TMab4. In some embodiments, SEQ ID NO: 8 is inserted in place of amino acids 14-22 of the light chain of TMab4. In some embodiments, into CDRH1 comprises replacing amino acids 25-33 of the heavy chain. In some embodiments, into CDRH1 comprises replacing amino acids 26-33 of the heavy chain. In some embodiments, into CDRH1 comprises replacing amino acids 26-32 of the heavy chain. In some embodiments, into CDRH1 comprises replacing amino acids 27-33 of the heavy chain. In some embodiments, into CDRH1 comprises replacing amino acids 28-33 of the heavy chain. In some embodiments, into CDRH1 comprises replacing amino acids 22-30 of the heavy chain. In some embodiments, into CDRH1 comprises replacing amino acids 22-29 of the heavy chain. In some embodiments, into CDRH1 comprises replacing amino acids 26-31 of the heavy chain. In some embodiments, into CDRH1 comprises replacing amino acids 23-32 of the heavy chain. In some embodiments, into CDRH1 comprises replacing amino acids 23-31 of the heavy chain. In some embodiments, into CDRH1 comprises replacing amino acids 28-35 of the heavy chain. In some embodiments, into CDRH3 comprises replacing amino acids 100-108 of the heavy chain. In some embodiments, into CDRH3 comprises replacing amino acids 99-106 of the heavy chain. In some embodiments, into CDRH3 comprises replacing amino acids 99-105 of the heavy chain. In some embodiments, into CDRH3 comprises replacing amino acids 100-106 of the heavy chain. In some embodiments, into CDRH3 comprises replacing amino acids 100-105 of the heavy chain. In some embodiments, into CDRH3 comprises replacing amino acids 100-104 of the heavy chain. In some embodiments, into CDRH3 comprises replacing amino acids 99-107 of the heavy chain. In some embodiments, into CDRH2 comprises replacing amino acids 52-59 of the heavy chain. In some embodiments, into CDRH2 comprises replacing amino acids 52-60 of the heavy chain. In some embodiments, into CDRL3 comprises replacing amino acids 97-103 of the light chain. In some embodiments, into CDRL3 comprises replacing amino acids 98-103 of the light chain. In some embodiments, into CDRL3 comprises replacing amino acids 96-104 of the light chain. In some embodiments, into CDRL3 comprises replacing amino acids 98-104 of the light chain.

In some embodiments, the antibody is 3E10 and the immunogenic peptide is inserted into any one of CDRL1 and CDRL2. In some embodiments, the antibody is 3E10 and the immunogenic peptide is inserted into CDRL1. In some embodiments, the antibody is 3E10 and the immunogenic peptide is inserted into CDRL2. In some embodiments, SEQ ID NO: 1 is inserted into CDRL1 of 3E10. In some embodiments, SEQ ID NO: 2 is inserted into CDRL1 of 3E10. In some embodiments, SEQ ID NO: 3 is inserted into CDRL1 of 3E10. In some embodiments, SEQ ID NO: 4 is inserted into CDRL1 of 3E10. In some embodiments, SEQ ID NO: 5 is inserted into CDRL1 of 3E10. In some embodiments, SEQ ID NO: 1 is inserted into CDRL2 of 3E10. In some embodiments, SEQ ID NO: 2 is inserted into CDRL2 of 3E10. In some embodiments, SEQ ID NO: 3 is inserted into CDRL2 of 3E10. In some embodiments, SEQ ID NO: 4 is inserted into CDRL2 of 3E10. In some embodiments, SEQ ID NO: 5 is inserted into CDRL2 of 3E10. In some embodiments, SEQ ID NO: 6 is inserted into CDRL1 of 3E10. In some embodiments, SEQ ID NO: 9 is inserted into CDRL2 of 3E10. In some embodiments, into CDRL1 comprises replacing amino acids 27-35 of the light chain. In some embodiments, into CDRL1 comprises replacing amino acids 28-36 of the light chain. In some embodiments, into CDRL2 comprises replacing amino acids 50-58 of the light chain.

In some embodiments, the antibody is 71F12 and the immunogenic peptide is inserted into CDRL1. In some embodiments, SEQ ID NO: 1 is inserted into CDRL1 of 71F12. In some embodiments, SEQ ID NO: 2 is inserted into CDRL1 of 71F12. In some embodiments, SEQ ID NO: 3 is inserted into CDRL1 of 71F12. In some embodiments, SEQ ID NO: 4 is inserted into CDRL1 of 71F12. In some embodiments, SEQ ID NO: 5 is inserted into CDRL1 of 71F12. In some embodiments, into CDRL1 comprises replacing amino acids 28-36 of the light chain. In some embodiments, into CDRL1 comprises replacing amino acids 26-34 of the light chain. In some embodiments, into CDRL2 comprises replacing amino acids 50-58 of the light chain.

In some embodiments, the antibody is a commercially available antibody. In some embodiments, the antibody penetrates into a bound cell at a level comparable to any one of TMab4, 3E10 and 71F12. In some embodiments, the antibody penetrates into a bound cell at a level comparable to TMab4. In some embodiments, the antibody penetrates into a bound cell at a level comparable to 3E10. In some embodiments, the antibody penetrates into a bound cell at a level comparable to 71F12. In some embodiments, comparable is with a penetrance that is at least 50, 55, 60, 65, 70, 75, 80, 85, 90, 92, 95, 97, 99 or 100% of the original antibody. Each possibility represents a separate embodiment of the invention. In some embodiments, comparable is with a penetrance that is at least 80% of the original antibody. In some embodiments, comparable is with a penetrance that is at least 90% of the original antibody.

In some embodiments, the antigen binding region and the immunogenic peptide are part of the same amino acid chain. In some embodiments, the antigen binding region and the cell penetration sequence are part of the same amino acid chain. In some embodiments, the immunogenic peptide and the cell penetration sequence are part of the same amino acid chain. In some embodiments, the antigen binding molecule of the invention is a single fusion protein. In some embodiments, the antigen binding molecule of the invention is a single amino acid chain.

In some embodiments, the antigen binding region and the immunogenic peptide are separated by a linker. In some embodiments, the antigen binding region and the cell penetrating sequence are separated by a linker. In some embodiments, the immunogenic peptide and the cell penetrating sequence are separated by a linker. In some embodiments, the linker is not a chemical linker. In some embodiments, the linker is not an artificial linker. In some embodiments, the linker is a peptide linker. In some embodiments, the linker is an amino acid linker. In some embodiments, the linker comprises or consists of at least 1, 2, 3, 4 or 5 amino acids. Each possibility represents a separate embodiment of the invention. In some embodiments, the linker comprises or consists of at most 1, 2, 3, 4, 5, 6, 7, 8, 9 or 10 amino acids. Each possibility represents a separate embodiment of the invention. In some embodiments, the linker comprises or consists of 1, 2, 3, 4, 5, 6, 7, 8, 9 or 10 amino acids. Each possibility represents a separate embodiment of the invention.

In some embodiments, the antibody is selected from antibodies T1-T19, T1_30-T1_35, T1_39-T1_45 and T1_47. In some embodiments, the antibody or antigen binding fragment thereof comprises a light chain variable region comprising SEQ ID NO: 1022 and a heavy chain variable region comprising a sequence selected from SEQ ID NO: 1028-1040, 1043-1045, 1047-1055, and 1058-1059 or analogs or homologs comprising at least 85% sequence identity. In some embodiments, an analog or homolog comprises the immunogenic peptide. In some embodiments, an analog or homolog is capable of binding cells. In some embodiments, an analog or homolog is delivered to the cytosol upon cell binding. In some embodiments, reaching the cytosol comprises displaying the immunogenic peptide on the surface of the bound cell. In some embodiments, the antibody or antigen binding fragment thereof comprises a light chain variable region comprising SEQ ID NO: 1022 and a heavy chain variable region comprising SEQ ID NO: 1028 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1029 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1030 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1031 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1032 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1033 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1034 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1035 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1036 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1037 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1038 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1039 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1040 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1043 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1044 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1045 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1047 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1048 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1049 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1050 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1051 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1052 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1053 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1054 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1055 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1058 or analogs or homologs comprising at least 85% sequence identity. In some embodiments, the antibody or antigen binding fragment thereof comprises a light chain variable region comprising SEQ ID NO: 1022 and a heavy chain variable region comprising SEQ ID NO: 1059 or analogs or homologs comprising at least 85% sequence identity.

In some embodiments, the antibody or antigen binding fragment thereof comprises a heavy chain variable region comprising SEQ ID NO: 1021 and a light chain variable region comprising a sequence selected from SEQ ID NO: 1041-1042, 1046, and 1056-1057 or analogs or homologs comprising at least 85% sequence identity. In some embodiments, the antibody or antigen binding fragment thereof comprises a heavy chain variable region comprising SEQ ID NO: 1021 and a light chain variable region comprising SEQ ID NO: 1041 or analogs or homologs comprising at least 85% sequence identity. In some embodiments, the antibody or antigen binding fragment thereof comprises a heavy chain variable region comprising SEQ ID NO: 1021 and a light chain variable region comprising SEQ ID NO: 1042 or analogs or homologs comprising at least 85% sequence identity. In some embodiments, the antibody or antigen binding fragment thereof comprises a heavy chain variable region comprising SEQ ID NO: 1021 and a light chain variable region comprising SEQ ID NO: 1046 or analogs or homologs comprising at least 85% sequence identity. In some embodiments, the antibody or antigen binding fragment thereof comprises a heavy chain variable region comprising SEQ ID NO: 1021 and a light chain variable region comprising SEQ ID NO: 1056 or analogs or homologs comprising at least 85% sequence identity. In some embodiments, the antibody or antigen binding fragment thereof comprises a heavy chain variable region comprising SEQ ID NO: 1021 and a light chain variable region comprising SEQ ID NO: 1057 or analogs or homologs comprising at least 85% sequence identity.

In some embodiments, the antibody is selected from antibodies T2_6, T2_11-T2_13, T2_20 and T2_23. In some embodiments, the antibody or antigen binding fragment thereof comprises a heavy chain variable region comprising SEQ ID NO: 1023 and a light chain variable region comprising a sequence selected from SEQ ID NO: 1060-1065 or analogs or homologs comprising at least 85% sequence identity. In some embodiments, the antibody or antigen binding fragment thereof comprises a heavy chain variable region comprising SEQ ID NO: 1023 and a light chain variable region comprising SEQ ID NO: 1060 or analogs or homologs comprising at least 85% sequence identity. In some embodiments, the antibody or antigen binding fragment thereof comprises a heavy chain variable region comprising SEQ ID NO: 1023 and a light chain variable region comprising SEQ ID NO: 1061 or analogs or homologs comprising at least 85% sequence identity. In some embodiments, the antibody or antigen binding fragment thereof comprises a heavy chain variable region comprising SEQ ID NO: 1023 and a light chain variable region comprising SEQ ID NO: 1062 or analogs or homologs comprising at least 85% sequence identity. In some embodiments, the antibody or antigen binding fragment thereof comprises a heavy chain variable region comprising SEQ ID NO: 1023 and a light chain variable region comprising SEQ ID NO: 1063 or analogs or homologs comprising at least 85% sequence identity. In some embodiments, the antibody or antigen binding fragment thereof comprises a heavy chain variable region comprising SEQ ID NO: 1023 and a light chain variable region comprising SEQ ID NO: 1064 or analogs or homologs comprising at least 85% sequence identity. In some embodiments, the antibody or antigen binding fragment thereof comprises a heavy chain variable region comprising SEQ ID NO: 1023 and a light chain variable region comprising SEQ ID NO: 1065 or analogs or homologs comprising at least 85% sequence identity.

In some embodiments, the antibody is selected from antibodies T4_1 and T4_3. In some embodiments, the antibody or antigen binding fragment thereof comprises a heavy chain variable region comprising SEQ ID NO: 1026 and a light chain variable region comprising SEQ ID NO: 1066 or analogs or homologs comprising at least 85% sequence identity. In some embodiments, the antibody or antigen binding fragment thereof comprises a heavy chain variable region comprising SEQ ID NO: 1027 and a light chain variable region comprising SEQ ID NO: 1067 or analogs or homologs comprising at least 85% sequence identity.

In some embodiments, at least 85% identity is at least 90, 92, 95, 97, 99 or 100% identity. Each possibility represents a separate embodiment of the invention. It will be understood by a skilled artisan that while certain regions in the antibodies are required for function, such as the antigen binding CDRs, the immunogenic peptide and the cell penetrating moiety, other regions may bear alterations without altering function. Other inert CDRs are such a region as are many inter-CDR sequences as well as sequences within the constant region of an antibody. Analogs and homologs that retain function but contain alterations with these other regions are also encompassed within the invention.

In some embodiments, the antigen biding region is a single-chain antibody. In some embodiments, the antigen binding molecule of the invention comprises a first single-chain antibody and a second single chain antibody. In some embodiments, the first single-chain antibody is capable of binding the antigen of the target cell. In some embodiments, the second single-chain antibody is not functional. In some embodiments, the second single-chain antibody comprises the cell penetrating sequence. In some embodiments, the first single-chain antibody comprises the immunogenic peptide. In some embodiments, the second single-chain antibody comprises the immunogenic peptide. In some embodiments, the first single-chain antibody comprises an inert CDR replaced with the immunogenic peptide. In some embodiments, the first single-chain antibody comprises an inert CDR replaced with the cell penetrating sequence. In some embodiments, the first single-chain antibody and the second single-chain antibody are separated by a linker. In some embodiments, the linker is a peptide linker or an amino acid linker. In some embodiments, the peptide linker is an amino acid linker. In some embodiments, a peptide linker is a peptide bond.

In some embodiments, the antigen biding molecule is an antibody. In some embodiments, the antigen biding molecule comprises a first heavy chain and a first light chain. In some embodiments, the first antigen binding region comprises a first heavy chain and a first light chain. In some embodiments, the first heavy chain and the first light chain are capable of binding the antigen on a target cell. In some embodiments, a CDR of the first heavy chain is inert. In some embodiments, a CDR of the first light chain is inert. In some embodiments, an inert CDR of the first heavy chain is replaced with the immunogenic peptide. In some embodiments, an inert CDR of the first light chain is replaced with the immunogenic peptide. In some embodiments, an inert CDR of the first heavy chain is replaced with the cell penetration sequence. In some embodiments, an inert CDR of the first light chain is replaced with the cell penetration sequence.

In some embodiments, the antigen biding molecule comprises a second heavy chain. In some embodiments, the antigen biding molecule comprises a second light chain. In some embodiments, the antigen biding molecule comprises a second heavy chain and a second light chain. In some embodiments, the second antigen binding region comprises a second heavy chain. In some embodiments, the second antigen binding region comprises a second light chain. In some embodiments, the second antigen binding region comprises a second heavy chain and a second light chain. In some embodiments, a CDR of the second heavy chain is inert. In some embodiments, a CDR of the second light chain is inert. In some embodiments, the second heavy chain comprises the immunogenic peptide. In some embodiments, the second light chain comprises the immunogenic peptide. In some embodiments, the second heavy chain comprises the cell penetrating sequence. In some embodiments, the second light chain comprises the cell penetrating sequence. In some embodiments, a CDR of the second light chain is replaced. In some embodiments, a CDR of the second heavy chain is replaced. In some embodiments, an inert CDR of the second heavy chain is replaced with the immunogenic peptide. In some embodiments, an inert CDR of the second light chain is replaced with the immunogenic peptide. In some embodiments, an inert CDR of the second heavy chain is replaced with the cell penetration sequence. In some embodiments, an inert CDR of the second light chain is replaced with the cell penetration sequence.

In some embodiments, the antigen binding molecule is a dual-function antigen binding molecule. In some embodiments, the composition comprises a dual-function antigen binding molecule. In some embodiments, the dual-function antigen binding molecule comprises an antibody or antigen binding fragment of the invention. In some embodiments, the dual-function antigen binding molecule is a bi-specific antibody. In some embodiments, the dual function antigen binding molecule comprises a first antibody or antigen binding molecule and a second antibody or antigen binding molecule. In some embodiments, the first antibody is a first heavy chain and a first light chain and the second antibody and a second heavy chain and a second light chain. In some embodiments, the heavy chain and light chain are hybridized between the CH1 domain of the heavy chain and the CL domain of the light chain. In some embodiments, hybridized is bonded. In some embodiments, hybridized comprises disulfide bonds. In some embodiments, the second antibody or antigen binding fragment thereof is capable of binding an antigen overexpressed on a target cell. In some embodiments, the target cell is a cancer cell. In some embodiments, the antigen is a cancer antigen.

Examples of cancer antigens include but are not limited to epidermal growth factor (EGFR), Receptor tyrosine-protein kinase erbB2 (HER2), Nectin cell adhesion molecule 4 (NECTIN-4), Tumor-associated calcium signal transducer 2 (TROP-2/TACSTD2), Tissue Factor (TF/F3), B-cell maturation antigen (BCMA/TNFRSF17), Programmed death-ligand 1 (PDL-1), T cell immunoreceptor with Ig and ITIM domains (TIGIT), Epithelial cell adhesion molecule (EpCAM), TNF receptor superfamily member 8 (CD3/TNFRSF8), B-lymphocyte antigen CD19 (CD19), cluster of differentiation-22 (CD22), Siglec-3 (CD33), cluster of differentiation 38 (CD38), Cluster of differentiation 79 (CD79), Lymphocyte-activation gene 3 (LAG-3), C-C MotifChemokine Receptor 4 (CCR4), vascular endothelial growth factor receptor 2 (VEGFR2/KDR), Folate receptor 1 (FOLR1), CAMPATH-1 antigen (CD52), platelet-derived growth factor receptor A (PDGFRα), disialoganglio side GD2, mono sialodihexo sylganglio side (GM3), insulin-like growth factor 1 (IGF-1) receptor (IGF1R), SLAM family member 7 (SLAMF7), and cytotoxic T-lymphocyte-associated protein 4 (CTLA-4). Antibodies that bind to these cancer antigens are well known in the art and any such antibody can be used as the targeting module of the invention. In some embodiments, the cancer antigen targeted by the targeting molecule is selected from HER2, EGFR, EpCAM, BCMA, CD33, CD38, CTLA, LAG-3, and PD-L1. Examples of antibodies that can be used for the targeting moiety are provided in Table 4.

TABLE 4
targeting moiety antibodies

| International Non-Proprietary Name | Target |
|---|---|
| Omburtamab | B7-H3 (CD276) |
| Belantamab mafodotin (belantamab mafodotin-blmf) | BCMA |
| Teclistamab | BCMA, CD3 |
| Mogamulizumab (mogamulizumab-kpkc) | CCR4 |
| Loncastuximab tesirine | CD19 |
| Tafasitamab (tafasitamab-cxix) | CD19 |
| Blinatumomab | CD19, CD3 |
| Ibritumomab tiuxetan | CD20 |
| Obinutuzumab | CD20 |
| Ofatumumab | CD20 |
| Ripertamab | CD20 |
| Rituximab | CD20 |
| Tositumomab-I131 | CD20 |
| Mosunetuzumab | CD20, CD3 |
| Glofitamab | CD20, CD3e |
| Inotuzumab ozogamicin | CD22 |
| Moxetumomab pasudotox | CD22 |
| (moxetumomab pasudotox-tdfk) | |
| Brentuximab vedotin | CD30 |
| Gemtuzumab ozogamicin | CD33 |
| Daratumumab | CD38 |
| Isatuximab (isatuximab-irfc) | CD38 |
| Alemtuzumab | CD52 |
| Polatuzumab vedotin (polatuzumab vedotin-piiq) | CD79 |
| Ipilimumab | CTLA-4 |
| Tremelimumab | CTLA-4 |
| Cetuximab | EGFR |
| Necitumumab | EGFR |
| Nimotuzumab | EGFR |
| Panitumumab | EGFR |
| Amivantamab | EGFR, cMET |
| Edrecolomab | EpCAM |
| Oportuzumab monatox | EpCAM |
| Catumaxomab | EPCAM, CD3 |
| Mirvetuximab soravtansine | FOLR1 |
| Dinutuximab | GD2 |
| Naxitamab-gqgk | GD2 |
| Racotumomab | GM3 |
| [fam-]trastuzumab deruxtecan, | HER2 |
| (fam-trastuzumab deruxtecan-nxki) | |
| Ado-trastuzumab emtansine | HER2 |
| Disitamab vedotin | HER2 |
| Inetetamab | HER2 |
| Margetuximab-cmkb | HER2 |
| Pertuzumab | HER2 |
| Trastuzumab | HER2 |
| Teprotumumab (teprotumumab-trbw) | IGF-1R |
| Relatlimab | LAG-3 |
| Enfortumab vedotin (enfortumab vedotin-ejfv) | Nectin-4 |
| Olaratumab | PDGFRα |
| Adebrelimab | PD-L1 |
| Atezolizumab | PD-L1 |
| Avelumab | PD-L1 |
| Durvalumab | PD-L1 |
| Envafolimab | PD-L1 |
| Socazolimab | PD-L1 |
| Sugemalimab | PD-L1 |
| Tagitanlimab | PD-L1 |
| Elotuzumab | SLAMF7 |
| Sacituzumab govitecan (sacituzumab govitecan-hziy) | TROP-2 |
| Ramucirumab | VEGFR2 |

In some embodiments, the antigen is epidermal growth factor receptor (EGFR). In some embodiments, the second antibody is selected from: cetuximab, panitumumab and necitumumab. In some embodiments, the second antibody is cetuximab. In some embodiments, the second antibody is panitumumab. In some embodiments, the second antibody is necitumumab. In some embodiments, cetuximab comprises a heavy chain comprising SEQ ID NO: 1069 or analogs or homologs comprising at least 85% sequence identity. In some embodiments, cetuximab comprises a light chain comprising SEQ ID NO: 1068 or analogs or homologs comprising at least 85% sequence identity. In some embodiments, panitumumab comprises a heavy chain comprising SEQ ID NO: 1071 or analogs or homologs comprising at least 85% sequence identity. In some embodiments, panitumumab comprises a light chain comprising SEQ ID NO: 1070 or analogs or homologs comprising at least 85% sequence identity. In some embodiments, necitumumab comprises a heavy chain comprising SEQ ID NO: 1073 or analogs or homologs comprising at least 85% sequence identity. In some embodiments, necitumumab comprises a light chain comprising SEQ ID NO: 1072 or analogs or homologs comprising at least 85% sequence identity. In some embodiments, the analog or homolog retains target binding function. In some embodiments, the analog or homolog retains the CDRs of the antibody. In some embodiments, the CDRs are all of the CDRs that are not inert.

In some embodiments, at least one inert CDR of the second antibody is replaced with an immunogenic peptide. In some embodiments, the second antibody is a targeting antibody. In some embodiments, CDRH1 of cetuximab is replaced. In some embodiments, CDRL1 of cetuximab is replaced. In some embodiments, CDRL2 of cetuximab is replaced. In some embodiments, CDRL1 of panitumumab is replaced. In some embodiments, CDRL2 of panitumumab is replaced. In some embodiments, CDRL1 of necitumumab is replaced. In some embodiments, CDRL2 of necitumumab is replaced. In some embodiments, the CDR is replaced with a peptide selected from those provided in Tables 1-3. In some embodiments, the CDR is replaced with a peptide selected from those provided in Table 1. In some embodiments, the CDR is replaced with a peptide selected from those provided in Table 2. In some embodiments, the CDR is replaced with a peptide selected from those provided in Table 3. In some embodiments, the CDR is replaced with a peptide selected from SEQ ID NO: 1-11. In some embodiments, the CDR is replaced with a peptide selected from SEQ ID NO: 1-5. In some embodiments, the CDR is replaced with SEQ ID NO: 1. In some embodiments, the second antibody comprises an immunogenic peptide in place of amino acid sequence from an inert CDR. In some embodiments, the inert CDRs are provided in Table 14.

In some embodiments, the first antibody comprises at least one modification that promotes heterodimerization. In some embodiments, the first antibody comprises at least one modification that inhibits homodimerization. In some embodiments, the second antibody comprises at least one modification that promotes heterodimerization. In some embodiments, the second antibody comprises at least one modification that inhibits homodimerization. In some embodiments, the modification are knob-in-holes modifications. In some embodiments, the modification is a mutation. In some embodiments, the modification is in the constant region. In some embodiments, the constant region is the Fc region.

In some embodiments, the Fc region comprises an Ig CH2 domain. In some embodiments, the Fc region comprises an Ig heavy chain CH2 domain. In some embodiments, the Fc region comprises an Ig CH3 domain. In some embodiments, the Fc region comprises an Ig heavy chain CH3 domain. In some embodiments, the Fc region comprises or consists of both an Ig CH2 domain and Ig CH3 domain. In some embodiments, the Fc region comprises or consists of both an Ig heavy chain CH2 and an Ig heavy chain CH3 domain. In some embodiments, the first chain comprises a first portion of an Fc region and the second chain comprises a second portion of the Fc region. In some embodiments, the first portion comprises a CH2 domain, a CH3 domain or both. In some embodiments, the second portion comprises a CH2 domain, a CH3 domain or both. In some embodiments, interface of the first portion of an Fc region and the second portion of an Fc region produces a functional Fc region. In some embodiments, interface comprises contact. In some embodiments, interface comprises adjacent positioning. In some embodiments, interface comprises formation of the protein complex of the invention. In some embodiments, interface comprises dimerization of the first and second dimerization domains. In some embodiments, the CH2 domain is an Ig CH2 domain. In some embodiments the CH2 domain is a heavy chain CH2 domain. In some embodiments, the CH3 domain is an Ig CH3 domain. In some embodiments, the CH3 domain is a heavy chain CH3 domain.

In some embodiments, a CH2 domain comprises the amino acid sequence SVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAK (SEQ ID NO: 1097) or analogs or homologs comprising at least 85% sequence identity. In some embodiments, the CH2 domain consists of SEQ ID NO: 1097. In some embodiments, SEQ ID NO: 1097 is the IgG1 CH2 domain.

In some embodiments, a CH3 domain comprises the amino acid sequence GQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 1098) or analogs or homologs comprising at least 85% sequence identity. In some embodiments, a CH3 domain comprises the amino acid sequence GQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 1099) or analogs or homologs comprising at least 85% sequence identity. In some embodiments, the CH3 domain consists of SEQ ID NO: 1098. In some embodiments, the CH3 domain consists of SEQ ID NO: 1099. In some embodiments, SEQ ID NO: 1098 is the IgG1 CH3 domain. In some embodiments, SEQ ID NO: 1099 is the IgG1 CH3 domain. In some embodiments, the SEQ ID NO: 1098 sequence is the sequence found predominantly is humans of European and American descent. In some embodiments, SEQ ID NO: 1099 is the sequence found predominantly in humans of Asian descent.

In some embodiments, a CH3 domain comprises a mutation. In some embodiments, the first CH3 domain comprises a first mutation. In some embodiments, the second CH3 domain comprises a second mutation. In some embodiments, a CH2 domain comprises a mutation. In some embodiments, the first CH2 domain comprises a first mutation. In some embodiments, the second CH2 domain comprises a second mutation. In some embodiments, the CH2 and CH3 domains both comprise mutations. In some embodiments, the first CH2 domain and first CH3 domains each comprise a first mutation. In some embodiments, the second CH2 domain and the second CH3 domain each comprise a second mutation. In some embodiments, the mutations inhibit homodimerization of the first polypeptide chain. In some embodiments, the first mutation inhibits homodimerization of the first polypeptide chain. In some embodiments, the mutations inhibit homodimerization of the second polypeptide chain. In some embodiments, the second mutation inhibits homodimerization of the second polypeptide chain. In some, embodiments, the mutations permit heterodimerization. In some embodiments, the mutations permit heterodimerization of the first and second chains. In some embodiments, permitting is promoting. In some embodiments, permitting is enhancing.

Mutations that promote heavy chain heterodimerization and/or inhibit homodimerization are well known in the art. Any such mutations or alterations may be used for constructing the polypeptides of the invention. In some embodiments, a region from an IgG is replaced with a region from an IgA. In some embodiments, a region from a TCRa is inserted into the first CH3 domain and a region from TCRb is inserted in to the second CH3 domain. In some embodiments, the mutation is insertion of a region from a TCR. In some embodiments, the TCR is selected from TCRa and TCRb. In some embodiments, the mutation is insertion of a region from a different Ig. Examples of these mutations can be found in Table 5. In some embodiments, the mutation is selected from a mutation in Table 5. In some embodiments, the first mutation is selected from a group of mutation provided in a row and the second column of Table 5 and the second mutation is the group of mutations provided in that same row of Table 5 in the third column. The mutations in Table 5 are provided with the Kabat numbering for IgG1 unless otherwise stated; corresponding mutations can be made in other IGs and specifically in other IgGs. In some embodiments, the first mutation is T366Y, and the second mutation is Y407T. In some embodiments, the first mutation is S354C and T366W and the second mutation is Y349C, T366S, L368A, and Y407V. In some embodiments, the first mutation is S364H and F405A and the second mutation is Y349T and T392F. In some embodiments, the first mutation is T350V, L351Y, F405A, and Y407V and the second mutation is T350V, T366L, K392L, and T394W. In some embodiments, the first mutation is K392D, and K409D and the second mutation is E356K, and D399K. In some embodiments, the first mutation is D221E, P228E, and L368E and the second mutation is D221R, P228R, and K409R. In some embodiments, the first mutation is K360E, and K409W and the second mutation is Q347R, D399V, and F405T. In some embodiments, the first mutation is K360E, K409W, and Y349C and the second mutation is Q347R, D399V, F405T, and S354C. In some embodiments, the first mutation is F405L and the second mutation is K409R. In some embodiments, the first mutation is K360D, D399M, and Y407A and the second mutation is E345R, Q347R, T366V, and K409V. In some embodiments, the first mutation is Y349S, K370Y, T366M, and K409V and the second mutation is E356G, E357D, S364Q, and Y407A. In some embodiments, the first mutation is T366K, and the second mutation is selected from C351D, Y349E, Y349D, L368E, L368D, Y349E and R355E, Y349E and R355D, Y349D and R355E, and Y349D and R355D. In some embodiments, the first mutation is T366K and C351K and the second mutation is selected from C351D, Y349E, Y349D, L368E, L368D, Y349E and R355E, Y349E and R355D, Y349D and R355E, and Y349D and R355D. In some embodiments, the first mutation is L351D and L368E and the second mutation is L351K and T366K. In some embodiments, the first mutation is L368D and K370S and the second mutation is E357Q and S364K. In some embodiments, the first mutation is T366W, and the second mutation is T366S, L368A and Y407V. In some embodiments, the Ig is IgG2, and the first mutation is C223E, P228E, and L368E and the second mutation is C223R, E225R, P228R, and K409R. In some embodiments, the first mutation is S354C or T366W and the second mutation is Y349C, T366S, L368A, or Y407V. In some embodiments, the first mutation is S364H or F405A and the second mutation is Y349T or T392F. In some embodiments, the first mutation is T350V, L351Y, F405A, or Y407V and the second mutation is T350V, T366L, K392L, or T394W. In some embodiments, the first mutation is K392D, or K409D and the second mutation is E356K, or D399K. In some embodiments, the first mutation is D221E, P228E, or L368E and the second mutation is D221R, P228R, or K409R. In some embodiments, the first mutation is K360E, or K409W and the second mutation is Q347R, D399V, or F405T. In some embodiments, the first mutation is K360E, K409W, or Y349C and the second mutation is Q347R, D399V, F405T, or S354C. In some embodiments, the first mutation is K360D, D399M, or Y407A and the second mutation is E345R, Q347R, T366V, or K409V. In some embodiments, the first mutation is Y349S, K370Y, T366M, or K409V and the second mutation is E356G, E357D, S364Q, or Y407A. In some embodiments, the first mutation is L351D or L368E and the second mutation is L351K or T366K. In some embodiments, the first mutation is L368D or K370S and the second mutation is E357Q or S364K. In some embodiments, the first mutation is T366W, and the second mutation is T366S, L368A or Y407V. In some embodiments, the Ig is IgG2, and the first mutation is C223E, P228E, or L368E and the second mutation is C223R, E225R, P228R, or K409R. In some embodiments, the first heavy chain constant region comprises or consists of SEQ ID NO: 1074 or analogs or homologs comprising at least 85% sequence identity. In some embodiments, the second heavy chain constant region comprises or consists of SEQ ID NO: 1074. In some embodiments, the first heavy chain constant region comprises or consists of SEQ ID NO: 1075 or analogs or homologs comprising at least 85% sequence identity. In some embodiments, the second heavy chain constant region comprises or consists of SEQ ID NO: 1075. It will be understood that SEQ ID NO: 1074 and SEQ ID NO: 1075 heterodimerize with each other, but inhibit homodimerization. In some embodiments, the CL domain comprises or consists of SEQ ID NO: 1076 or analogs or homologs comprising at least 85% sequence identity. In some embodiments, the light chain constant region comprises or consists of SEQ ID NO: 1076. In some embodiments, the analog or homolog retains the mutations that promote heterodimerization and inhibit homodimerization.

TABLE 5
Mutations for enhancing heterodimerization and
inhibiting homodimerization of CH3 domains.

| | Strategy | CH3 domain Chain 1 | CH3 domain Chain 2 |
|---|---|---|---|
| 1 | Knobs-into-holes (Y-T) | T366Y | Y407T |
| 2 | Knobs-into-holes (CW- | S354C, T366W | Y349C, T366S, L368A, |
| | CSAV) | | Y407V |
| 3 | HA-TF | S364H, F405A | Y349T, T394F |
| 4 | ZW1 (VYAV-VLLW) | T350V, L351Y, F405A, | T350V, T366L, K392L, |
| | | Y407V | T394W |
| 5 | CH3 charge pairs (DD- | K392D, K409D | E356K, D399K |
| | KK) | | |
| 6 | Hinge/CH3 charge (EEE- | D221E, P228E, L368E | D221R, P228R, K409R |
| | RRR) | | |
| 7 | EW-RVT | K360E, K409W, | Q347R, D399V, F405T |
| 8 | EW-RVTS-S | K360E, K409W, Y349C | Q347R, D399V, F405T, |
| | | | S354C |
| 9 | (L-R) | F405L | K409R |
| 10 | 7.8.60 (DMA-RRVV) | K360D, D399M, | E345R, Q347R, T366V, |
| | | Y407A | K409V |
| 11 | 20.8.34 (SYMV-GDQA) | Y349S, K370Y, | E356G, E357D, S364Q, |
| | | T366M, K409V | Y407A |
| 12 | Electrostatic steering | 366K or 366K+ C351K | C351D or E or D at 349, |
| | effects | | 368, 349, or 349 +355 |
| 13 | “DEKK” | L351D and L368E | L351K and T366K |
| 14 | XmAb | L368D/K370S | E357Q/S364K |
| 15 | KiH | T366W | T366S/L368A/Y407V |
| 16 | IgG2 hinge/CH3 charge | IgG2: C223E, P228E, | IgG2: C223R, E225R, |
| | (EEE-RRRR) | L368E | P228R, K409R |
| 17 | SEEDbody | IgG/A chimera | IgG/A chimera |
| 18 | BEAT | residues from TCRa | residues from TCRb |
| | | interface | interface |

In some embodiments, the dual-function antigen binding molecule comprises two heavy chains and two light chains. In some embodiments, the two heavy chains are SEQ ID NO: 1088 and 1080 and the two light chains are SEQ ID NO: 1087 and 1079 or analogs or homologs comprising at least 85% sequence identity. In some embodiments, the two heavy chains are SEQ ID NO: 1088 and 1082 and the two light chains are SEQ ID NO: 1087 and 1081 or analogs or homologs comprising at least 85% sequence identity. In some embodiments, the two heavy chains are SEQ ID NO: 1090 and 1082 and the two light chains are SEQ ID NO: 1089 and 1079 or analogs or homologs comprising at least 85% sequence identity. In some embodiments, the two heavy chains are SEQ ID NO: 1090 and 1082 and the two light chains are SEQ ID NO: 1089 and 1081 or analogs or homologs comprising at least 85% sequence identity. In some embodiments, the two heavy chains are SEQ ID NO: 1088 and 1086 and the two light chains are SEQ ID NO: 1087 and 1085 or analogs or homologs comprising at least 85% sequence identity. It will be understood that analogs or homologs will target to the target protein by the targeting module, bind the cell and be delivered to the cytosol by the killing module and the will contain the immunogenic peptide which is displayed on the cell surface in complex with HLA.

In some embodiments, the composition is a pharmaceutical composition. In some embodiments, the composition comprises a pharmaceutically acceptable carrier, excipient or adjuvant. In some embodiments, the composition if formulated for administration to a subject. In some embodiments, the composition is formulated for systemic administration. In some embodiments, the composition if formulated for administration to a tumor. In some embodiments, the composition is formulated for intravenous administration. In some embodiments, the composition is formulated for administration to a subject. In some embodiments, the subject is a human.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, intratumoral or intraperitoneal.

In some embodiments, the nucleic acid molecule comprises an open reading frame. In some embodiments, the open reading frame encodes the antigen binding molecule of the invention. In some embodiments, the nucleic acid molecule comprises a plurality of open reading frames which collectively encode the antigen binding molecule of the invention.

In some embodiments, the vector is an expression vector. In some embodiments, the vector comprises at least one regulatory element operatively linked to a nucleic acid molecule of the invention. In some embodiments, the vector comprises at least one regulatory element operatively linked to an open reading frame encoding the antigen binding molecule of the invention. In some embodiments, the vector comprises a plurality of regulatory elements each operatively linked to an open reading frame which collectively encode the antigen binding molecule of the invention. In some embodiments, a composition comprises a plurality of vectors each comprising at least one regulatory element operatively linked to an open reading frame wherein the plurality of open reading frames collectively encodes the antigen binding molecule of the invention.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing of a gene within a cell is well known to one skilled in the art. It can be carried out by, among many methods, transfection, viral infection, or direct alteration of the cell's genome. In some embodiments, the gene is in an expression vector such as plasmid or viral vector.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the gene is operably linked to a promoter. The term “operably linked” is intended to mean that the nucleotide sequence of interest is linked to the regulatory element or elements in a manner that allows for expression of the nucleotide sequence (e.g. in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, the method is a method of treating cancer. In some embodiments, the antigen binding molecule of the invention is for use in treating cancer. In some embodiments, the dual function antigen binding molecule of the invention is for use in treating cancer. In some embodiments, the composition of the invention is for use in treating cancer. In some embodiments, the cancer is a cancer that expresses the cancer specific antigen. In some embodiments, the cancer is a cancer that overexpresses the cancer specific antigen. In some embodiments, overexpresses is expresses at a level higher than in non-cancerous cells. In some embodiments, the non-cancerous cells are of the same cell type or tissue as the cancerous cells. In some embodiments, the cancer is a cancer that expresses the immunogenic cancer peptide. In some embodiments, the cancer is a hematopoietic cancer. In some embodiments, the cancer comprises a malignant immune cell. In some embodiments, the immune cell is a B cell. In some embodiments, the cancer is a solid cancer. In some embodiments, the cancer is a PD-L1 positive cancer. In some embodiments, the cancer is an EGFR positive cancer. In some embodiments, the cancer is an EGFR overexpressing cancer.

As used herein “cancer” refers to diseases associated with cell proliferation. Non-limiting types of cancer include carcinoma, sarcoma, lymphoma, leukemia, blastoma and germ cells tumors. In one embodiment, carcinoma refers to tumors derived from epithelial cells including but not limited to breast cancer, prostate cancer, lung cancer, pancreas cancer, and colon cancer. In one embodiment, sarcoma refers of tumors derived from mesenchymal cells including but not limited to sarcoma botryoides, chondrosarcoma, ewings sarcoma, malignant hemangioendothelioma, malignant schwannoma, osteosarcoma and soft tissue sarcomas. In one embodiment, lymphoma refers to tumors derived from hematopoietic cells that leave the bone marrow and tend to mature in the lymph nodes including but not limited to hodgkin lymphoma, non-hodgkin lymphoma, multiple myeloma and immunoproliferative diseases. In one embodiment, leukemia refers to tumors derived from hematopoietic cells that leave the bone marrow and tend to mature in the blood including but not limited to acute lymphoblastic leukemia, chronic lymphocytic leukemia, acute myelogenous leukemia, chronic myelogenous leukemia, hairy cell leukemia, T-cell prolymphocytic leukemia, large granular lymphocytic leukemia and adult T-cell leukemia. In one embodiment, blastoma refers to tumors derived from immature precursor cells or embryonic tissue including but not limited to hepatoblastoma, medulloblastoma, nephroblastoma, neuroblastoma, pancreatoblastoma, pleuropulmonary blastoma, retinoblastoma and glioblastoma-multiforme. In one embodiment, germ cell tumors refers to tumors derived from germ cells including but not limited to germinomatous or seminomatous germ cell tumors (GGCT, SGCT) and nongerminomatous or nonseminomatous germ cell tumors (NGGCT, NSGCT). In one embodiment, germinomatous or seminomatous tumors include but not limited to germinoma, dysgerminoma and seminoma. In one embodiment, nongerminnomatous or nonseminomatous tumors refers to pure and mixed germ cells tumors including but not limited to embryonal carcinoma, endodermal sinus tumor, choriocarcinoma, tearoom, polyembryoma, gonadoblastoma and teratocarcinoma.

In some embodiments, the antigen binding molecule of the invention is a cancer vaccine. In some embodiments, cancer vaccine is an antigen binding molecule comprising an antigen binding region capable of binding a dendritic cell antigen. It will be understood by a skilled artisan that upon entrance into a dendritic cell, the immunogenic cancer peptide will be cleaved from the rest of the molecule of the invention and displayed on the surface of the dendritic cell by HLA molecules. This will in turn train cytotoxic immune cells (T cell and NK cells) to target this immunogenic peptide and thereby the cancer.

In some embodiments, treating further comprises administering effector cells specific to the immunogenic peptide to the subject. In some embodiments, an effector cell is an immune cell. In some embodiments, an effector cell is a cytotoxic cell. In some embodiments, an effector cell is a lymphocyte. In some embodiments, an effector cell is a CD8 T cell. In some embodiments, an effector cell is a natural killer (NK) cell. In some embodiments, the effector cell has been exposed to the peptide. In some embodiments, the effector cell has been exposed to an antigen presenting cell presenting the peptide in complex with an HLA.

In some embodiments, the treating further comprises providing a vaccine comprising the immunogenic peptide. In some embodiments, the subject has previously received a vaccine comprising the immunogenic peptide. In some embodiments, the subject is a mammal. In some embodiments, the subject is a human. In some embodiments, the subject suffers from cancer. In some embodiments, the subject is suitable to be treated by a method of the invention. In some embodiments, the subject has previously been exposed to the immunogenic peptide. In some embodiments, the subject has previously been infected by the pathogen from which the immunogenic peptide originates. In some embodiments, a pathogen is a virus. In some embodiments, the subject is capable of mounting an immune response against the peptide. In some embodiments, the subject comprises T cells comprising a TCR that recognizes the peptide or a portion thereof. In some embodiments, the subject comprises memory B cells comprising a BCR that recognizes the peptide or a portion thereof.

In some embodiments, the method is a method of producing surface display of the peptide in a target cell. In some embodiments, the surface display is display of HLA complexed with the peptide. In some embodiments, expressing is surface display. In some embodiments, expressing is expressing in an HLA complex.

By another aspect, there is provided a method of engineering an antibody or antigen binding fragment thereof, the method comprising:
a. selecting an antibody or antigen binding fragment thereof of interest;
b. determining at least one CDR of the selected antibody or antigen binding fragment thereof is not required for binding to a target;
c. replacing the determined at least one CDR or a portion thereof with a peptide;
   thereby engineering an antibody or antigen binding fragment thereof.

By another aspect, there is provided a method of engineering an antibody or antigen binding fragment thereof, the method comprising:
a. selecting an antibody or antigen binding fragment thereof of interest;
b. receiving a database of peptides;
c. performing alignment of peptides of a variable region of the selected antibody or antigen binding fragment thereof with peptides of the database;
d. determining a peptide from the selected antibody of antigen binding fragment thereof and a peptide from the database with an alignment score above a predetermined threshold; and
e. replacing the determined peptide from the selected antibody or antigen binding fragment thereof with the determined peptide from the database;
   thereby engineering an antibody or antigen binding fragment thereof.

In some embodiments, the engineered antibody or antigen binding fragment thereof is an antibody or antigen binding fragment of the invention. In some embodiments, the engineered antibody or antigen binding fragment thereof is an antigen binding molecule of the invention. In some embodiments, the engineered antibody or antigen binding fragment thereof is a dual-function antigen binding molecule of the invention. In some embodiments, the engineered antibody or antigen binding fragment thereof is an immunogenic peptide delivery antibody. In some embodiments, the engineered antibody or antigen binding fragment thereof is for use in a method of the invention. In some embodiments, the antibody or antigen binding fragment thereof before engineering is a cell penetrating antibody. In some embodiments, the selecting is selecting an antibody that penetrates into cells to which it binds. In some embodiments, the antibody or antigen binding fragment thereof before engineering is a commercially available antibody.

In some embodiments, the antibody or antigen binding fragment thereof before engineering binds to the surface of a target cell. In some embodiments, the target cell is a cancer cell. In some embodiments, step (a) comprises selecting an antibody or antigen binding fragment thereof that binds to a surface of a target cell. In some embodiments, binding the surface is binding a surface antigen. In some embodiments, antibody or antigen binding fragment thereof before engineering is a DNA binding antibody. In some embodiments, antibody or antigen binding fragment thereof before engineering is endocytosed into the endocytic pathway of a cell. In some embodiments, antibody or antigen binding fragment thereof before engineering is transported into the cytoplasm of a cell to which it binds. In some embodiments, antibody or antigen binding fragment thereof before engineering is capable of endosomal escape. In some embodiments, antibody or antigen binding fragment thereof before engineering is delivered to the cytosol of a cell to which it binds. In some embodiments, the method further comprises inserting into the antibody or antigen binding fragment thereof a cell penetrating moiety. In some embodiments, the cell penetrating moiety is inserted before the immunogenic peptide. In some embodiments, the cell penetrating moiety is inserted after the immunogenic peptide. In some embodiments, the cell penetrating moiety is inserted concomitantly to the insertion of the immunogenic peptide. In some embodiments, step (a) comprises selecting an antibody or antigen binding fragment thereof that upon binding to a surface of a target cell is internalized and delivered to a cytosol of the target cell.

In some embodiments, the antibody or antigen binding fragment thereof before engineering penetrates/internalizes into a bound cell at a level comparable to any one of TMab4, 3E10 and 71F12. In some embodiments, the antibody or antigen binding fragment thereof before engineering penetrates/internalizes into a bound cell at a level comparable to TMab4. In some embodiments, the antibody or antigen binding fragment thereof before engineering penetrates/internalizes into a bound cell at a level comparable to 3E10. In some embodiments, the antibody or antigen binding fragment thereof before engineering penetrates/internalizes into a bound cell at a level comparable to 71F12. In some embodiments, comparable is with a penetrance that is at least 50, 55, 60, 65, 70, 75, 80, 85, 90, 92, 95, 97, 99 or 100% of the original antibody. Each possibility represents a separate embodiment of the invention. In some embodiments, comparable is with a penetrance that is at least 80% of the original antibody. In some embodiments, comparable is with a penetrance that is at least 90% of the original antibody.

In some embodiments, the determining is determining at least one inert CDR. In some embodiments, the determining is determining at least one CDR not involved in transport into the cell. In some embodiments, the determining is determining at least one CDR not involved in endosomal escape. In some embodiments, the determining is based on structural analysis of the antibody or antigen binding fragment thereof bound to its target. In some embodiments, the structural analysis is in silico analysis. In some embodiments, the structural analysis is crystallographic analysis. In some embodiments, the method further comprises receiving the structural analysis. In some embodiments, the method further comprises performing the structural analysis. In some embodiments, the analysis provides the distance of each amino acid of the antibody or antigen binding fragment thereof to an amino acid of the target. In some embodiments, the analysis provides the distance of each amino acid of a CDR of the antibody or antigen binding fragment thereof to an amino acid of the target. In some embodiments, the determining comprises determining the distance of each amino acid of a CDR to the target. In some embodiments, a CDR with two or fewer amino acids in contact with the antigen is determined as not required for binding. In some embodiments, the determining comprises determining the distance of each amino acid of a CDR to the target. In some embodiments, a CDR with one or fewer amino acids in contact with the antigen is determined as not required for binding. In some embodiments, a CDR not required for binding is an inert CDR. In some embodiments, a CDR not required for binding is a CDR in which mutations have been shown not to effect antibody function. In some embodiments, the function is binding.

In some embodiments, the peptide is an immunogenic peptide. In some embodiments, the peptide comprises at least 5, 6, 7, 8, 9, 10, or 11 amino acids. Each possibility represents a separate embodiment of the invention. In some embodiments, the peptide comprises at least 7 amino acids. In some embodiments, the peptide comprises at least 8 amino acids. In some embodiments, the peptide comprises between 8 and 11 amino acids. In some embodiments, the peptide is an immunogenic peptide provided hereinabove. In some embodiments, peptide is from the variable region of the antibody or antigen binding fragment thereof. In some embodiments, peptide is from a CDR of the antibody or antigen binding fragment thereof. Methods of determining the position of CDRs are well known in the art and the Clothia and Kabat systems may be used. Given the sequence of a variable domain of an antibody a skilled artisan will be readily able to identify the CDRs.

In some embodiments, the at least one CDR is removed. In some embodiments, a portion of the at least one CDR is removed. In some embodiments, the removed amino acids are replaced with the immunogenic peptide. In some embodiments, the replacing is optimized. In some embodiments, the method further comprises optimizing the replacement. In some embodiments, optimizing is structural optimization. In some embodiments, optimizing comprises performing an optimization algorithm. In some embodiments, optimizing comprises producing as little perturbation in the structure of the selected antibody or antigen binding fragment thereof as possible. In some embodiments, optimizing comprises in silico analysis or the insertion of the immunogenic peptide into the at least one determined CDR with removal of all possible portions of the CDR and selected the insertion and removal of a portion that produces the least perturbation. In some embodiments, optimization comprise insertion of at least one filler amino Acid. some embodiments, optimization comprise producing at least one compensatory mutation outside the determined CDR. In some embodiments, the optimization algorithm is a minimal perturbation replacement (MBR) algorithm. In some embodiments, optimization comprises optimization of CDR flanking sequence. In some embodiments, optimization of flanking sequence comprises antibody stem preservation. In some embodiments, the structure of the stems flanking the CDRs are maintained. In some embodiments, the stems are the ends of the beta sheets flanking the CDR. In some embodiments, the end comprises the 1, 2, 3, 4, or 5 amino acids directly adjacent to the CDR. Each possibility represents a separate embodiment of the invention.

In some embodiments, the method further comprises confirming binding of the engineered antibody or antigen binding fragment thereof to the same target that was bound by the selected antibody or antigen binding fragment thereof. In some embodiments, the method further comprises measuring binding of the engineered antibody or antigen binding fragment thereof to the same target that was bound by the selected antibody or antigen binding fragment thereof. In some embodiments, the method further comprises determining binding of the engineered antibody or antigen binding fragment thereof to the same target that was bound by the selected antibody or antigen binding fragment thereof. In some embodiments, binding is equivalent to the binding of the selected antibody or antigen binding fragment thereof. In some embodiments, the measuring further comprises determining that the binding is not significantly reduced as compared to the binding of the selected antibody or antigen binding fragment thereof. In some embodiments, significantly is statistically significantly. In some embodiments, significantly reduced comprises a greater than 10% reduction. In some embodiments, significantly reduced comprises a greater than 20% reduction. In some embodiments, significantly reduced comprises a greater than 50% reduction. In some embodiments, significantly reduced is abolished.

In some embodiments, the method further comprises measuring levels of peptide in the cytosol of the target cell. In some embodiments, the method further comprises determining delivery of the peptide to the cytosol. In some embodiments, the method further comprises measuring delivery of the peptide to the cytosol.

In some embodiments, the method further comprises confirming delivery of the peptide to a cytosol of the target cell. In some embodiments, the method further comprises measuring levels of peptide in the cytosol of the target cell. In some embodiments, the method further comprises determining delivery of the peptide to the cytosol. In some embodiments, the method further comprises measuring delivery of the peptide to the cytosol.

In some embodiments, the method further comprises confirming delivery of the peptide to the surface of the target cell. In some embodiments, delivery is surface display of the peptide. In some embodiments, delivery is delivery of the peptide in complex with an HLA molecule to the surface of the target cell. In some embodiments, the method further comprises measuring levels of peptide on the surface of the target cell. In some embodiments, levels is levels of the peptide in complex with HLA. In some embodiments, the method further comprises determining delivery of the peptide to the surface of the target cell. In some embodiments, the method further comprises measuring delivery of the peptide to the surface of the target cell.

In some embodiments, the method further comprises confirming killing of the target cell by effector cells. In some embodiments, killing is specific killing. In some embodiments, effector cells are specific to the peptide. In some embodiments, effector cells are immune cells. In some embodiments, the method further comprises measuring killing of the target cell by effector cells. In some embodiments, the method further comprises determining killing of the target cell by effector cells. In some embodiments, the method is an in vitro method. In some embodiments, the confirming, measuring and determining is performed in vitro. In some embodiments, in vitro is ex vivo. Examples of methods of performing the confirming, measuring and determining are provided hereinbelow, but any assay known in the art for such measuring/confirming/determining may be used.

Databases of peptides and specifically immunogenic peptides are known in the art and any such database may be used. In some embodiments, the peptide is a cancer peptide. In some embodiments, database comprises or consists of Table 3. In some embodiments, the alignment is a pairwise alignment. In some embodiments, the alignment is alignment of one peptide from the selected antibody or antigen binding fragment thereof and one peptide of the database to produce an alignment pair. In some embodiments, an alignment pair with an alignment score above a predetermined threshold is used for replacement in step (e)

In some embodiments, the method further comprises inserting the engineered antibody into a dual-function antigen binding molecule. In some embodiments, the dual-function antigen binding molecule is a molecule of the invention. In some embodiments, the method further comprises selecting a targeting antibody that binds to a protein on a target cell. In some embodiments, on a target cell is on the surface of a target cell. In some embodiments, the target cell is a cancer cell. In some embodiments, the protein is a receptor. In some embodiments, the protein is a cancer antigen. In some embodiments, the protein is a protein only expressed on cancer cells and not counterpart healthy cell. In some embodiments, the protein is a protein overexpressed on cancer cells as compared to counterpart healthy cells. In some embodiments, counterpart cell are cells of the same tissue or cell type as the cancer cells. In some embodiments, counterpart cells are control cells. In some embodiments, the selected targeting antibody is combined with the engineered antibody. In some embodiments, the combining produces the dual-function antigen binding molecule. In some embodiments, a dual-function antigen binding molecule is a bi-specific antibody.

In some embodiments, the engineered antibody comprises one heavy chain and one light chain. In some embodiments, the targeting antibody or antigen binding fragment comprises one heavy chain and one light chain. In some embodiments, the engineered antibody is a single chain antibody. In some embodiments, the targeting antibody is a single chain antibody. In some embodiments, a single chain antibody comprises a heavy chain variable domain and a light chain variable domain linked in a single polypeptide by an amino acid linker. In some embodiments, the heavy chain constant region of the targeting antibody is engineered to promote heterodimerization and/or inhibit homodimerization. In some embodiments, the heavy chain constant region of the engineered antibody is engineered to promote heterodimerization and/or inhibit homodimerization. In some embodiments, engineered is modified. In some embodiments, modified is mutated. In some embodiments, the method comprises engineering the constant regions of the engineered antibody and/or the targeting antibody. In some embodiments, the engineering is producing a set of mutations provided in Table 5. In some embodiments, the heavy chain constant regions used comprise or consist of SEQ ID NO: 1074 and 1075 or analogs or homologs comprising at least 85% sequence identity. In some embodiments, the analogs or homologs retain the knob-in-holes mutations.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Cell penetration assay: The penetration ability of all Abs is screened by intracellular antibody detection followed by flow cytometry analysis. A fluorescent labeled anti-Fc-FITC is used to detect intracellular antibody. Briefly, breast cancer cells are seeded in 24 well plates and incubated for 6 hours with 0.5-1 uM of either control antibody or Trojan Ab. Next, cells are washed with PBS and low pH buffer (Glycine pH=2.5) to remove antibodies that are bound on the cell surface. The cells are then fixed, permeabilized and intracellularly fluorescently labeled by anti-Fc-FITC antibody (0.1% Saponin 1% BSA in PBS two hours at room temperature). Labeled cells are analyzed by flow cytometry for FITC and unlabeled cells are used as a negative control. To determine the level of proteasomal degradation of intracellular Trojan Abs a proteasomal inhibitor MG132 is added to cells prior to Trojan antibody addition.

Luciferase killing assay: Luciferase expressing target cancer cells (T) are seeded on 96-well plates and treated with the Trojan-Abs at various concentrations (0.5-4 μM). After 3-24 hours antigen-specific effector cells (E), specific to the relevant peptide within the Trojan-Ab, are added at various E:T ratios. Cells are then cocultured for 18-24 hours, followed by Luciferase substrate (Bio-Glo; Promega) addition. To quantify the number of viable cells, luminescence intensity (lum) is recorded. Killing is assessed by calculating % Killing=1−lum (Treated Target+Effectors)/Lum (Control non-Treated), this can also be calculated vs. other controls.

Image based killing assay (IncuCyte Imaging system): Target cells (T) are first labeled with a cytosolic red dye (IncuCyte® Cytolight Rapid Red) enabling tracking of target cell proliferation on the IncuCyte imager. Labeled target cell are then seeded on clear 96-well plates and treated with the Trojan-Abs at various concentrations (0.5-4 PM). After 3-24 hours antigen-specific effector cells (E), specific to the relevant peptide within the Trojan-Ab, are added at various E:T ratios. Next, plates are placed in the IncuCyte (37° C.; 5% CO2) and imaged at 2-hour intervals for 48 hours. Alternatively, a Caspase3/7 green dye was added to the media to enable apoptosis assessment of the target cells. Data is analyzed by the IncuCyte software analysis tool comparing two main parameters: Proliferation: The confluence of the red dye normalized to time 0 h; and Apoptosis: (Red+Green area)/Red-area normalized to time 0 h.

EGFR binding assay: To assess the ability of full Trojan antibodies (FTAbs) to bind EGFR on the cancer cell surface, a flow cytometry binding assay was established. Pre-plated cells are trypsinized, washed and then incubated on ice for 1 hour with 0.3 or 3 nM FTAb or EGFR bivalent therapeutic antibodies serving as a positive control. Then cells are washed with 2% FBS in PBS buffer three times and labeled with a fluorescent secondary antibody (anti-human Fc-FITC) for 30 minutes on ice. Cells are then washed with the same buffer three times and analyzed by flow cytometry. FTAbs binding is proportional to the FITC fluorescence level.

Presentation of HLA-peptide complex on the cancer cell surface: To quantify the level of presentation of HLA-peptide complex on the cancer cells upon incubation with FTAbs, TCR-like antibodies that are able bind specific HLA-peptide complex were used. Briefly, cells are seeded in 24 well plates and incubated for different time durations (4 h, 8 h and 10 h) with 1 M of FTAb or cFTAb (control FTAb) at 37C, 5% CO2. Next, cells are incubated on ice for 30 minutes with C1-17 antibody (see Lee et al. “Affinity Maturation of a T-Cell Receptor-Like Antibody Specific for a Cytomegalovirus pp65-Derived Peptide Presented by HLA-A*02:01” Int. J. Mol. Sci. 2021, 22, 2349, herein incorporated by reference in its entirety.) that is conjugated to fluorescein (FITC) and analyzed by flow cytometry.

### Example 1: Trojan Antibody Design

It is known that immunogenic peptides that are presented by cancer cells can lead to immune cell activation and cancer cell killing. Lymphocytes within the subject recognize the immunogenic peptide and the subject's immune system is brought to bear against the cancer cell. This mechanism can be harnessed by actively delivering the immunogenic peptide into cancer cells, who intern display the immunogenic peptide on their cell surface. Two classes of immunogenic peptides can be employed: 1) non-self-peptides/peptides not found in humans such as viral or bacterial peptides and 2) modified self-peptides which are human peptides that are mutated or otherwise altered to provide cancer specificity so that the immune system is not activated against healthy cells.

It has previously been hypothesized that the immunogenic peptide can be delivered via an antibody. Sefrin et al., 2019 “Sensitization of tumors for attack by virus-specific CD8+ T-cells through antibody-mediated delivery of immunogenic T cell epitopes”, Front. Immunol., August 21; 10:1962 made use of a full-sized antibody conjugated to a peptide via a disulfide bond. However, this construct may have very low stability, resulting in high rates of de-conjugation in serum. This in turn may lead to poor delivery to the target cells, reduced potency and high off target effects (side effects). Gaston et al., 2019, “Intracellular delivery of therapeutic antibodies into specific cells using antibody-peptide fusions”, Sci. Rep., December 10; 9(1):18688 employed a fusion protein construct in which the peptide was integrated into an end of the amino acid chain of the heavy or light chain or was placed next to the hinge domain within the chain. This method led to decreased production yield, increased aggregation and reduced in vitro stability.

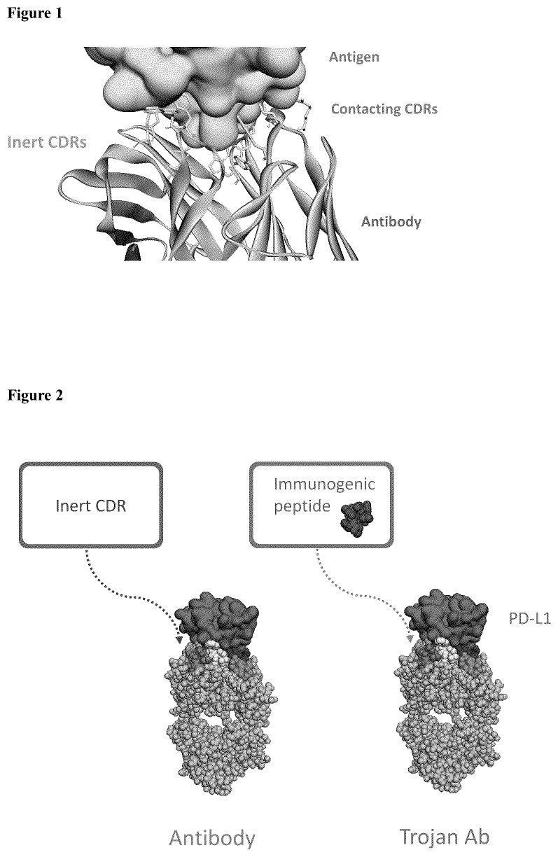

To alleviate the problems inherent to these methods, rather than insert the immunogenic peptide at the end of the chain or between structures the immunogenic peptide is inserted into the antibody variable region. In particular, the immunogenic peptide is use to replace all or part of “inert”, non-essential CDRs within the heavy or light chain of the antibody (FIG. 1). In silico modeling is used to identify inert CDR loops in known therapeutic antibodies. The inert CDR is then replaced by the immunogenic peptide (FIG. 2). In this conformation the engineered antibody maintains high stability and antigen binding affinity. If the immunogenic peptide is shorter than the CDR filler amino acids can be added so as not to change the antibody conformation. Similarly, if the CDR is longer than the immunogenic peptide only a portion of the CDR is removed. Flanking regions next to the inert CDRs can also be replaced but their overall structure should be conserved as these regions are often important for overall antibody conformation. These engineered antibodies are referred to as Trojan antibodies.

### Example 2: Trojan Antibody Uses

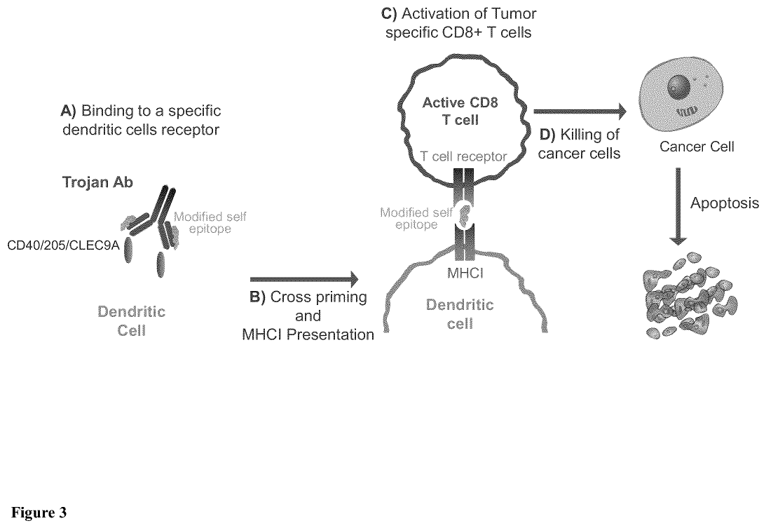

One use of the Trojan antibodies of the invention is dendritic cell vaccination. An antibody against a dendritic cell surface marker (e.g., CD40) is used for engineering. In this case anti-human CD40 antibody Fab516 is employed, and a modified self-antigen expressed by the target cancer is inserted into an inert CDR. After antibody binding to CD40, the antibody is digested and short peptides, including the immunogenic peptide, are displayed on the cell surface by MHCI molecules. CD8+ T cells are activated by these dendritic cells and become tumor specific leading to increased tumor cell killing (FIG. 3).

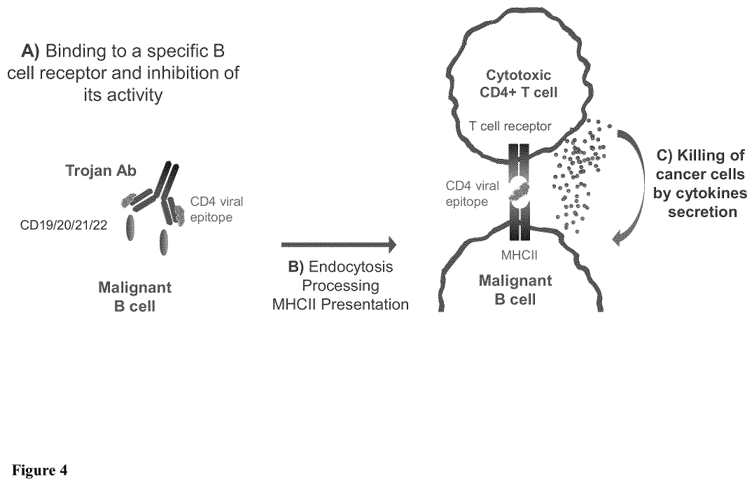

The Trojan antibodies of the invention are also used for treating B cell malignancies, such as leukemia. In this instance an antibody against a B cell surface marker (e.g., CD20) is used. For example, the Arzerra antibody has an inert CDR replaced with a viral epitope (e.g., a CD4 viral epitope/epitope recognized by CD4 T cells). After endocytosis, the antibody is processed and presented on the B cell's surface by MHCII molecules. Cytotoxic T cells recognize the viral epitope and kill the malignant cells (FIG. 4).

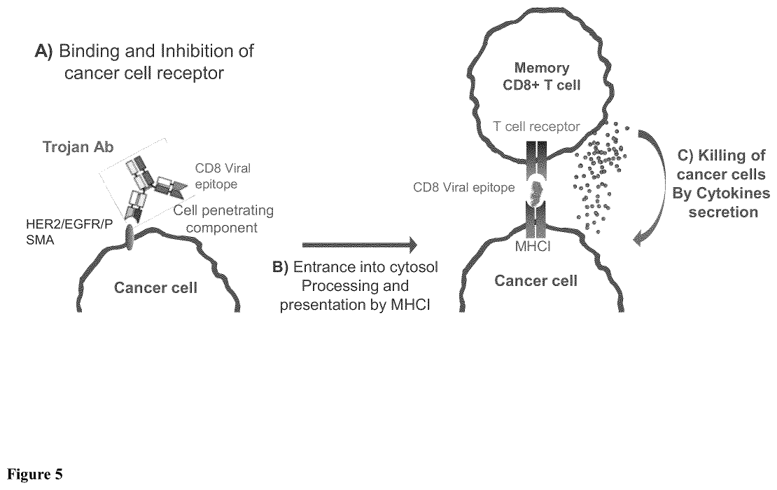

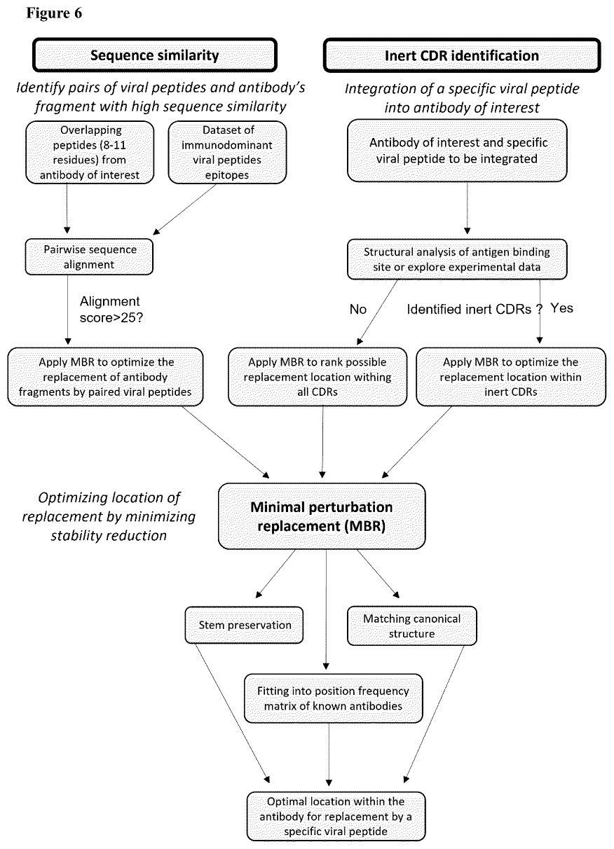

The Trojan antibodies of the invention are also used for treating solid tumors or general cancers. Antibodies that bind cancer specific epitopes such as HER2, EGFR, EpCAM and PSMA are used and in particular bi-specific antibodies are employed. In the case of a bi-specific molecule only one of the antigen binding domains is required to bring the agent to the cancer cell; the other can be substituted with one or more peptides recognized by CD8+ T cells and/or with a cell penetrating component. It will of course be understood that a cell penetrating component can also be inserted into a non-bispecific antibody if there are two inert CDRs (one for the penetrating component and one for the immunogenic peptide). Alternatively, an antibody can be used which is known to be internalized, so long as insertion of the immunogenic peptide does not interfere with internalization. After binding the target protein, the antibody is digested and released to the cytosol (endosomal escape). From there it is displayed on the cancer cell's surface via MHCII molecules (FIG. 5).

### Example 3: Trojan Antibody Pipeline with Inert CDR Identification (FIG. 6, Right Pathway)

Trojan antibodies are designed by first selecting an antibody of interest. Structural analysis of the binding of the antibody to its target is undertaken and inert CDRs are identified. An inert CDR is one that does not directly contact the protein target (a distance of at great than 5 angstroms from the protein target). A suitable immunogenic peptide that can replace the CDR is identified and inserted into the engineered antibody. The immunogenic peptide can be preselected, or an immunogenic peptide can be selected based on its similarity to the CRD being removed. Computational modeling is used to select the suitable peptide and confirm correct antibody conformation. This computational modeling is referred to as the minimal perturbation replacement (MBR) and it can be applied to optimize the replacement location within the CDR. For example, if the CDR is longer than the immunogenic peptide, filling amino acids may need to be added for minimal perturbation of antigen binding. Similarly, if the CDR is shorter than the immunogenic peptide, amino acids from the inter-CDR regions may also be removed. Flanking amino acids are often also involved in binding and antibody confirmation and structural similarity/minimal perturbation in the flanking regions is also desirous. The MBR optimizes the positioning of the immunogenic peptide.

If an antibody is selected without an obvious inert CDR, the MBR is applied to rank possible replacement locations within the antibody and specifically the CDRs. If a location can be found that does not perturb binding (or perturbs it by less than a predetermined threshold) than the immunogenic peptide is inserted in this location.

The MBR analysis takes into account the canonical structure of the antibody and selects positions/replacement location that most closely maintains this structure. Antibody stem preservation is highly conserved. The stem refers to the section of the beta-sheet directly adjacent to the CDR loop. This area is also referred to as the CDR flanking region. These 1-5 amino acids are important for loop position and tend to be highly rigid, whereas the loop itself, especially an inert CDR loop, may be less ordered. Stem preservation is thus important during MBR analysis. The MBR also takes into account the position frequency matrix of other known antibodies. The computational model has imputed hundreds of known antibodies and the frequency of each amino acid at each position is considered. Peptide replacement that most closely conforms to the positional frequency of the known antibodies is preferred. Finally, the MBR outputs the optimal location within the antibody for peptide replacement, or if there is more than one acceptable location it outputs a hierarchy positions.

### Example 4: Trojan Antibody Pipeline by Sequence Similarity (FIG. 6, Left Pathway)

Trojan antibodies can alternatively be generated based on sequence similarity. A target antibody is selected, as is a list of immunogenic peptides (see for example Tables 1-3). Pairwise sequence alignment is done between the various viral peptides and the antibody. Overlapping peptides within the antibody of 8-11 amino acids are compared with the immunogenic peptides and an alignment score is given for each pair (1 peptide from the antibody and one immunogenic peptide). The score was calculated based on blosum62 and a palanty of −3 per gap opening and per gap extension. A threshold was set for acceptable alignment (for example >25 was used in the below example) Immunogenic peptides and the peptides in the antibody to be replaced that meet the threshold for alignment score are selected and the MBR is applied to optimize replacement position.

### Example 5: Trojan Antibody Functional Confirmation (Inert CDRs)

Replacement based on sequence similarity is not limited to CDRs but can be done anywhere in the antibody. Because of the similarity in sequence, minimal perturbation is expected. Nevertheless, this method and the inert CDR replacement method still requires confirmation that the antibody has retained its functionality. In particular, it must be determined that the antibody will still bind to its target and is still internalized into cells. If binding/internalization is abolished than the new antibody is of no use.

The first antibody selected for insertion of an immunogenic peptide was a DNA hydrolyzing antibody 3D8, also known as TMab4. The mouse antibody 3D8 was first disclosed in Kim et al., “Heavy and Light Chain Variable Single Domains of an Anti-DNA Binding Antibody Hydrolyze Both Double- and Single-stranded DNAs without Sequence Specificity”, J Biol Chem., 2006, June 2; 281(22):15287-9 and its humanized form was provided in International Patent Application Wo2019/244086. TMab4 was shown to bind to and penetrate into cancer cells and reach the cytoplasm via endosomal escape, thus it was selected for conversion into a Trojan antibody. It was found that CDRs 1, 2 and 3 of the heavy chain (CDRH1, CDRH2, CDRH3) and CDR 3 of the light chain (CDRL3) were inert CDRs.

Initially, the ability to replace CDRH1 of TMab4 was tested. Two peptides from CMV, two peptides from EBV and one peptide from influenza were inserted into various positions in CDRH1 (see Table 6). Penetration superior to that of the negative control (Adalimumab) was retained for all inserted peptides regardless of positioning within CDRH1. However, some insertions showed superior penetrance, while others diminished penetrance as compared to the parental TMab4. Several of the constructs included the replacement of a flanking amino acids next to the CDR. This was done as these additional replacements were predicted to reduce perturbation of the overall antibody conformation. Specifically, in construct T1, the “S” directly N-terminal to the CDR was also replaced. In constructs T 32, T_33 and T_34 the “CAAS” directly N-terminal to the CDR was also replaced. In constructs T 42 and T_43 the “AAS” directly N-terminal to the CDR was also replaced. One construct, T1_34, included a compensatory mutation (T30S) in the heavy chain that was predicted to reduce perturbation of the antibody. Thus, though overall alteration of the inert CDR did not abolish penetrance, as expected, it did have an impact on the overall ability to enter the cell.

TABLE 6
Trojan antibodies with insertion into CDRH1 of TMab4. (PC)-positive
control; (NC)-Negative control; (Tab)-Trojan antibody; (The relative penetration of N1 is
given as an average of all replicates.)

| | PC/ | | | Location of | Sequence of | |
|---|---|---|---|---|---|---|
| | NC/ | Peptide | | replaced | replaced | Relative |
| Ab name | TAb | (SEQ ID NO:) | Location | fragment | fragment | penetration |
| P1 (TMab4) | PC | — | — | — | — | 100.00% |
| N1 | NC | — | — | — | — | 50.72% |
| (Adalimumab) | | | | | | |
| T1 | TAb | CMV | CDRH1 | VH 25-33 | SGYTFTSYV | 104.54% |
| | | NLVPMVATV | | | | |
| | | (1) | | | | |
| T2 | TAb | CMV | CDRH1 | VH 26-33 | GYTFTSYV | 84.06% |
| | | NLVPMVATV | | | | |
| | | (1) | | | | |
| T3 | TAb | CMV | CDRH1 | VH 26-32 | GYTFTSY | 96.92% |
| | | NLVPMVATV | | | | |
| | | (1) | | | | |
| T4 | TAb | CMV | CDRH1 | VH 27-33 | YTFTSYV | 81.45% |
| | | NLVPMVATV | | | | |
| | | (1) | | | | |
| T5 | TAb | CMV | CDRH1 | VH 28-33 | TFTSYV | 83.25% |
| | | NLVPMVATV | | | | |
| | | (1) | | | | |
| T1_32 | TAb | EBV | CDRH1 | VH 22-30 | CAASGYTFT | 89.85% |
| | | CLGGLLTMV | | | | |
| | | (2) | | | | |
| T1_33 | TAb | EBV | CDRH1 | VH 22-29 | CAASGYTF | 155.93% |
| | | CLGGLLTMV | | | | |
| | | (2) | | | | |
| T1_34 | TAb | EBV | CDRH1 | VH 22-29 | CAASGYTF | 106.10% |
| | | CLGGLLTMV | | | | |
| | | (2) | | | | |
| T1_35 | TAb | FLU | CDRH1 | VH 26-31 | GYTFTS | NA |
| | | GILGFVFTL (3) | | | | |
| T1_42 | TAb | EBV | CDRH1 | VH 23-32 | AASGYTFTSY | 115.37% |
| | | AVFDRKSDAK | | | | |
| | | (4) | | | | |
| T1_43 | TAb | EBV | CDRH1 | VH 23-31 | AASGYTFTS | 101.66% |
| | | AVFDRKSDAK | | | | |
| | | (4) | | | | |
| T1_46 | TAb | CMV | CDRH1 | VH 26-32 | GYTFTSY | 81.46% |
| | | QYDFVAALF | | | | |
| | | (5) | | | | |

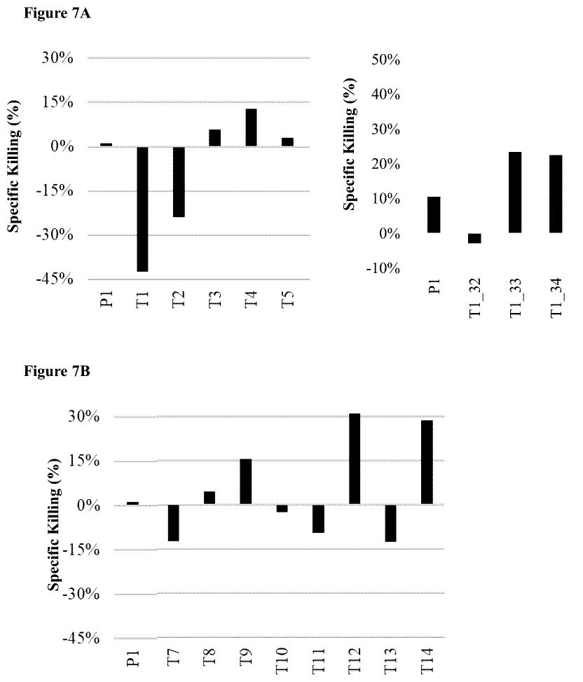

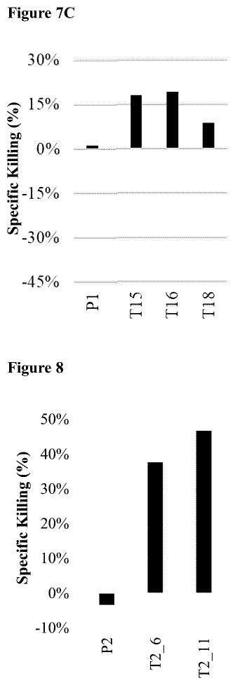

Several of the generated antibodies were tested for their ability to kill target cells in the presence of effector cells specific to the immunogenic peptide present in the Trojan antibody. As can be seen in FIG. 7A, though all antibodies were still able to penetrate the cell, the ability to induce specific killing was highly variable. Of the five insertions of the NLVPMVATV (SEQ ID NO: i) peptide two produced no specific killing and while the other three did With respect to peptide CLGGLLTMV (SEQ ID NO: 2) the two insertions that produced superior penetration also produced superior killing.

Next, insertion of SEQ ID NO: 1 and SEQ ID NO: 3 into CDRH3 was tested (Table 7). SEQ ID NO: 3 showed some structural similarity to the CDR it is replacing, in particular the “G” near the beginning of the CDR was maintained, and so it was tested along with SEQ ID NO: 1. It had previously been reported that CDRH3 was completely dispensable for antibody binding and cell penetration (see Lee et al., “Functional Consequences of Complementarity-determining Region Deactivation in a Multifunctional Anti-nucleic Acid Antibody”, J Biol Chem. 2013 Dec. 13; 288(50):35877-85, hereby incorporated by reference in its entirety.). However, insertion of SEQ ID NO: 1 greatly impaired penetrance such that it was only slightly superior to the negative control antibody. In contrast, insertion of SEQ ID NO: 3 invariably produced penetration with two of the insertion positions as good if not better than the parental antibody. This all suggests that selection of the immunogenic peptide to match the insertion location is highly important.

TABLE 7
Trojan antibodies with insertion into CDRH3 of TMab4. (PC)-positive
control; (NC)-Negative control; (Tab)-Trojan antibody; (The relative penetration of N1 is
given as an average of all replicates.)

| | PC/ | | | Location | Sequence of | |
|---|---|---|---|---|---|---|
| | NC/ | Peptide | | of replaced | replaced | Relative |
| Ab name | TAb | (SEQ ID NO:) | Location | fragment | fragment | penetration |
| P1 (TMab4) | PC | — | — | — | — | 100.00% |
| N1 | NC | — | — | — | — | 50.72% |
| (Adalimumab) | | | | | | |
| T7 | TAb | CMV | CDRH3 | VH 100-108 | AYKRGYAMD | 84.45% |
| | | NLVPMVATV (1) | | | | |
| T8 | TAb | CMV | CDRH3 | VH 99-106 | GAYKRGYA | 60.39% |
| | | NLVPMVATV (1) | | | | |
| T9 | TAb | CMV | CDRH3 | VH 99-105 | GAYKRGY | 53.97% |
| | | NLVPMVATV (1) | | | | |
| T10 | TAb | CMV | CDRH3 | VH 100-105 | AYKRGY | 59.90% |
| | | NLVPMVATV (1) | | | | |
| T11 | TAb | CMV | CDRH3 | VH 100-106 | AYKRGYA | 62.05% |
| | | NLVPMVATV (1) | | | | |
| T12 | TAb | CMV | CDRH3 | VH 101-105 | YKRGY | 51.82% |
| | | NLVPMVATV (1) | | | | |
| T13 | TAb | CMV | CDRH3 | VH 101-106 | YKRGYA | 57.99% |
| | | NLVPMVATV (1) | | | | |
| T14 | TAb | CMV | CDRH3 | VH 101-104 | YKRG | 63.08% |
| | | NLVPMVATV (1) | | | | |
| T1_39 | TAb | FLU | CDRH3 | VH 99-107 | GAYKRGYAM | 75.15% |
| | | GILGFVFTL (3) | | | | |
| T1_40 | TAb | FLU | CDRH3 | VH 99-106 | GAYKRGYA | 148.88% |
| | | GILGFVFTL (3) | | | | |
| T1_41 | TAb | FLU | CDRH3 | VH 99-105 | GAYKRGY | 94.74% |
| | | GILGFVFTL (3) | | | | |

Interestingly, when specific killing was tested with the Trojan antibodies containing SEQ ID NO: 1 inserted into CDRH3 are surprising result was observed. The two Tabs with the lowest measured penetrance demonstrated specific killing (FIG. 7B). It may be that the low detection within the cell was due to a rapid antibody processing and display on the cell surface leading to effective killing.

Insertion of SEQ ID NO: 1 and SEQ ID NO: 5 into CDRL3 and insertion of SEQ ID NO: 1 into CDRH2 was also tested (Table 8). SEQ ID NO: 5 had very high sequence alignment with CDRL3 and so was selected for testing. Several of the constructs included the replacement of a flanking amino acids next to the CDR. This was done as these additional replacements were predicted to reduce perturbation of the overall antibody conformation. Specifically, in constructs T1_44 and T1_45, the “F” directly C-terminal to the CDR was also replaced. In constructs T18 the “Y” directly C-terminal to the CDR was also replaced. All of the tested insertions produced good penetrance essentially equivalent to the parental antibody. This result is highly important as CDRL3 has been reported to be important for cell penetration, and this function was not perturbed by replacement of this CDR with the tested immunogenic peptides.

TABLE 8
Trojan antibodies with insertion into CDRL3 and CDRH2 of TMab4. (PC)-
positive control; (NC)-Negative control; (Tab)-Trojan antibody; (The relative penetration
of N1 is given as an average of all replicates.)

| | PC/ | | | Location of | Sequence of | |
|---|---|---|---|---|---|---|
| | NC/ | Peptide | | replaced | replaced | Relative |
| Ab name | TAb | (SEQ ID NO:) | Location | fragment | fragment | penetration |
| P1 (TMab4) | PC | — | — | — | — | 100.00% |
| N1 | NC | — | — | — | — | 50.72% |
| (Adalimumab) | | | | | | |
| T15 | TAb | CMV | CDRL3 | VL 97-103 | YYYHMYT | 103.87% |
| | | NLVPMVATV (1) | | | | |
| T16 | TAb | CMV | CDRL3 | VL 98-103 | YYHMYT | 92.94% |
| | | NLVPMVATV (1) | | | | |
| T1_44 | TAb | CMV | CDRL3 | VL 96-104 | QYYYHMYTF | 100.63% |
| | | QYDFVAALF (5) | | | | |
| T1_45 | TAb | CMV | CDRL3 | VL 98-104 | YYHMYTF | 94.69% |
| | | QYDFVAALF (5) | | | | |
| T18 | TAb | CMVNLVPMVATV | CDRH2 | VH 52-59 | NPYNDGNY | 111.90% |
| | | (1) | | | | |

Specific killing was tested for the Trojan antibodies containing SEQ ID NO: 1 and in this case all of the antibodies were found to induce killing (FIG. 7C). This indicates that this insertion produces highly measured penetrance and high levels of specific killing. Trojan antibodies are also generating containing a combination of inert CDR replacements. As 4 CDRs are inert, combinations of 2, 3 and 4 immunogenic peptides are generated. The inserted peptides can be repeats of the same peptide or different peptides.

A second DNA binding antibody capable of penetrating into cells was also tested. Antibody 3E10 (see Weisbart et al., “DNA-dependent targeting of cell nuclei by a lupus autoantibody”, Sci Rep. 2015 Jul. 9; 5:12022, hereby incorporated by reference in its entirety) was examined for inert CDRs and it was determined that CDRL1 and CDRL2 both did not engage the antigen during binding. SEQ ID NO: 1 was inserted into either CDRL1 or CDRL2 and SEQ ID NO: 3 was also inserted into CDRL1 (Table 9). One of the constructs included the replacement of a flanking amino acids next to the CDR. This was done as the additional replacements were predicted to reduce perturbation of the overall antibody conformation. Specifically, in construct T2_13, the “LLIK” directly N-terminal to the CDR and “YL” directly C-terminal to the CDR were also replaced. All of the SEQ ID NO: 1 insertions produced penetration above the negative control, although once again the levels of penetration varied.

TABLE 9
Trojan antibodies with insertion into CDRL1 and CDRL2 of 3E10. (PC)-
positive control; (NC)-Negative control; (Tab)-Trojan antibody.

| | PC/ | | | Location of | Sequence of | |
|---|---|---|---|---|---|---|
| | NC/ | Peptide | | replaced | replaced | Relative |
| Ab name | TAb | (SEQ ID NO:) | Location | fragment | fragment | penetration |
| P2 (3E10) | PC | — | — | — | — | 100.00% |
| N2 | NC | — | — | — | — | 54.70% |
| (Muromonab) | | | | | | |
| T2_6 | TAb | CMV | CDRL1 | VL 27-35 | KSVSTSSYS | 80.61% |
| | | NLVPMVATV (1) | | | | |
| T2_11 | TAb | CMV | CDRL1 | VL 28-36 | SVSTSSYSY | 98.50% |
| | | NLVPMVATV (1) | | | | |
| T2_12 | TAb | FLU | CDRL1 | VL 28-36 | SVSTSSYSY | X |
| | | GILGFVFTL (3) | | | | |
| T2_13 | TAb | CMV | CDRL2 | VL 50-58 | LLIKYASYL | 72.17% |
| | | NLVPMVATV (1) | | | | |

Killing with peptide specific effector cells was also tested for cancer cells treated with the Trojan antibodies derived from 3E10 and including SEQ ID NO: 1. Both tested TAbs produced robust specific killing (FIG. 8). These results demonstrate the universality of the Trojan system. Immunogenic peptides can be inserted into a variety of antibodies that can penetrate into cells and produce peptide specific killing.

All TAbs generated are cultured with cancer cells and penetrance is determined. Penetration is observed for all peptides which are determined to retain antibody conformation upon replacement of inert CDRs. All TAbs are tested for their ability to induce specific cancer cell killing in the presence of peptide specific effector cells. It will be understood by a skilled artisan that the specific type of cancer tested is not important as all cancer cells are able to bring HLA complex up to the cell surface and thereby display the immunogenic peptides of the TAbs. TAbs are able to produce enhanced specific cell killing above that of the parental antibody that does not have an immunogenic peptide. Trojan antibodies are also generating containing a combination of inert CDR replacements. As 2 CDRs are inert, combinations of two immunogenic peptides are generated. The inserted peptides can be repeats of the same peptide or different peptides.

### Example 6: Trojan Antibody Functional Confirmation (Sequence Similarity)

Replacement based on sequence similarity was also performed for the TMab4 antibody. A screen of immunogenic peptides (see Table 3) found only 0.0057% of tested peptides were above the alignment score threshold (>25) for possible replacement. The three with the most significant similarity to the TMab4 antibody were selected for replacement. SEQ ID NO: 6 from EBV corresponded to amino acids 28-35 within the heavy chain of the antibody. This includes two amino acids (“MH”) flanking the CDRH1 on its C-terminus. A compensatory mutation in the heavy chain (Y27D) was also made to reduce perturbation of the antibody. SEQ ID NO: 7 from SARS-CoV2 corresponded to amino acids 52-60 within the heavy chain of the antibody. This includes two amino acids (“YY”) flanking the CDRH2 on its C-terminus. SEQ ID NO: 8 from EBV corresponds to amino acids 14-22 in the first framework region on the light chain of the antibody. These three TAbs were generated, and penetrance was tested as before (Table 10). Although all three showed penetrance that was above that of the negative control SEQ ID NO: 7 and SEQ ID NO: 8 produced penetrance that was as good or better than the parental antibody, while SEQ ID NO: 6 produced worse penetrance.

TABLE 10
Trojan antibodies with insertion based on sequence similarity into TMab4.
(PC)-positive control; (NC)-Negative control; (Tab)-Trojan antibody; (The relative
penetration of N1 is given as an average of all replicates.)

| | PC/ | | | Location of | Sequence of | |
|---|---|---|---|---|---|---|
| | NC/ | Peptide | | replaced | replaced | Relative |
| Ab name | TAb | (SEQ ID NO:) | Location | fragment | fragment | penetration |
| P1 (TMab4) | PC | — | — | — | — | 100.00% |
| N1 | NC | — | — | — | — | 50.72% |
| (Adalimumab) | | | | | | |
| T19 | TAb | EBV | CDRH1 | VH 28-35 | TFTSYVMH | 53.97% |
| | | SVSSSISSL (6) | | | | |
| T1_30 | TAb | Cov2 | CDRH2 | VH 52-60 | NPYNDGNYY | 117.01% |
| | | LPFNDGVYF (7) | | | | |
| T1_31 | TAb | EBV | FW1L | VL 14-22 | SVGDRVTIT | 115.18% |
| | | FLGERVTLT (8) | | | | |

The sequence similarity method was also used to modify the 3E10 antibody. A screen of immunogenic peptides (see Table 3) found only 0.0093% of tested peptides were above the alignment score threshold (>25) for possible replacement. SEQ ID NO: 6 from EBV corresponded to amino acids 28-36 within CDRLS of the antibody. SEQ ID NO: 9 from SARS-CoV2 corresponded to amino acids 50-58 within the light chain of the antibody. This includes four amino acids (“LLIK”) flanking the CDRL2 on its N-terminus and two amino acids (“YL”) flanking the CDRL2 on its C-terminus. Both TAbs were generated and were found to have penetrance into cells that was comparable to the parental antibody (Table 11).

TABLE 11
Trojan antibodies with insertion based on sequence similarity into 3E10.
(PC)-positive control; (NC)-Negative control; (Tab)-Trojan antibody

| | PC/ | | | Location of | Sequence of | |
|---|---|---|---|---|---|---|
| | NC/ | Peptide | | replaced | replaced | Relative |
| Ab name | TAb | (SEQ ID NO:) | Location | fragment | fragment | penetration |
| P2 (3E10) | PC | — | — | — | — | 100.00% |
| N2 | NC | — | — | — | — | 54.70% |
| (Muromonab) | | | | | | |
| T2_20 | TAb | EBV | CDRL1 | VL 28-36 | SVSTSSYSY | 99.64% |
| | | SVSSSISSL (6) | | | | |
| T2_23 | TAb | COV2 | CDRL2 | VL 50-58 | LLIKYASYL | 98.32% |
| | | LLALHRSYL (9) | | | | |

Finally, a third antibody DNA binding antibody, 71F12 (see Sakakibara et al., “Clonal evolution and antigen recognition of anti-nuclear antibodies in acute systemic lupus erythematosus”, Sci Rep. 2017; 7: 16428, hereby incorporated by reference in its entirety), was analyzed for inert CDRs and it was determined that CDRL1 was inert. Substitutions were made based on sequence similarity and SEQ ID NO: 10 from Adenovirus corresponded to amino acids 26-34 within CDRL1 of the antibody (called T4_1). SEQ ID NO: 11 from HPV corresponds to amino acids 17-24 in the first framework region on the heavy chain of the antibody (called T4_3). Both TAbs are generated and are found to have penetrance into cells that was at least comparable to the parental antibody.

All generated TAbs are also tested for specific cell killing as described hereinabove. TAbs that successfully enter the cell are able to induce cell killing that is superior to the parental antibody that does not include killing.

### Example 7: Further Confirmation of Trojan Antibody Killing

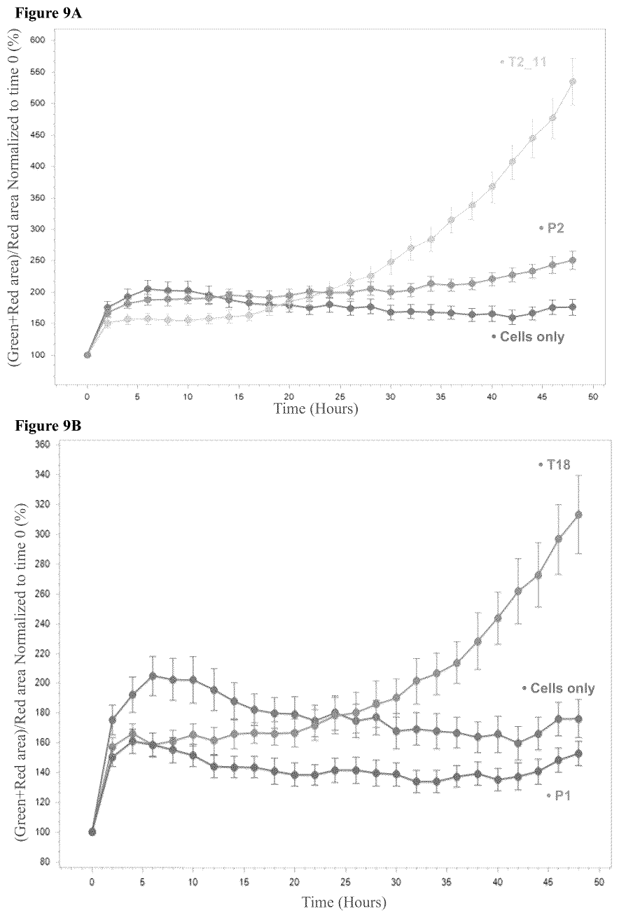

Killing by the Trojan antibodies was confirmed by a second method. An IncuCyte Imaging system was used to follow the cancer cells and quantify apoptosis by the presence of caspace-3/7 dye (see Materials and Methods). TAbs derived from both the TMab4 and 3E10 antibodies (T18 and T2_11) were selected for further investigation as both had demonstrated good killing. As can be seen in FIGS. 9A-9B both TAbs produced specific cell killing well above that produced by the effector cells alone or by effector cells cocultured with cell treated with the parental antibody. This shows that the immunogenic peptides are not only transferred into the target cells, but are processed and displayed on the cell surface which enables specific killing by effector cells.

The other TAbs are also tested using the IncuCyte Imaging assay. Specific killing above that induced by the effector cells alone and/or the parental antibody is observed with the TAbs of the invention.

The sequences of the various TAbs produced, and the parental antibodies are summarized in Table 12. It will be understood that when a heavy chain contains the immunogenic peptide the light chain of the parent is used to make the TAb and when the light chain contains the immunogenic peptide the heavy chain of the parent is used to make the TAb. Notably, the 3E10 antibody has two variants (called P2 and P3) with different light chains. Either can be used for generating TAbs.

TABLE 12
Antibody sequences

| | | SEQ | | | SEQ |
|---|---|---|---|---|---|
| Name | Chain | ID NO: | Name | Chain | ID NO: |
| TMab04- P1 | VH | 1021 | T1_30 | VH | 1045 |
| TMab04- P1 | VL | 1022 | T1_31 | VL | 1046 |
| 3E10-P2/P3 | VH | 1023 | T1_32 | VH | 1047 |
| 3E10-P2 | VL | 1024 | T1_33 | VH | 1048 |
| 3E10-P3 | VL | 1025 | T1_34 | VH | 1049 |
| 71F12-P4 | VH | 1026 | T1_35 | VH | 1050 |
| 71F12-P4 | VL | 1027 | T1_39 | VH | 1051 |
| T1 | VH | 1028 | T1_40 | VH | 1052 |
| T2 | VH | 1029 | T1_41 | VH | 1053 |
| T3 | VH | 1030 | T1_42 | VH | 1054 |
| T4 | VH | 1031 | T1_43 | VH | 1055 |
| T5 | VH | 1032 | T1_44 | VL | 1056 |
| T7 | VH | 1033 | T1_45 | VL | 1057 |
| T8 | VH | 1034 | T1_46 | VH | 1058 |
| T9 | VH | 1035 | T1_47 | VH | 1059 |
| T10 | VH | 1036 | T2_20 | VL | 1060 |
| T11 | VH | 1037 | T2_23 | VL | 1061 |
| T12 | VH | 1038 | T2_6 | VL | 1062 |
| T13 | VH | 1039 | T2_11 | VL | 1063 |
| T14 | VH | 1040 | T2_12 | VL | 1064 |
| T15 | VL | 1041 | T2_13 | VL | 1065 |
| T16 | VL | 1042 | T4_1 | VL | 1066 |
| T18 | VH | 1043 | T4_3 | VH | 1067 |
| T19 | VH | 1044 | | | |

Various known therapeutic antibodies were examined for inert CDRs. Crystal structure analysis for each CDR in contact with its antigen was performed and the number of amino acids contacting the antigen was counted. A distance of less than 5 angstroms between an amino acid of the CDR and an amino acid of the antigen was considered contact. More than 2 amino acids in contact with the antigen indicates a CDR involved in binding. Fewer than 2 indicates a definitely inert CDR. Exactly 2 indicates a CDR that is unlikely to be involved in binding or lowly involved. For purposes of evaluation all such CDRs are considered inert. Numerous other antibodies were found that contained inert CDRs which could be replaced with immunogenic peptides such as those provided in Tables 1-3. The MBR is used to optimize positioning. Alternatively, TAbs are designed by identifying peptides from Table 3 with sequence homology to known antibodies and specifically those with inert CDRs. Peptides with high homology are swapped into the antibody to produce TAbs. All produced Tabs are checked for cell penetrance and penetrance is retained. TAbs are also check for specific cell killing as described hereinabove and levels of killing above those produced by the parental antibody are observed.

### Example 8: Trojan Antibody Combined with a Targeting Antibody (Bispecific Trojan, Bi-TAb)

The TAbs described hereinabove can be considered killing modules that they have been shown to enter into target cells and induce specific killing by effector cells primed against the immunogenic peptide. However, in order to increase the specificity of these molecules, target them to cancer cells and reduce off-target effects a targeting module was added. Three EGFR antibodies were selected to use as the targeting module: Cetuximab, Panitumumab and Necitumumab. All three are known to specifically bind to EGFR and to target to EGFR overexpressing cancers. Antibodies targeting to other cancer specific/overexpressed molecules are also possible. As this is merely the targeting module, and is distinct from the killing module, any known cancer targeting antibody can be used. EGFR is used merely as a proof of concept.

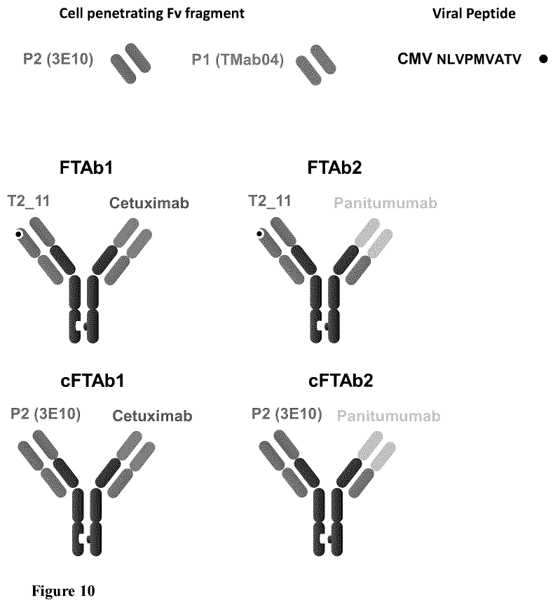

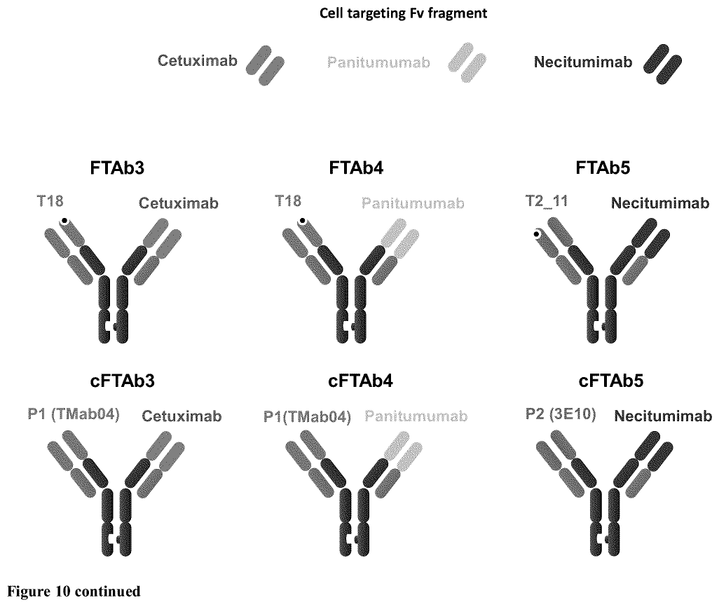

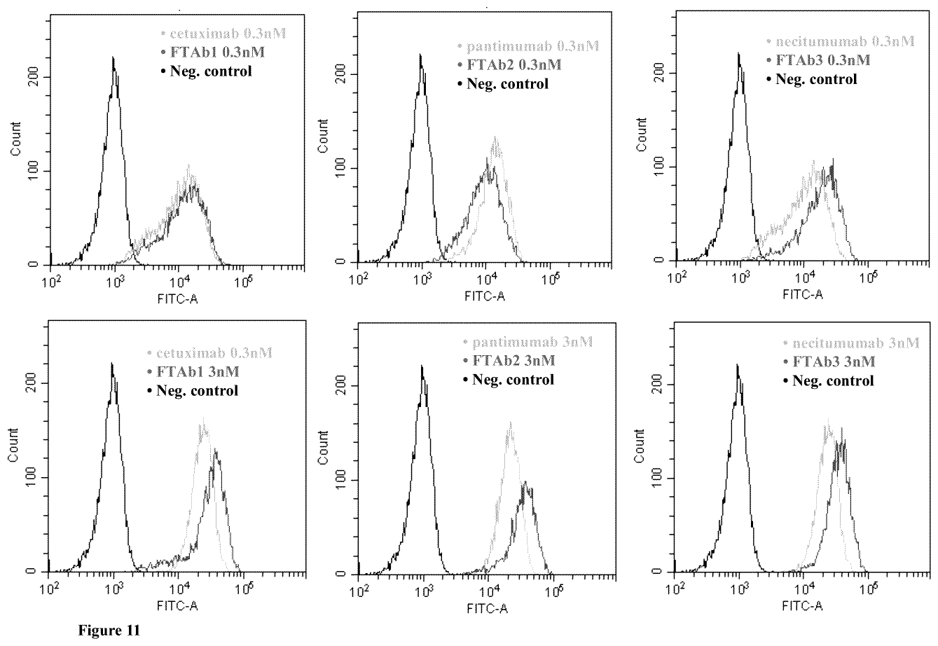

The T18 and T2_11 TAbs were used for the generation of bi-TAbs as a proof of principle. Control bi-functional antibodies were also generated using the TMab4 or 3E10 parental antibodies. The bispecific molecules were generated using the known knob-in-holes approach for restricting heavy chain homodimerization. The unique modifications are made in the constant region of the heavy chain of both the killing module and the targeting module (see Table 13, see the method provided in Shatz et al., “Knob-into-holes antibody production in mammalian cell lines reveals that asymmetric afucosylation is sufficient for full antibody-dependent cellular cytotoxicity”, 2013, mAbs 5:6, 872-881, herein incorporated by reference in its entirety). This promotes heterodimerization between the different heavy chains and discourages homodimerization. The produced bi-Tabs were found to have at least 90% purity for the desired dual-function molecule. Schematics of the final bi-TAbs and their controls are shown in FIG. 10 and the full sequences are provided in Table 13.

TABLE 13
Sequences of targeting arms and bifunctional antibodies.

| | | SEQ ID |
|---|---|---|
| Name | Chain | NO: |
| cFTAb1_arm1_P2 | LC | 1077 |
| cFTAb1_arm1_P2 | HC | 1078 |
| cFTAb1_arm2_Cetuximab | LC | 1079 |
| cFTAb1_arm2_Cetuximab | HC | 1080 |
| cFTAb2_arm1_P2 | LC | 1077 |
| cFTAb2_arm1_P2 | HC | 1078 |
| cFTAb2_arm2_Panitumumab | LC | 1081 |
| cFTAb2_arm2_Panitumumab | HC | 1082 |
| cFTAb3_arm1_P1 | LC | 1083 |
| cFTAb3_arm1_P1 | HC | 1084 |
| cFTAb3_arm2_Cetuximab | LC | 1079 |
| cFTAb3_arm2_Cetuximab | HC | 1080 |
| cFTAb4_arm1_P1 | LC | 1083 |
| cFTAb4_arm1_P1 | HC | 1084 |
| cFTAb4_arm2_Panitumumab | LC | 1081 |
| cFTAb4_arm2_Panitumumab | HC | 1082 |
| cFTAb5_arm1_P2 | LC | 1077 |
| cFTAb5_arm1_P2 | HC | 1078 |
| cFTAb5_arm2_Necitumumab | LC | 1085 |
| cFTAb5_arm2_Necitumumab | HC | 1086 |
| FTAb1_arm1_T2_11 | LC | 1087 |
| FTAb1_arm1_T2_11 | HC | 1088 |
| FTAb1_arm1_Cetuximab | LC | 1079 |
| FTAb1_arm1_Cetuximab | HC | 1080 |
| FTAb2_arm1_T2_11 | LC | 1087 |
| FTAb2_arm1_T2_11 | HC | 1088 |
| FTAb2_arm2_Panitumumab | LC | 1081 |
| FTAb2_arm2_Panitumumab | HC | 1082 |
| FTAb3_arm1_T18 | LC | 1089 |
| FTAb3_arm1_T18 | HC | 1090 |
| FTAb3_arm2_Cetuximab | LC | 1079 |
| FTAb3_arm2_Cetuximab | HC | 1080 |
| FTAb4_arm1_T18 | LC | 1089 |
| FTAb4_arm1_T18 | HC | 1090 |
| FTAb4_arm2_Panitumumab | LC | 1081 |
| FTAb4_arm2_Panitumumab | HC | 1082 |
| FTAb5_arm1_T2_11 | LC | 1087 |
| FTAb5_arm1_T2_11 | HC | 1088 |
| FTAb5_arm2_Necitumumab | LC | 1085 |
| FTAb5_arm2_Necitumumab | HC | 1086 |

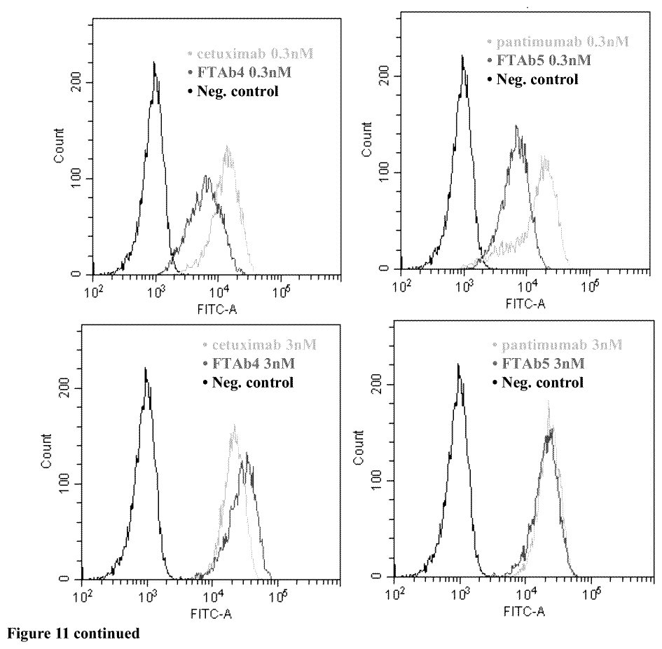

The bi-TAbs were tested for their ability to bind surface EGFR on breast cancer cells that express high levels of EGFR. It is predicted that the additional killing module should not impact surface binding of the targeting module and indeed that is what is observed (FIG. 11). The bi-TAbs at either a concentration of 0.3 nM or 3 nM successfully bound surface EGFR and at a level comparable to the control original anti-EGFR antibody. Thus, the bi-TAbs can effectively target to cancer cells based on the targeting module selected.

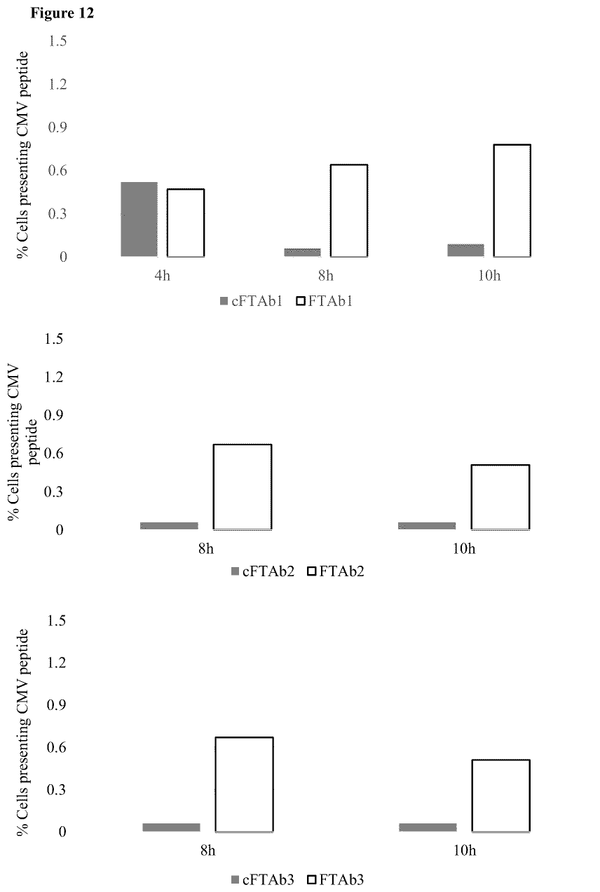

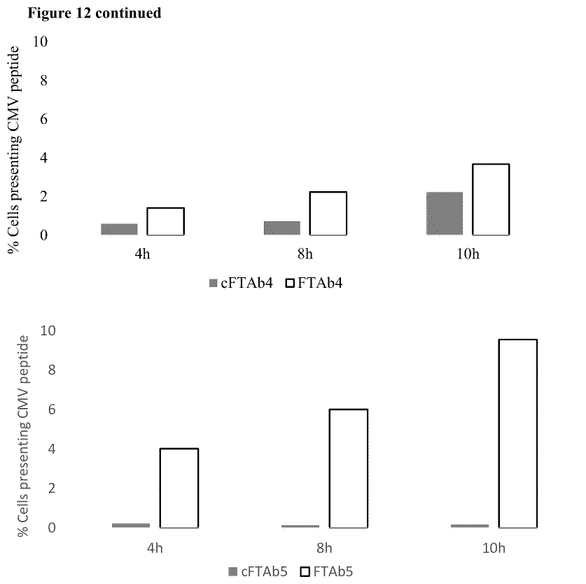

Next, the bi-TAbs ability to bring the immunogenic peptide on to the surface of the cancer cells in complex with an HLA molecule was tested. HLA-peptide display was measured as described hereinabove (Materials and Methods). A time course experiment was performed to monitor surface display over time. All five of the bi-TAbs successfully brought the peptide to the cell surface by 10 hours of incubation (FIG. 12). All bi-Tabs were superior to their control counterparts.

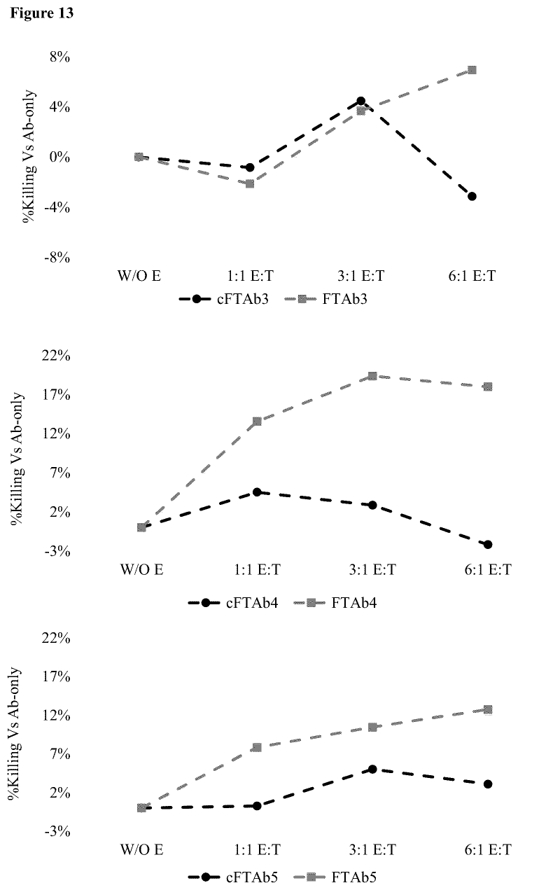

Finally, cancer cell killing was examined at various effector cell concentration. Bi-TAbs 3-5 each with a different targeting module were tested at effector:target cell (E:T) concentrations of 1:1, 3:1 and 6:1. At a ratio of 6:1 all three antibodies induced specific killing above what is observed with the control antibodies (FIG. 13). Bi-TAbs 4 and 5 (one which contains the P1 derived TAb and one which contains the P2 derived TAb) also showed increased killing at ratios of 3:1 and even 1:1. A culture without effector cells was used as a control. These results demonstrate the high level of effectiveness of the bi-TAb as it combines cancer cell targeting with high levels of specific killing.

The three anti-EGFR antibodies used themselves contain inert CDRs. A summary of these inert CDRs is provided in Table 14. CDRH1, CDRL1 and CDRL2 of Cetuximab, CDRL1 and CDRL2 of Panitumumab and CDRL1 and CDRL2 of Necitumumab were all found to be inert. These CDRs are also replaced with immunogenic peptides. The targeting of the replaced antibodies is confirmed and the ability to bind to EGFR and target to EGFR overexpressing cancer cells is retained. The combination of the modified targeting module to the killing module increases the number of immunogenic peptides delivered. The peptide used in the killing module and the peptide used in the targeting module can be the same or different.

TABLE 14
Inert CDRs in anti-EGFR antibodies.

| | CDRH1 | CDRH2 | CDRH3 | CDRL1 | CDRL2 | CDRL3 | Inert |
|---|---|---|---|---|---|---|---|
| Antibody | score | score | score | score | score | score | CDRs |
| Cetuximab | 1 | >2 | >2 | 2 | 1 | >2 | H1, L1, |
| | | | | | | | L2 |
| Panitumumab | >2 | >2 | >2 | 2 | 1 | >2 | L1, L2 |
| Necitumumab | >2 | >2 | >2 | 2 | 0 | >2 | L1, L2 |

Bi-TAbs are generated with the other killing modules and various targeting modules. The bi-Tabs are evaluated as above to cancer cell targeting, HLA-peptide surface display and for specific killing. Bi-Tabs are superior to the control bi-functional molecule and are able to target to cancers, induce peptide display and ultimately enhance killing of the target cells by effector cells specific to the immunogenic peptide.

In vivo validation is also performed. The engineered bi-Tabs are injected into immune competent mice expressing a tumor targetable by the targeting module. Control bi-functional molecules lacking the immunogenic peptide and/or binding a non-cancer related target are also administered. Animal survival over time is monitored as is tumor size. The Trojan antibodies are found to shrink tumors and/or extend survival time in a statistically significant manner, indicating that they activate the immune system against the cancer. Mice may be vaccinated with the immunogenic peptide beforehand.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A dual-function antigen binding molecule comprising:
a. a first antibody or antigen binding fragment thereof comprising at least one immunogenic peptide inserted into a CDR of said antibody or antigen binding fragment thereof, and where said insertion comprises removal of CDR sequence; and
b. a second antibody capable of binding epidermal growth factor receptor (EGFR), wherein said antibody is selected from cetuximab, panitumumab and necitumumab or antibody comprising at least 85% sequence identity thereto.

2. The dual function antigen binding molecule of claim 1, wherein said first antibody or antigen binding fragment thereof and said second antibody comprise at least one modification that promotes heterodimerization and inhibit homodimerization, and wherein one of said first and second antibody comprises a heavy chain constant region comprising SEQ ID NO: 1074 and the other antibody comprises a heavy chain constant region comprising SEQ ID NO: 1075.

3. (canceled)

4. The dual function antigen binding molecule of claim 1, wherein said first antibody or antigen binding fragment thereof binds to a target cell, and wherein said target cell is a cancer cell and a cancer cell antigen is selected from HER2, EGFR, EpCAM, PSMA, BCMA, CD123, CD33, CD38, CTLA, LAG-3, ICOS, 4-1BB and PD-L1, a dendritic cell and a dendritic cell antigen is selected from CD40, CD205, CD206, CLEC9A, CLEC12A, CD209, and CD207 or both.

5. The dual function antigen binding molecule of claim 1, wherein said immunogenic peptide is a cancer specific peptide, is a viral peptide, is a peptide recognized by CD4 T cells, CD8 T cells or a combination thereof.

6. The dual function antigen binding molecule of claim 5, wherein said peptide is a cancer specific peptide and is selected from a peptide sequence provided in Table 1.

7. (canceled)

8. The dual function antigen binding molecule of claim 5, wherein said peptide is a viral peptide is-derived from Cytomegalovirus (CMV), Epstein-Barr virus (EBV), Severe acute respiratory syndrome coronavirus 2 (SARS-CoV2), Adenovirus, Human papilloma virus (HPV) or Influenza virus (FLU) and is selected from a peptide sequence provided in Table 2 or Table 3.

9. (canceled)

10. The dual function antigen binding molecule of claim 1, further comprising a cell penetration sequence that targets said first antibody or antigen binding fragment thereof to a cytoplasm of a cell bound by said first antibody or antigen binding fragment thereof and wherein said cell penetration sequence is an endosomal escape domain (EED).

11. (canceled)

12. (canceled)

13. The dual function antigen binding molecule of claim 1, wherein said CDR is an inert CDR having little or no contribution to binding to a target antigen.

14. The dual function antigen binding molecule of claim 13, wherein an inert CDR comprises two or fewer amino acids that contact said target antigen and wherein contact comprises a distance of not more than 5 angstroms between an amino acid of a CDR and an amino acid of said target antigen.

15. (canceled)

16. The dual function antigen binding molecule of claim 1, wherein said insertion and removal produces no change or minimal change in the overall conformation of said first antibody or antigen binding fragment thereof such that said first antibody or antigen binding fragment thereof binds its target antigen at an equivalent affinity to said first antibody or antigen binding fragment devoid of said immunogenic peptide.

17. The dual function antigen binding molecule of claim 1, wherein at least one inert CDR of an antigen binding region is replaced with a cell penetration sequence.

18. (canceled)

19. (canceled)

20. (canceled)

21. (canceled)

22. The dual function antigen binding molecule of claim 1, wherein within said first antibody or antigen binding fragment thereof said antigen binding region, said immunogenic peptide and said cell penetrating sequence are each separated by a linker, wherein said dual function antigen binding molecule is devoid of a chemical linker, or both.

23. (canceled)

24. The dual function antigen binding molecule of claim 1, wherein said first antibody before said immunogenic peptide is inserted is selected from:
a. antibody TMab4 comprising a heavy chain variable region of SEQ ID NO: 1021 and a light chain variable region of SEQ ID NO: 1022;
b. antibody 3E10 comprising a heavy chain variable region of SEQ ID NO: 1023 and a light chain variable region of SEQ ID NO: 1024; and
c. antibody 71F12 comprising a heavy chain variable region of SEQ ID NO: 1026 and a light chain variable region of SEQ ID NO: 1027; and
d. said immunogenic peptide is inserted into CDRH1, CDRH2, CDRH3 or CDRL3 of said TMab4, CDRL1 or CDRL2 of said 3E10 or CDRL1 of said 71F12.

25. (canceled)

26. The dual function antigen binding molecule of claim 24, wherein said first antibody comprises at least one of:
a. a light chain variable region of SEQ ID NO: 1022 and a heavy chain variable region selected from SEQ ID NO: 1028-1040, 1043-1045, 1047-1055, and 1058-1059;
b. a heavy chain variable region of SEQ ID NO: 1021 and a light chain variable region selected from SEQ ID NO: 1041-1042, 1046, and 1056-1057;
c. a heavy chain variable region of SEQ ID NO: 1023 and a light chain variable region selected from SEQ ID NO: 1060-1065;
d. a heavy chain variable region of SEQ ID NO: 1026 and a light chain variable region of SEQ ID NO: 1066; and
e. a light chain variable region of SEQ ID NO: 1027 and a heavy chain variable region of SEQ ID NO: 1067;
said dual function antigen binding molecule comprises two heavy chains and two light chains and wherein:
a. said two heavy chains are SEQ ID NO: 1088 and 1080 and said two light chains are SEQ ID NO: 1087 and 1079;
b. said two heavy chains are SEQ ID NO: 1088 and 1082 and said two light chains are SEQ ID NO: 1087 and 1081;
c. said two heavy chains are SEQ ID NO: 1090 and 1080 and said two light chains are SEQ ID NO: 1089 and 1079;
d. said two heavy chains are SEQ ID NO: 1090 and 1082 and said two light chains are SEQ ID NO: 1089 and 1081; or
e. said two heavy chains are SEQ ID NO: 1088 and 1086 and said two light chains are SEQ ID NO: 1087 and 1085; or
both.

27. (canceled)

28. An antibody or antigen binding fragment thereof comprising at least one immunogenic peptide inserted into a variable region of said antibody or antigen binding fragment thereof, and where said insertion comprises removal of antibody or antigen binding fragment sequence.

29. A pharmaceutical composition comprising a dual-function antigen binding molecule of claim 1 and a pharmaceutically acceptable carrier excipient or adjuvant.

30. A nucleic acid molecule comprising at least one open reading frame, wherein said open reading frame encodes a dual-function antigen binding molecule of claim 1, optionally wherein said nucleic acid molecule is an expression vector and comprises at least one regulatory element operatively linked to said open reading frame.

31. (canceled)

32. A method of treating EGFR positive cancer in a subject in need thereof, the method comprising administering to said subject a pharmaceutical composition of claim 29, thereby treating cancer in a subject.

33. (canceled)

34. A method of engineering an antibody or antigen binding fragment thereof, the method comprising:
a. selecting an antibody or antigen binding fragment thereof of interest;
b. receiving structural analysis of said selected antibody or antigen binding domain bound to its target;
c. determining at least one CDR of said selected antibody or antigen binding domain that is not required for binding to said target based on said structural analysis;
d. replacing said determined at least one CDR or a portion thereof with an immunogenic peptide; or
a. selecting an antibody or antigen binding fragment thereof of interest;
b. receiving a database of immunogenic peptides;
c. performing pairwise alignment of peptides of a variable region of said selected antibody or antigen binding fragment thereof of interest with immunogenic peptides of said database;
d. determining a peptide from said selected antibody or antigen binding fragment thereof and an immunogenic peptide with an alignment score above a predetermined threshold; and
e. replacing said determined peptide from said selected antibody or antigen binding fragment thereof with said determined immunogenic peptide;
thereby engineering an antibody or antigen binding fragment thereof.

35. (canceled)

36. The method of claim 34, where at least one of.
a. said method further comprises optimizing said replacing to produce as little perturbation in the structure of said selected antibody or antigen binding fragment thereof of interest as possible,
b. said engineered antibody or antigen binding fragment thereof is an immunogenic peptide delivery antibody;
c. step (a) comprises selecting an antibody or antigen binding fragment thereof that binds to a surface of a target cell;
d. step (a) comprises selecting an antibody or antigen binding fragment thereof that binds to a surface of a target cell and upon binding to a surface is internalized and delivered to a cytosol of said target cell;
e. said method further comprises confirming at least one of: delivery of said immunogenic peptide to a cytosol of said target cell, delivery of said immunogenic peptide in complex with an HLA molecule to a surface of said target cell and specific killing of said target cell by an effector cell specific to said immunogenic peptide; and
f. wherein said method further comprises selecting a targeting antibody that binds to a protein on a surface of a target cell and producing a dual-function antigen binding molecule by combining said engineered antibody and said targeting antibody, optionally wherein combining comprises engineering a heavy chain constant region of said targeting antibody and a heavy chain constant region of said engineered antibody to promote heterodimerization and inhibit homodimerization.

37. (canceled)

38. (canceled)

39. (canceled)

40. (canceled)

41. (canceled)

42. (canceled)
